(19) United States
(12) Patent Application Publication
(10) Pub. No.: US 2025/0361490 A1
(43) Pub. Date: Nov. 27, 2025

(54) COMPOSITION AND METHOD FOR CRYOPRESERVATION OF MITOCHONDRIA

(71) Applicant: cellvie AG, Zürich (CH)

(72) Inventors: Dedy SEPTIADI, Zürich (CH); Oleksandr LYTOVCHENKO, MöIndal (SE); Nina DUMAUTHIOZ, Bruxelles (BE)

(21) Appl. No.: 18/873,304

(22) PCT Filed: Jun. 12, 2023
(86) PCT No.: PCT/EP2023/065697

§ 371 Date: Dec. 9, 2024

(30) Foreign Application Priority Data
Jun. 10, 2022 (EP) 22178518.1

(51) Int. Cl. C12N 5/00; A61K 35/12

(52) U.S. Cl. CPC C12N 5/525; A61K 35/12; C12N 5/526; C12N 5/562

(57) ABSTRACT

The disclosure relates to composition and method for cryopreservation of mitochondria, cryopreserved composition comprising mitochondria and their therapeutic use.

### FIELD

The present application relates to compositions and methods for the cryopreservation of compositions comprising isolated viable mitochondria. The present application further relates to cryopreserving compositions used in the method described herein and to cryopreserved compositions comprising isolated viable mitochondria obtained with the method described herein. These cryopreserved compositions comprising mitochondria exhibit an advantageous and valuable long shelf-life time and are ready-to-use for numerous purposes, which may include, but are not limited to, research, diagnostic and therapeutic applications.

### BACKGROUND

Mitochondria are double membrane-bound organelles found in the cytoplasm of nucleated eukaryotic cells. They are found in almost every cell of the human body except red blood cells and vary in number and location depending on the cell type. Mitochondria perform numerous essential tasks in the eukaryotic cell such as pyruvate oxidation, the Krebs cycle (i.e., the citric acid cycle) and metabolism of amino acids, fatty acids, and steroids. Mitochondria are also involved in processes such as heat production, cell differentiation, cell information transmission, storage of calcium ions, calcium signaling, and programmed cell death (apoptosis), and have the ability to regulate the cell growth cycle. Mitochondria are the cell's primary site of energy metabolism and generate adenosine triphosphate (ATP) for different cell functions. Since adenosine triphosphate is the energy source for cellular activities, mitochondria are also known as “cellular energy factories”. Mitochondria generate ATP by means of the electron-transport chain and the oxidative phosphorylation system (the “respiration chain”). Typically, more than 90% of a cell's requirement for ATP is supplied by the cell's own mitochondria. (Cell Biology of the Mitochondrion Genetics (November 2017), 207(3):843-871, Van der Bliek A. M., Sedensky M. M., Morgan P. G.; Erratum in: Genetics (April 2018), 208(4):1673; Mitochondria: in sickness and in health Cell (16 Mar. 2012), 148(6):1145-59, Nunnari J, Suomalainen A.).

Mitochondria are composed of two concentric membranes, which have specialized functions. The inner mitochondrial membrane contains proteins needed for respiratory chain function and for ATP synthesis. The outer mitochondrial membrane, which contains large numbers of integral membrane proteins encloses the entire organelle. The structure of mitochondria has striking similarities to some modem prokaryotes. In fact, mitochondria are thought to have originated from an ancient symbiosis when a nucleated cell engulfed an aerobic prokaryote. In the symbiosis relationship, the host cell came to rely on the engulfed prokaryote for energy production, and the prokaryote cell began to rely on the protective environment provided by the host cell (The Origin of Mitochondria. Nature Education (2010), 3(9):58, Martin W. & Mentel M.)

As ubiquitous, semi-autonomous cellular organelles, mitochondria are separated from the cytoplasm by the outer and inner mitochondrial membrane. The outer membrane is porous and freely traversed by ions and small, uncharged molecules through pore-forming membrane proteins (porins), such as the voltage-dependent anion channel (VDAC). Any larger molecules, especially proteins, have to be imported by special translocases. Because of its porosity, there is no membrane potential across the outer membrane. By contrast, the inner membrane is a tight diffusion barrier to all ions and molecules. These can only get across with the aid of specific membrane transport proteins, each of which is selective for a particular ion or molecule. As a result of its ion selectivity, the electrochemical membrane potential builds up across the inner mitochondrial membrane. The inner membrane is where oxidative phosphorylation takes place in a suite of membrane protein complexes that create the electrochemical gradient across the inner membrane or use it for ATP synthesis. The outer membrane separates mitochondria from the cytoplasm. It surrounds the inner membrane, which separates the inter-membrane space from the protein-dense central matrix. The inner membrane is differentiated into the inner boundary membrane and the cristae. The two regions are continuous at the crista junctions. The cristae extend more or less deeply into the matrix and are the main sites of mitochondrial energy conversion. Due to mitochondria's primary function in cell metabolism, damage and dysfunction in mitochondria can cause a range of human diseases. Mitochondria are subcellular organelles, which are indispensable for cells' life. Disruption of normal mitochondrial function is detrimental to cell viability. Mitochondrial disorders may be caused by mutations (acquired or inherited), in mitochondrial DNA (mtDNA), or in nuclear genes that code for mitochondrial components. The inherited mitochondrial disorders may include, but are not limited to, mitochondrial encephalopathy, mitochondrial myopathy, lactic acidosis and stroke-like episodes (MELAS) syndrome, myoclonic epilepsy with ragged red fibers (MERRF), neuropathy, ataxia and retinitis pigmentosa (NARP) syndrome, myoneurogenic gastrointestinal encephalopathy (MNGIE), Leber's hereditary optic neuropathy (LHON, and mitochondrial DNA depletion syndrome (Mol Syndromol (2016), 7:122-137, Nyazov D. M. et al.). Damage to mitochondria may also be caused by injury, toxicity, chemotherapy, infections, and age-related changes. Reduction of blood flow in tissues and organs may produce mitochondrial damage. Particularly, ischemia/reperfusion injury can cause mitochondrial damage, which will have a negative impact on oxygen consumption and energy synthesis (Annual Review of Pharmacology and Toxicology, Vol. 57, (2017), Lesnefsky, pp 535-565; A Cell Metabolism Review, (2015), Unifying Mechanism for Mitochondrial Superoxide Production during Ischemia-Reperfusion Injury, Chouchan E. T.). In general, mitochondrial or mitochondria-related diseases/disorders are chronic (long-term) diseases/disorders. The symptoms of mitochondrial disease can vary. It depends on how many mitochondria are defective, and where they are in the body. Sometimes only one organ, tissue, or cell type is affected. But often the problem affects many of them. Muscle and nerve cells have especially high energy needs, so muscular and neurological problems are common. The diseases range from mild to severe. Some types can be fatal. Mitochondria-related diseases, such as acquired mitochondria-related diseases or dysfunction, may include, but are not limited to, central nervous system (CNS) diseases such as Alzheimer's disease, Parkinson's disease, Alpers syndrome, Leigh syndrome, and myoclonic epilepsy with ragged red fibers, as well as dysfunction or diseases following stroke and traumatic brain or spinal cord injury (Mitochondrion (July 2017), 35:70-79, Gollihue and Rabchevsky). Drugs can induce mitochondrial dysfunction by different mechanisms including inhibition of fatty acid oxidation, impairment of oxidative phosphorylation and respiratory chain activity as well as alteration of the integrity of the mitochondrial membranes. Some drugs also impair mitochondrial function via the production of reactive oxygen species and the generation of reactive metabolites, which can covalently bind to key mitochondrial proteins. Drug-induced mitochondrial dysfunction plays an important role in the pathogenesis of adverse effects such as liver injury, myopathy, and cardiotoxicity. Examples of drugs, which can induce mitochondrial dysfunction are, for instance, acetaminophen, amiodarone, doxorubicin, nucleoside reverse transcriptase inhibitors (e.g., stavudine, zidovudine, didanosine), statins (e.g., atorvastatin, cerivastatin, simvastatin) and valproic acid (Mitochondrial Biology and Experimental Therapeutics, (March 2018), pp 269-295, Massart).

The isolated viable mitochondria of the invention are particularly useful for the treatment of mitochondrial or mitochondria-related diseases and disorders, genetic or acquired mitochondrial dysfunctions, cancer, infectious diseases, inflammatory diseases, autoimmune diseases, ischemia, ischemia-related dysfunctions, ischemia reperfusion injuries, or blood clot blockages as the other diseases and dysfunctions described above. Particularly important are emerging treatments such as therapeutic mitochondrial transplantation (J Transl Med, (17 May 2021), 19(1):214, Hayashida K; EMBO Rep. 2020 Sep. 3; 21(9): e50964, published online 2020 Aug. 2, R. N. Lightowlers et al.; J. et al.; Int J Mol Sci. 2021 May; 22(9): 4793, published online 2021 Apr. 30, A. Park et al.).).

Cryopreservation of subcellular organisms/organelles is a process that allows the preservation of subcellular organisms, such as mitochondria, after isolating them from the cell by cooling their samples to very low temperatures. Mitochondria, which have great potential for use in basic research as well as for many medical applications, cannot be stored with simple cooling or freezing for a long time, for the reason that ice crystal formation, e.g., intracellular ice formation and/or dendritic ice crystal formation, osmotic shock, e.g., the solution effect injury described by Peter Mazur (1977), and membrane damage during freezing and thawing, will cause mitochondria damage or death. Many attempts have been made to cryopreserve mitochondria with the use of cryoprotective agents and temperature control equipment (Biochim. Biophys. Acta (1961) 50: 233-242, D. Greiff; M. Myers, C. A. Privitera; Nature (1961) 190: 1202-1204, Greiff; D. and M. Myers). However, cryopreservation known in the art gives rise to mitochondria damage during freezing, freezing-thawing, and storage at low temperatures. This degradation has a deleterious effect on the activity and viability of mitochondria and hence their potential use for research, diagnostic, or therapeutic purposes. The inability to store mitochondria over longer periods of time severely limits the possible uses of mitochondria, particularly in a clinical environment, where there is often not enough time to freshly prepare mitochondria. Additionally, mitochondria isolation is time consuming and a process that is difficult to scale up within a single laboratory or hospital, leading to high costs of preparation. This is particularly problematic for therapeutic uses, where the preparation of the product for use on the patient would have to adhere to Good Manufacturing Practice. Therefore, there is still a strong need for compositions and methods for cryopreservation of mitochondria, which are capable of maintaining the structural and/or functional integrity of mitochondria, preferably both the structural and functional integrity of mitochondria.

It would be extremely advantageous to have at one's disposal compositions comprising isolated mitochondria, which can be stored for long periods of time, and which are ready-to-use on an as-needed basis, for different purpose, such as, basic research and/or medical applications. Having at one's disposal off-the-shelf composition comprising healthy mitochondria would render the administration of viable mitochondria to a patient (e.g., by transplantation of mitochondria, such as, by replacing defective mitochondria within a cell or tissue or by enriching a cell and tissue affected by the disease or dysfunction with healthy mitochondria) expedient and effective. Thereby, mitochondrial therapies would become routine procedures, which could enable the prevention, improvement or elimination of symptoms linked to mitochondria which are either malfunctioning or insufficient in number. Therapeutic Mitochondria Transplantation (TMT) holds the potential of sustainably affecting mitochondria function, reinvigorating, or amplifying the cellular energy metabolism. For instance, McCully et al. describes that mitochondrial transplantation can be effective in a number of cell types and diseases. These include cardiac and skeletal muscle, pulmonary and hepatic tissues and cells, and neuronal tissue. McCully foresees that mitochondrial transplantation will be a valued treatment in the armamentarium of all clinicians and surgeons for the treatment of varied ischemic disorders, mitochondrial diseases, and related disorders (Clin Trans Med (2016) 5:16, McCully et al.).

For this type of therapeutic uses at the bed-side of the patient in need of treatment, ready-to-use compositions comprising mitochondria would streamline the entire clinical procedure by rendering it, for example, safer, quicker, more consistent, and easier to perform.

Since mitochondria have a double-membrane structure and are more fragile than cells, although a variety of cryopreservatives or cryopreservatives mixture for cells' preservation are known, these cryopreservatives are not suitable for freezing mitochondria. To this end, the industry is actively attempting to develop mitochondria-specific cryopreservatives. However, during mitochondrial cryopreservation, mitochondria are still very often swollen, damaged or even ruptured, preventing mitochondria from functioning properly, for instance after thawing. Therefore, the preservation of mitochondria from being damaged during cryopreservation is still a not fully solved issue.

Mitochondria play an important role in the regulation of the apoptosis of the cells. Permeabilization of the mitochondrial outer membrane is a critical step in the apoptotic process. The outer membrane of isolated mitochondria tends to deteriorate over time and rupture upon freeze-thaw. By analyzing the cytochrome c retention over time at different temperatures, Yamaguchi et al. showed that mitochondria that have been frozen in a buffer comprising trehalose preserve mitochondrial outer membrane integrity (Yamaguchi et al., Cell Death and Differentiation (2007), (14):616-624). Yamaguchi et al. also attempted to demonstrate that trehalose-frozen mitochondria are biologically similar to freshly prepared mitochondria. Indeed, Yamaguchi et al. claimed that mitochondria that have been frozen in a buffer comprising trehalose allegedly retain a number of biological and bioenergetics functions, such as, for instance, the capacity of synthetizing ATP, responding to increasing concentration of calcium by activating the permeability transition pore (PTP), retaining transmembrane inner potential, and importing and processing protein. Yet, the data presented in Yamaguchi et al. did not sufficiently and effectively prove that those mitochondrial functions are maintained, and the results are therefore inconclusive. For instance, the significantly decreased rates of both phosphorylating and maximally uncoupled respiration are indicative of a compromised bioenergetic function. Indeed, Yamaguchi et al., acknowledged that at closer examination almost most of the mitochondrial functions appeared to be impaired.

US 2019/0134088 A1 describes partially purified functional mitochondria, which are derived from a cell or a tissue and which have undergone a freeze-thaw cycle. The freezing buffer comprises as cryoprotectant a saccharide, an oligosaccharide, or a polysaccharide. In US 2019/0134088 A1 it is disclosed that a sufficient saccharide concentration which acts to preserve mitochondrial function is a concentration of between 100 mM-400 mM. Therein, it is also disclosed that the partially purified mitochondria, which have undergone a freeze-thaw cycle, could be stored as frozen mitochondria for at least 1, 2, 3 weeks or even 1 month prior to thawing. In particular, the saccharide used in US 2019/013088 A1 was sucrose and most importantly the saccharide was always required to be other than trehalose or mannitol. Yet, throughout the entire disclosure of US 2019/0134088 A1, not enough evidence was provided in support of the fact that partially purified mitochondria remained intact and viable after the freeze-thaw cycle or after storage. Likewise, the findings that sucrose alone could be deemed to be a sufficiently good cryoprotectant for mitochondria seems to be in stark conflict with the conclusions of Yamaguchi et al.

WO 2021/168764 A1 (Taiwan Mitochondrion Applied Tech Co Ltd) discloses a mitochondrial cryopreservation agent and a cryopreservation method. It describes a first cryopreservative composition consisting of 300 mM of trehalose, 10 mM of 2-[4-(2-hydroxyethyl)-piperazin-1-yl]-ethanesulfonic acid (i.e., 10 mM of HEPES) and 0.1 wt. % of human serum albumin (HSA) for the cryopreservation of mitochondria. This cryopreservative composition does not contain KCl, NaCl, EDTA, EGTA, and ethylene glycol. Comparative cryopreservative composition 1 consists of 300 mM of trehalose, 10 mM of HEPES, 0.1 wt. % of HSA, 10 mM of KCl, 1 mM of EDTA and 1 mM EGTA. Comparative cryopreservative composition 2 consists of 300 mM of trehalose and 0.1 wt. % of HSA. The experimental data provided in WO 2021/168764 A1 attempted to demonstrate that the integrity of the mitochondrial outer membrane (MOM) of cryopreserved mitochondria is maintained, when the first composition is used. Yet, the data presented in WO 2021/168764 A1 do not in any way prove that those frozen mitochondria remain viable throughout the freeze-thaw cycle.

The technical problem is solved by the embodiments/items provided herein and as characterized in the claims. Accordingly, the invention relates to, inter alia, the following items:

1. A composition comprising:
(i) an aqueous buffer having a pH of 5.5 to 8.5
(ii) trehalose at a concentration of at least 150 mM;
(iii) a first cryoprotecting agent(s) selected from one or more (a) amino acid(s) at a total concentration of at least 150 mM; and optionally
(iv) a second cryoprotecting agent(s) selected from one or more (b) sugar(s), (c) polymer(s), or a combination thereof, wherein
   (b) the sugar(s) has a total concentration of at least 150 mM; and
   (c) the polymer(s) has a total concentration of 2.5% (w/v) to 30% (w/v).

2. A composition comprising:
(i) an aqueous buffer having a pH of 5.5 to 8.5
(ii) trehalose at a concentration of at least 150 mM;
(iii) a first cryoprotecting agent(s) selected from one or more (b) sugar(s) at a total concentration of at least 160 mM; and optionally
(iv) a second cryoprotecting agent(s) selected from (a) amino acid(s), (c) polymer(s), or a combination thereof, wherein
   (a) the amino acid has a total concentration of at least 150 mM; and
   (c) the polymer has a total concentration of 2.5% (w/v) to 30% (w/v).

3. A composition comprising:
(i) an aqueous buffer having a pH of 5.5 to 8.5
(ii) trehalose at a concentration of at least 150 mM; and
(iii) a cryoprotecting agent(s) selected from one or more (c) polymer(s), wherein the total concentration of the polymer(s) is from 16% (w/v) to 30% (w/v).

4. The composition of any one of items 1 to 3, wherein the pH of (i) the aqueous buffer is from 6.0 to 8.5, such as 6.5 to 8.2.

5. The composition of item 4, wherein the pH of (i) the aqueous buffer is from 6.8 to 8.0, such as 7.2.

6. The composition of any one of items 1 to 5, wherein (i) the aqueous buffer comprises a buffering agent, preferably wherein the buffering agent is selected from 2-[4-(2-hydroxyethyl)-piperazin-1-yl]-ethane-sulfonic acid (HEPES), piperazine-N,N′-bis(2-ethane-sulfonic acid) (PIPES), 4-morpholineethanesulfonic acid (MES), bis-(2-hydroxyethyl)amino-tris-(hydroxymethyl)-methane (Bis-Tris), 2-(N-cyclohexylamino)-ethane sulfonic acid (CHES), N,N-Bis-(2-hydroxyethyl)-glycine (Bicine), potassium phosphate, sodium cacodylate, tris-(hydroxymethyl)aminomethane hydrochloride) (Tris), 4-morpholinepropanesulfonic acid (MOPS), 1,3-bis-[tris-(hydroxymethyl)-methylamino]-propane (Bis-Tris propane), sodium acetate, or a combination thereof.

7. The composition of any one of items 1 to 6, wherein the buffering agent in the aqueous buffer has a concentration of 0.5 mM to 50 mM.

8. The composition of any one of items 1 to 7, wherein the buffering agent in the aqueous buffer has a concentration of 2 mM to 35 mM, such as 5 mM to 30 mM.

9. The composition of any one of items 1 to 8, wherein the buffering agent in the aqueous buffer has a concentration of 10 mM to 25 mM, such as 15 mM.

10. The composition of any one of items 1 to 9, wherein (i) the aqueous buffer is at pH 7.2 and comprises 10 mM 2-[4-(2-hydroxyethyl)-piperazin-1-yl]-ethanesulfonic acid (HEPES).

11. The composition of any one of items 1 to 10, further comprising a calcium chelator, wherein the calcium chelator is selected from the group consisting of ethylene glycol-bis(3-aminoethyl ether)-N,N,N′,N′-tetraacetic acid (EGTA), 2,2′,2″,2′″-(Ethane-1,2-diyldinitrilo)-tetraacetic acid (EDTA), 1,2-bis(o-aminophenoxy)ethane-N,N,N′,N′-tetraacetic acid (BAPTA), 1,2-bis(2-aminophenoxy)ethane-N,N,N′,N′-tetraacetic acid tetrakis-(acetoxymethyl ester) (BAPTA-AM) or a combination thereof.

12. The composition of item 11, wherein the calcium chelator has a concentration of 0.1 mM to 10 mM, such as 0.2 mM to 5 mM.

13. The composition of item 12, wherein the calcium chelator has a concentration of 0.5 to 2 mM, such as 1 mM.

14. The composition of any one of items 11 to 13, wherein the calcium chelator is ethylene glycol-bis(3-aminoethyl ether)-N,N,N′,N′-tetraacetic acid (EGTA) or 2,2′,2′,2′″-(Ethane-1,2-diyldinitrilo)-tetraacetic acid (EDTA).

15. The composition of any one of items 1 to 14, further comprising a ionic component.

16. The composition of item 15, wherein the ionic component has a concentration of 0.01% (w/v) to 10% (w/v), such as of 0.5% (w/v) to 10% (w/v), more in particular of 1% (w/v) to 5% (w/v).

17. The composition of item 15, wherein the ionic component has a concentration of 0.1 mM to 100 mM, such as 1 mM to 30 mM, more in particular of 5 mM to 15 mM.

18. The composition of any one of items 15 to 17, wherein the ionic component is selected from: salts, acids, or bases comprising Mg²⁺, Na⁺, K⁺, Cl⁻, HCO₃⁻, or a combination thereof.

19. The composition of any one of items 15 to 17, wherein the ionic component is selected from: MgCl₂. MgSO₄, KCl, KH₂PO₄, NaHCO₃, Na₂HPO₄, C₂H₂MgO₄ (magnesium formate), C₃H₃NaO₃ (sodium pyruvate), C₂H₃NaO₂ (sodium acetate), or a combination thereof.

20. The composition of any one of items 15 to 17, wherein the ionic component is an organic anion selected from: citrate, pyruvate, malate, oxaloacetate, formate, glutamate, α-ketoglutarate, succinate, acetate anions, or a combination thereof.

21. The composition of any one of items 1 to 20, further comprising albumin at a concentration of 0.01% (w/v) to 10% (w/v), such as 0.1% (w/v) to 5% (w/v).

22. The composition of any one of items 1 to 21, wherein the albumin is bovine serum albumin (BSA), human serum albumin (HSA), or a combination thereof.

23. The composition of item 21 or 22, wherein the albumin is BSA at a concentration of 0.05% (w/v) to 3% (w/v), such as 0.1% (w/v).

24. The composition of any one of items 1 to 3, wherein (i) the buffer has pH 7.4 and comprises 20 mM tris-(hydroxymethyl)aminomethane hydrochloride) (Tris), 2 mM 2,2′,2″,2′″-(Ethane-1,2-diyldinitrilo)-tetraacetic acid (EDTA), and 10 mM MgCl₂.

25. The composition of any one of items 1 to 3, wherein (i) the buffer has pH 7.25 and comprises 5 mM 4-morpholinepropanesulfonic acid (MOPS), 10 mM 1,2-bis(o-aminophenoxy)ethane-N,N,N′,N′-tetraacetic acid (BAPTA), and 5 mM sodium pyruvate.

26. The composition of any one of items 1 to 3, wherein (i) the buffer has pH 7.2 and comprises 10 mM 2-[4-(2-hydroxyethyl)-piperazin-1-yl]-ethanesulfonic acid (HEPES), and 1 mM ethylene glycol-bis(β-aminoethyl ether)-N,N,N′,N′-tetraacetic acid (EGTA).

27. The composition of any one of items 1 to 26, wherein the composition includes less than a cryopreservative amount of propylene glycol, ethylene glycol, glycerol and dimethyl sulfoxide (DMSO), or no propylene glycol, ethylene glycol, glycerol and dimethyl sulfoxide (DMSO).

28. The composition of any one of items 1 to 26, wherein the composition includes less than a cryopreservative amount of dimethyl sulfoxide (DMSO) or no dimethyl sulfoxide (DMSO).

29. The composition of any one of items 1 to 28, wherein (ii) the trehalose has a concentration of no more than 1500 mM, such as no more than 1300 mM.

30. The composition of item 29, wherein (ii) the trehalose has a concentration of no more than 500 mM, preferably of no more than 450 mM.

31. The composition of any one of items 1 to 29, wherein (ii) the trehalose has a concentration of at least 250 mM, such as 300 mM.

32. The composition of any one of items 1 to 29, wherein (ii) the trehalose has a concentration of 150 mM to 1000 mM, such as of 200 mM to 600 mM.

33. The composition of any one of items 1, 2, or 4 to 32, wherein (a) the amino acid(s) is selected from leucine, isoleucine (e.g., L-isoleucine), proline (e.g., L-proline), methylproline, benzylproline, hydroxyproline, aminoproline, dehydroproline, aziridinecarboxylic acid, azetidinecarboxylic acid, pipecolic acid, oxaproline, thiaproline, valine (e.g., L-valine), alanine (e.g., L-alanine), glycine, asparagine (e.g., L-asparagine), aspartic acid (e.g., L-aspartic acid), glutamic acid (e.g., L-glutamic acid), serine (e.g., L-serine), histidine (e.g., L-histidine), cysteine (e.g., L-cysteine), tryptophan (e.g., L-tryptophan), tyrosine (e.g., L-tyrosine), arginine (e.g., L-arginine), glutamine (e.g., L-glutamine), lysin, threonine, selenocysteine, methionine, phenylalanine, creatine (e.g., L-creatine), taurine (e.g., L-taurine), betaine, ectoine, dimethylglycine, ethylmethylglycine, an RGD peptide, or a combination thereof.

34. The composition of item 33, wherein (a) the amino acid(s) is selected from leucine, isoleucine (e.g., L-isoleucine), valine (e.g., L-valine), alanine (e.g., L-alanine), glycine, asparagine (e.g., L-asparagine), aspartic acid (e.g., L-aspartic acid), glutamic acid (e.g., L-glutamic acid), serine (e.g., L-serine), histidine (e.g., L-histidine), cysteine (e.g., L-cysteine), tryptophan (e.g., L-tryptophan), tyrosine (e.g., L-tyrosine), arginine (e.g., L-arginine), glutamine (e.g., L-glutamine), or a combination thereof.

35. The composition of item 33, wherein (a) the amino acid(s) is selected from methylproline, benzylproline, hydroxyproline, aminoproline, dehydroproline, aziridinecarboxylic acid, azetidinecarboxylic acid, pipecolic acid, oxaproline, thiaproline, or a combination thereof.

36. The composition of item 33, wherein (a) the amino acid is proline, such as L-proline.

37. The composition of any one of items 1, 2, or 4 to 36, wherein (a) the amino acid(s) has a concentration of at least 160 mM, such as of at least 180 mM.

38. The composition of any one of items 1, 2, or 4 to 37, wherein (a) the amino acid(s) has a concentration of at least 200 mM, such as of at least 250 mM.

39. The composition of any one of items 1, 2, or 4 to 38, wherein (a) the amino acid(s) has a concentration of at least 300 mM, such as of at least 500 mM.

40. The composition of any one of items 1, 2, or 4 to 39, wherein (a) the amino acid(s) has a concentration of at least 800 mM, such as of at least 1000 mM.

41. The composition of any one of items 1, 2, or 4 to 40, wherein (a) the amino acid(s) has a concentration of at least 1100 mM, such as of at least 1200 mM.

42. The composition of any one of items 1, 2, or 4 to 41, wherein (a) the amino acid(s) has a concentration of at least 1300 mM, such as of at least 1500 mM.

43. The composition of any one of items 1, 2, or 4 to 42, wherein (a) the amino acid(s) has a concentration of at least 1800 mM, such as 2000 mM.

44. The composition of any one of items 1, 2, or 4 to 43, wherein (b) the sugar(s) is selected from: mono-, di-, or trisaccharide, or a combination thereof.

45. The composition of any one of items 1, 2, or 4 to 44, wherein (b) the sugar(s) is selected from maltose, lactose, fructose, sucrose, glucose, dextran, melezitose, raffinose, nigerotriose, maltotriose, maltotriulose, kestose, cellobiose, chitobiose, lactulose, or a combination thereof.

46. The composition of any one of items 1, 2, or 4 to 45, wherein (b) the sugar(s) is selected from sucrose, glucose, or a combination thereof.

47. The composition of item 46, wherein (b) the sugar(s) is a combination of glucose and sucrose.

48. The composition of item 46, wherein (b) the sugar(s) is glucose.

49. The composition of item 46, wherein (b) the sugar(s) is sucrose.

50. The composition of any one of items 1, 2, or 4 to 49, wherein (b) the sugar(s) has a concentration of at least 160 mM, such as of at least 180 mM.

51. The composition of item 50, wherein (b) the sugar(s) has a concentration of at least 200 mM, such as of at least 250 mM.

52. The composition of any one of items 50 or 51, wherein (b) the sugar(s) has a concentration of at least 300 mM, such as of at least 330 mM.

53. The composition of any one of items 1, 2, or 4 to 52, wherein (b) the sugar(s) has a concentration of no more than 2000 mM, such as no more than 1800 mM, more in particular of no more than no more than 1500 mM.

54. The composition of item 53, wherein (b) the sugar(s) has a concentration of no more than 1400 mM such as, no more than 1200 mM, more in particular no more than 1000 mM.

55. The composition of any one of items 1 to 54, wherein (c) the polymer is a biocompatible, hydrophilic, amphiphilic polymer, or a combination thereof.

56. The composition of any one of items 1 to 55, wherein (c) the polymer(s) is selected from: poloxamer, such poloxamer 142, poloxamer 188, poloxamer 331, or poloxamer 407, alginate, polyethylene glycol (PEG), such as PEG400 or PEG1000, polyglutamic acid, polyvinyl alcohol, polyvinyl pyrrolidone, or a combination thereof.

57. The composition of any one of items 1 to 56, wherein (c) the polymer(s) is polyethylene glycol (PEG).

58. The composition of any one of items 1 or 2, wherein (c) the polymer(s) has a concentration of 2.5% (w/v) to 25% (w/v), such as 5% (w/v) to 25% (w/v).

59. The composition of any one of items 1, 2, or 55 to 58, wherein (c) the polymer(s) has a concentration of 10% (w/v) to 25% (w/v), such as 15% (w/v) to 20% (w/v).

60. The composition of any one of items 1, 4 to 33, or 36 to 59, wherein
(iii) the first cryoprotecting agent is (a) an amino acid, wherein the amino acid is proline, such as L-proline, at a concentration of at least 150 mM, such as of at least 200 mM, preferably of at least 300 mM; and
(iv) the second cryoprotecting agent(s) is selected from one or more (b) sugar(s), (c) polymer(s), or a combination thereof, wherein
   (b) the sugar(s) is glucose, sucrose, or a combination thereof, and has a total concentration of at least 150 mM, such as of at least 200 mM, more in particular of at least 300 mM, such as of at least 500 mM; and
   (c) the polymer is polyethylene glycol (PEG) at a concentration of 2.5% (w/v) to 30% (w/v), such as 5% (w/v) to 25% (w/v), more in particular of 10% (w/v) to 20% (w/v), such as 15% (w/v).

61. The composition of any one of items 1, 4 to 33, 35, or 37 to 59, wherein
(iii) the first cryoprotecting agent(s) is one or more (a) an amino acid(s), wherein the amino acid is a proline derivative selected from methylproline, benzylproline, hydroxyproline, aminoproline, dehydroproline, aziridinecarboxylic acid, azetidinecarboxylic acid, pipecolic acid, oxaproline, thiaproline, or a combination thereof, at a concentration of at least 150 mM, such as of at least 200 mM, preferably of at least 300 mM; and
(iv) the second cryoprotecting agent(s) is selected from one or more (b) sugar(s), (c) polymer(s), or a combination thereof, wherein
   (b) the sugar(s) is glucose, sucrose, or a combination thereof, and has a total concentration of at least 150 mM, such as of at least 200 mM, more in particular of at least 300 mM, such as of at least 500 mM; and
   (c) the polymer is polyethylene glycol (PEG) at a concentration of 2.5% (w/v) to 30% (w/v), such as 5% (w/v) to 25% (w/v), more in particular of 10% (w/v) to 20% (w/v), such as 15% (w/v).

62. The composition of any one of items 2, or 4 to 59, wherein
(iii) the first cryoprotecting agent(s) is one or more (b) sugar(s), wherein the sugar(s) is glucose, sucrose, or a combination thereof, at a total concentration of at least 160 mM, such as of at least 200 mM, more in particular of at least 300 mM, such as of at least 500 mM; and
(iv) the second cryoprotecting agent(s) is selected from one or more (a) amino acid(s), (c) polymer(s), or a combination thereof, wherein
   (a) the amino acid(s) is selected from the amino acid(s) according to any one of items 33 to 36 at a concentration of at least 150 mM, such as of at least 200 mM, preferably of at least 300 mM;
   (b) the polymer is polyethylene glycol (PEG) at a concentration of 2.5% (w/v) to 30% (w/v), such as 5% (w/v) to 25% (w/v), more in particular of 10% (w/v) to 20% (w/v), such as 15% (w/v).

63. The composition of item 62, wherein (a) the amino acid is proline, such as L-proline.

64. The composition of any one of items 3 to 32, or 55 to 57, wherein
(iii) the cryoprotecting agent is (c) a polymer(s), wherein the polymer(s) is polyethylene glycol (PEG) at a concentration of 16% (w/v) to 30% (w/v), such as 20% (w/v) to 30% (w/v), such as 25% (w/v).

65. The composition according to any one of items 60, 62 or 63, wherein
(a) the amino acid is proline, such as L-proline, at a concentration of at least 500 mM, such as of at least 600 mM, in particular of at least 800 mM.

66. The composition of any one of items 60, 62, or 63, wherein
the amino acid is proline, such as L-proline, at a concentration of at least 1000 mM, such as 1200 mM.

67. The composition of any one of items 60, 62, or 63, wherein
the amino acid is proline, such as L-proline, at a concentration of at least 1300 mM, such as of at least 1500 mM.

68. The composition of any one of items 60, 62, or 63, wherein
the amino acid is proline, such as L-proline, at a concentration of at least 1600 mM, such as of at least 1800 mM, in particular of at least 2000 mM.

69. The composition of any one of items 60, 62, 63 or 65 to 68, wherein
the amino acid is proline, such as L-proline; and
the sugar is glucose at a concentration of 200 mM to 1300 mM, such as of 300 mM to 1200 mM, in particular of 350 mM to 1100 mM.

70. The composition of any one of items 60, 62, 63, or 65 to 68, wherein
the amino acid is proline, such as L-proline; and
the sugar is sucrose at a concentration of 160 mM to 900 mM, such as of 200 mM to 800 mM, in particular 250 mM to 650 mM.

71. The composition of any one of items 60, 62, 63 or 65 to 68, wherein
the amino acid is proline, such as L-proline; and
the polymer is polyethylene glycol (PEG) at a concentration of 5% (w/v) to 30% (w/v), 10% (w/v) to 20% (w/v), such as 15% (w/v).

72. The composition of any one of items 1, 4 to 33, 36, or 60, wherein the cryoprotecting agent consists of proline, such as L-proline, at a concentration according to any one of items 37 to 43.

73. The composition of item 72, wherein proline, such as L-proline, is at a concentration of 600 mM or 1200 mM.

74. The composition of any one of items 1, 4 to 32, 35 or 61, wherein the cryoprotecting agent consists of one or more (a) amino acid(s), wherein (a) the amino acid is a proline derivative selected from methylproline, benzylproline, hydroxyproline, aminoproline, dehydroproline, aziridinecarboxylic acid, azetidinecarboxylic acid, pipecolic acid, oxaproline, thiaproline, or a combination thereof, at a total concentration of at least 200 mM, preferably of at least 300 mM, more preferably of at least 500 mM.

75. The composition of any one of items 1, 4 to 32, 35, 61, or 74 wherein the cryoprotecting agent consists of one or more (a) amino acid(s), wherein
the amino acid is a proline derivative selected from: methylproline, benzylproline, hydroxyproline, aminoproline, dehydroproline, aziridinecarboxylic acid, azetidinecarboxylic acid, pipecolic acid, oxaproline, thiaproline, or a combination thereof, at a concentration of at a concentration of at least 400 mM, preferably of at least 600 mM, such as of at least 800 mM.

76. The composition of any one of items 1, 4 to 32, 35, 61, 74, or 75, wherein the cryoprotecting agent consists of one or more (a) amino acid(s), wherein
the amino acid is a proline derivative selected from: methylproline, benzylproline, hydroxyproline, aminoproline, dehydroproline, aziridinecarboxylic acid, azetidinecarboxylic acid, pipecolic acid, oxaproline, thiaproline, or a combination thereof, at a concentration of at least 1000 mM, such as of at least 1200 mM, in particular of at least 1500 mM, such as 2000 mM.

77. The composition of any one of items 2, 4 to 32, or 62, wherein the cryoprotecting agent(s) consists of glucose, sucrose, or a combination thereof, at a total concentration of at least 200 mM, such as of at least 300 mM, more in particular of at least 500 mM, and no more than 2000 mM, such as no more than 1700 mM.

78. The composition of any one of items 2, 4 to 32, or 62, wherein the cryoprotecting agent(s) consists of glucose, sucrose, or a combination thereof, at a total concentration of at least 550 mM, such as of at least 650 mM, more in particular of at least 800 mM, and no more than 2000 mM, such as no more than 1650 mM.

79. The composition of any one of items 2, 4 to 32, or 62, wherein the cryoprotecting agent(s) consists of glucose, sucrose, or a combination thereof, at a total concentration of at least 1000 mM, such as of at least 1200 mM, in particular of at least 1350 mM, and no more than 2000 mM, such as no more than 1650 mM.

80. The composition of any one of items 2, 4 to 32, or 62, wherein the cryoprotecting agent consists of glucose at a total concentration of at least 300 mM, such as of at least 500 mM, in particular of at least 800 mM, but no more than 1200 mM, such as no more than 1000 mM.

81. The composition of any one of items 2, 4 to 32, or 62, wherein the cryoprotecting agent consists of sucrose at a total concentration of at least 200 mM, such as of at least 250 mM, in particular of at least 300 mM, but no more than 800 mM, such as no more than 650 mM.

82. The composition of any one of items 1 to 81, further comprising isolated mitochondria, such as isolated viable mitochondria.

83. The composition of item 82, wherein the isolated mitochondria are mammalian isolated mitochondria, such as mammalian isolated viable mitochondria.

84. The composition of item 82, wherein the isolated mitochondria are human isolated mitochondria, such as human isolated viable mitochondria.

85. The composition of any one of items 82 to 84, wherein the mitochondria have been isolated from cells, tissues, or organs.

86. The composition of item 85, wherein the tissue is liver tissue, skeletal muscle, heart, brain, kidney, placenta, lung tissue, or adipose tissue.

87. The composition of item 86, wherein the mitochondria have been isolated from cultured cells, such as cultured human cells.

88. The composition of any one of items 85 or 87, wherein the mitochondria have been isolated from cells selected from the group consisting of placenta cells, muscle tissue cells, cardiac fibroblasts, HeLa cells, prostate cancer cells, yeast, or any mixture thereof.

89. The composition of any one of items 82 to 88, wherein the isolated mitochondria, have a concentration of at least 0.02 μg/μL, such as at least 0.05 μg/μL, in particular of at least 0.1 μg/μL.

90. The composition of item 89, wherein the isolated mitochondria have a concentration of at least 0.2 μg/μL, such as of at least 0.5 μg/μL, in particular of at least 0.75 μg/μL.

91. The composition of any one of items 89 or 90, wherein the isolated mitochondria have a concentration of at least 1.0 μg/μL, such as at least 1.5 μg/μL, in particular of at least 2 μg/μL, such as 2.5 μg/μL.

92. The composition of any one of items 89 to 91, wherein the isolated mitochondria have a concentration of at least 5 μg/μL, such as of at least 10 μg/μL, such as 20 μg/μL or 30 μg/μL.

93. The composition of anyone of items 82 to 92, wherein the isolated mitochondria have a concentration of no more than 100 μg/μL, such as no more than 80 μg/μL, in particular no more than 50 μg/μL.

94. The composition of anyone of item 82 to 93, wherein the isolated mitochondria have a concentration of no more than 25 μg/μL, such as no more than 15 μg/μL, in particular no more than 10 μg/μL, such as no more than 5 μg/μL.

95. The composition of any one of items 82 to 94, wherein the isolate mitochondria are linked to a pharmaceutical agent, diagnostic agent, imaging agent, therapeutic agent, or any other biocompatible agent.

96. The composition of any one of items 82 to 94, wherein the mitochondria are linked to an antibody.

97. The composition of any one of items 82 to 94, wherein the mitochondria are linked to an antigen binding fragment, wherein the antigen binding fragment is at least one portion of an antibody or TCR, or recombinant variants thereof.

98. The composition of any one of items 95 to 97, wherein the mitochondria are linked to the agent, the antibody, or the antigen binding fragment, by a covalent bond.

99. The composition of any one of items 95 to 97, wherein the mitochondria are linked to the agent, the antibody, or the antigen, by a non-covalent bond, such as an electrostatic bond.

100. The composition of any one of items 95 to 97, wherein the agent, the antibody, or the antigen, are embedded in the mitochondria, embedded in the mitochondrial membrane, substantially enclosed within a mitochondrion, or encapsulated entirely by mitochondria.

101. The composition of any one of items 95 to 99, wherein are the agent, the antibody, or the antigen, are linked to the outer membrane of mitochondria by a covalent or a non-covalent bond, such as an electrostatic bond.

102. The composition of any one of items 82 to 101, wherein the isolated mitochondria are mitochondria modified by gene editing.

103. The composition of any one of items 82 to 101, wherein the isolated mitochondria comprise exogenous mtDNA.

104. A method for the cryopreservation of a composition comprising isolated mitochondria, such as isolated viable mitochondria, according to any one of the preceding items, the method comprising the steps of:
(a) freezing the composition at a temperature below 0° C.; and
(b) storing the frozen composition obtained according to step (a) at a temperature below 0° C., such as −4° C., preferably −20° C.

105. The method of item 104, wherein the method further comprises a further step (c) subsequent to step (b), consisting in thawing the frozen composition at a temperature above 0° C.

106. The method of any one of items 104 or 105, wherein
(a) freezing is at a temperature below −20° C., such as below −50° C., preferably at a temperature below 70° C.

107. The method of any one of items 104 or 105, wherein
(a) freezing is at a temperature below −100° C., such as below −150° C., in particular below −180° C.

108. The method of any one of items 104 or 105, wherein
(a) freezing is done in liquid nitrogen at a temperature of −196° C. or in dry ice at a temperature of −78.5° C., preferably in dry ice at −78.5° C.

109. The method of any one of items 104 or 105, wherein 5
(a) freezing is snap-freezing.

110. The method of any one of items 104 or 105, wherein
(a) freezing is a gradual freezing at a rate of at least 5° C./min, such as of at least 10° C./min, such as 20° C./min or 30° C./minute.

111. The method of any one of items 104 to 110, wherein
(b) storing has a duration of a period of at least 24 hours, such as of 120 hours prior to thawing, preferably wherein
(b) storing has a duration of a period of at least 1 week, such as 4 weeks.

112. The method of any one of items 104 to 111, wherein
(c) thawing is performed at a temperature higher than 4° C. and lower than 40° C., such as at a temperature between 20° C. and 38° C.

113. A composition comprising isolated mitochondria, such as isolated viable mitochondria, according to any one of the preceding items or a composition prepared by the method of any one of items 104 to 112, in a therapeutically effective amount for use in the treatment of a disease, such as by therapeutic mitochondrial transplantation (TMT), in a subject in need thereof.

114. The composition according to item 113 for use in the treatment of a mitochondrial or mitochondrial-related disease, in a subject in need thereof.

115. The composition according to item 113 for use in the treatment of cancer or tumor in a subject in need thereof.

116. The composition according to item 113 for use in the treatment of autoimmune disease.

117. The composition according to item 113 for use in the treatment of ischemia related injuries, such as lung-, kidney-, cardiac-, or brain-ischemia-reperfusion injuries.

120. The composition according to item 114 for use in the removal of blockages in the blood vessels, such as a blood clot.

121. The composition according to item 114 for use in gene therapies, in particular for use in gene therapy for the treatment of cancer, infectious diseases, or autoimmune diseases.

122. The composition according to item 114 for use in the transplantation of the mitochondria, such as isolated viable mitochondria, into immune cells to produce mitochondria-enhanced immune effector cells, such as, but not limited to, chimeric antigen receptor (CAR) T-cell, CAR-NK cell, CAR-macrophage, neutrophil, tumor-infiltrating lymphocyte (TIL), gamma-delta T cell.

123. The composition according to item 114 for use in the treatment of a disease in a subject in need, wherein said composition is to be administered to a subject in need by topical or parental administration.

124. The composition according to item 114 for use in the treatment of a disease in a subject in need, wherein said composition is to be administered to a subject in need in the form of an aerosol.

125. The composition according to item 114 for use in the treatment of a disease in a subject in need, wherein said composition is to be administered to a subject in need by direct injection into a blood vessel, a tissue, or an organ.

126. The composition according to any one of the preceding items for use in the treatment of a disease in a subject in need thereof, wherein said composition is to be delivered to the target cells in vitro of a subject in need or of a donor.

127. The composition according to any one of the preceding items, wherein the viable mitochondria comprised in the composition are autologous, allogeneic, or xenogeneic.

128. The composition according to any one of items 114 to 127, wherein the composition has undergone a cycle of freeze-thaw, such as a freeze-thaw cycle according to the method of any one of items 106 to 114, prior to use in the treatment of the disease.

129. Use of the composition of any one of the preceding items for the cryopreservation of viable mitochondria, such as isolated viable mitochondria.

130. Use of a composition prepared by the method of any one of items 105 to 113 for the cryopreservation of viable mitochondria, such as isolated viable mitochondria.

This invention provides new and efficient compositions and methods for cryopreserving isolated mitochondria, such as isolated viable mitochondria. This invention further provides a cryopreserved composition comprising isolated mitochondria, e.g., isolated viable mitochondria. The cryopreserved mitochondria of the invention may be utilized in different types of therapies, such as, for example, in cell and gene therapies after thawing. These therapies need consistent and reproducible results with high mitochondria viability and functionality for therapeutic effectiveness. Mitochondria damaging during cryopreservation and thaw may render the therapy ineffective. Mitochondria are particularly sensitive to stress of freezing and thawing and lack of an optimized cryopreservation method has hindered research and medical applications. As of today, there is a strong felt need for cell and gene therapies as well as for therapeutic mitochondria transplantation (TMT), which can rely on cryopreservation methods and compositions to preserve mitochondria safely and efficiently during the manufacturing process, storage, and transport. Till the present, in basic research and in the medical applications (e.g., in therapeutics, diagnostics and imaging) there is still a felt hanker after ready-to use isolated mitochondria, which maintain their integrity and viability during the freezing process, storage at low temperatures as well as during the thawing process prior to use. Thus, the technical problem underlying the present invention is to provide new and innovative methods and compositions suitable for the cryopreservation of isolated viable mitochondria, which allow the maintenance of their structural integrity and/or functional properties. The present invention provides cryopreserved compositions comprising isolated viable mitochondria, which remain viable, respiration-competent and/or integer, during storage at low temperature and during freeze-thaw cycles, preferably the mitochondria remain viable, respiration-competent and integer. The cryopreserved mitochondria of the present invention show structural as well as functional properties, which are comparable to the ones possessed by freshly isolated mitochondria and advantageously exhibit a long shelf-life time. The compositions of the invention are non-toxic, safe and physiologically compatible. Therefore, they can be used in-vivo.

### SUMMARY

The present invention relates to a composition comprising (i) an aqueous buffer having a pH of 5.5 to 8.5; (ii) trehalose at a concentration of at least 150 mM; (iii) a first cryoprotecting agent(s) selected from one or more (a) amino acid(s) at a total concentration of at least 150 mM; and optionally (iv) a second cryoprotecting agent(s) selected from one or more (b) sugar(s), (c) polymer(s), or a combination thereof, wherein (b) the sugar(s) has a total concentration of at least 150 mM; and (c) the polymer(s) has a total concentration of 2.5% (w/v) to 30% (w/v). In the composition of the invention, it may be that (iii) the first cryoprotecting agent is (a) an amino acid, wherein the amino acid is proline, such as L-proline, at a concentration of at least 150 mM, such as of at least 200 mM, preferably of at least 300 mM; and (iv) the second cryoprotecting agent(s) is selected from one or more (b) sugar(s), (c) polymer(s), or a combination thereof, wherein (b) the sugar(s) is glucose, sucrose, or a combination thereof, and has a total concentration of at least 150 mM, such as of at least 200 mM, more in particular of at least 300 mM, such as of at least 500 mM; and (c) the polymer is polyethylene glycol (PEG) at a concentration of 2.5% (w/v) to 30% (w/v), such as 5% (w/v) to 25% (w/v), more in particular of 10% (w/v) to 20% (w/v), such as 15% (w/v). It may also be that (iii) the first cryoprotecting agent(s) is one or more (a) an amino acid(s), wherein the amino acid is a proline derivative selected from methylproline, benzylproline, hydroxyproline, aminoproline, dehydroproline, aziridinecarboxylic acid, azetidinecarboxylic acid, pipecolic acid, oxaproline, thiaproline, or a combination thereof, at a concentration of at least 150 mM, such as of at least 200 mM, preferably of at least 300 mM; and (iv) the second cryoprotecting agent(s) is selected from one or more (b) sugar(s), (c) polymer(s), or a combination thereof, wherein (b) the sugar(s) is glucose, sucrose, or a combination thereof, and has a total concentration of at least 150 mM, such as of at least 200 mM, more in particular of at least 300 mM, such as of at least 500 mM; and (c) the polymer is polyethylene glycol (PEG) at a concentration of 2.5% (w/v) to 30% (w/v), such as 5% (w/v) to 25% (w/v), more in particular of 10% (w/v) to 20% (w/v), such as 15% (w/v).

The invention in a further embodiment relates to a composition comprising (i) an aqueous buffer having a pH of 5.5 to 8.5; (ii) trehalose at a concentration of at least 150 mM; (iii) a first cryoprotecting agent(s) selected from one or more (b) sugar(s) at a total concentration of at least 160 mM; and optionally (iv) a second cryoprotecting agent(s) selected from (a) amino acid(s), (c) polymer(s), or a combination thereof, wherein (a) the amino acid has a total concentration of at least 150 mM; and (c) the polymer has a total concentration of 2.5% (w/v) to 30% (w/v). It may be that in the composition (iii) the first cryoprotecting agent(s) is one or more (b) sugar(s), wherein the sugar(s) is glucose, sucrose, or a combination thereof, at a total concentration of at least 160 mM, such as of at least 200 mM, more in particular of at least 300 mM, such as of at least 500 mM; and (iv) the second cryoprotecting agent(s) is selected from one or more (a) amino acid(s), (c) polymer(s), or a combination thereof, wherein (a) the amino acid(s) is selected from the amino acid(s) according to any one of items 33 to 36 at a concentration of at least 150 mM, such as of at least 200 mM, preferably of at least 300 mM; (b) the polymer is polyethylene glycol (PEG) at a concentration of 2.5% (w/v) to 30% (w/v), such as 5% (w/v) to 25% (w/v), more in particular of 10% (w/v) to 20% (w/v), such as 15% (w/v). It may also be that (a) the amino acid is proline, such as L-proline.

The invention in a further embodiment relates to a composition comprising (i) an aqueous buffer having a pH of 5.5 to 8.5; (ii) trehalose at a concentration of at least 150 mM; and (iii) a cryoprotecting agent(s) selected from one or more (c) polymer(s), wherein the total concentration of the polymer(s) is from 16% (w/v) to 30% (w/v). It may be that (iii) the cryoprotecting agent is (c) a polymer(s), wherein the polymer(s) is polyethylene glycol (PEG) at a concentration of 16% (w/v) to 30% (w/v), such as 20% (w/v) to 30% (w/v), such as 25% (w/v).

The present invention relates to a cryopreservative composition comprising: (i) an aqueous buffer having a pH of 5.5 to 8.5; (ii) trehalose at a concentration of at least 150 mM; (iii) a cryoprotecting agent selected from the group consisting of (a) amino acid, (b) sugar, (c) polymer, or a combination thereof, wherein: the amino acid has a concentration of at least 150 mM; the sugar has a concentration of at least 150 mM, such as of at least 160 mM; and the polymer has a concentration of 2.5% (w/v) to 30% (w/v), such as of 16% (w/v) to 30% (w/v).

The present invention provides a cryopreservative composition comprising: (i) the aqueous buffer has a pH of 5.5 to 8.5; (ii) trehalose has a concentration of at least 150 mM; (iii) the cryoprotecting agent is selected from the group consisting of (a) amino acid, (b) sugar, (c) polymer, or a combination thereof, wherein: the amino acid is proline, such as L-proline, at a concentration of at least 150 mM; the sugar is glucose, sucrose, or a combination thereof, and has a total concentration of at least 150 mM, such as of at least 160 mM; and the polymer is polyethylene glycol (PEG) at a concentration of 2.5% (w/v) to 30% (w/v), such as of 16% (w/v) to 30% (w/v).

The cryopreservative composition of the invention further comprises isolated mitochondria, e.g., isolated viable mitochondria, in a therapeutically effective amount for use in treating conditions, which benefit from increased mitochondrial function, in a subject in need thereof.

The present invention further provides a method suitable for the cryopreservation of the composition comprising the isolated mitochondria, e.g., isolated viable mitochondria, the method comprising the steps of: (a) freezing the composition at a temperature below 0° C.; and (b) storing the frozen composition obtained according to step (a) at a temperature below 0° C.

Within the invention, the mitochondria may also be subject to lyophilization and/or spray-freezing, such as spray-freeze-drying or spray-freeze-lyophilizing

### BRIEF DESCRIPTION OF THE DRAWINGS

FIG. 1. Mitochondria frozen in sucrose-based buffer do not maintain inner and outer membrane integrity.

The isolated mitochondria of FIG. 1A, FIG. 1B, FIG. 1C and FIG. 1D were suspended in a aqueous buffer at pH 7.2 (10 mM HEPES adjusted to pH 7.2 with KOH), 1 mM EGTA, and 300 mM sucrose. The isolated mitochondria of FIG. 1E, FIG. 1F and FIG. 1G were suspended in two different buffers (both at pH 7.2): a “trehalose-based buffer” (10 mM HEPES, 1 mM EGTA, 300 mM trehalose) and a “sucrose-based buffer” (10 mM HEPES, 1 mM EGTA, 300 mM sucrose).

FIG. 1A. Isolated mitochondria were frozen in liquid nitrogen (LN), thawed in a 37° C. water bath, stained with MitoTrackers Red and Green and imaged using a fluorescent microscope. FIG. 1B. The same procedure as described for FIG. 1A was followed, but mitochondria were frozen on dry ice rather than in liquid nitrogen. FIG. 1C. Quantification of the MitoTracker Red signal of the stained mitochondria of FIG. 1A (n=3). FIG. 1D. Quantification of the MitoTracker Red signal of the stained mitochondria of FIG. 1B (n=3). FIG. 1E. Mitochondria were isolated either in sucrose- or trehalose-based isolation buffer, snap-frozen in liquid nitrogen (LN) or kept on ice (non-frozen), thawed in a 37° C. water bath, and the ATP content was measured (n=3). FIG. 1F. The same procedure as described for FIG. 1E was followed, but mitochondria were frozen on dry ice (n=3). FIG. 1G. Mitochondria isolated in either sucrose- or trehalose-based isolation buffer were frozen on dry ice, thawed in a 37° C. water bath, incubated for 15 minutes at 37° C. after thawing, and pelleted to separate cytochrome C released into supernatant (indicated as S in FIG. 1G) from mitochondrial pellet-associated cytochrome C (indicated as P in FIG. 1G). The samples were further analyzed by acrylamide gel electrophoresis and western blotting using anti-cytochrome C antibody. All data are presented as mean±SD (Standard Deviation).

FIG. 2. Freezing of mitochondria in trehalose-based buffer is not sufficient to maintain their membrane potential and ATP content.

FIG. 2A. Mitochondria were isolated in a trehalose-based isolation buffer and frozen-thawed on dry ice/37° C. water bath, for a number of cycles as indicated in FIG. 2A (e.g., from 1 to 5 cycles). The non-frozen mitochondria were simply kept on ice, at 4° C. ca (“non-frozen” in FIG. 2A-2G). After staining with MitoTrackers Red and Green, mitochondria were imaged using automated fluorescent microscope. FIG. 2B. Quantification of the MitoTracker Red signal of the sample shown in FIG. 2A (n=3). FIG. 2C. ATP content of mitochondria frozen as described in FIG. 2A (n=3). FIG. 2D. Mitochondria were frozen on dry ice, in liquid nitrogen (LN), or first frozen on dry ice and then placed on liquid nitrogen (Dry ice+LN). Membrane potential was determined by MitoTracker Red and Green staining, as described for FIG. 1A. Overlay images from MitoTracker Red and Green staining are presented. FIG. 2E. Quantification of MitoTracker Red signal (MTR) from stained mitochondria of the samples of FIG. 2D (n=3). FIG. 2F. ATP content of mitochondria treated as described for FIG. 2D (n=3). FIG. 2G. Isolated mitochondria were frozen on dry ice and thawed either in a water bath (37° C.), at room temperature (20° C.), or on ice (4° C.). ATP content was measured and compared to non-frozen mitochondria incubated on ice. All data are presented as mean±SD.

FIG. 3. Addition of certain cryoprotectants to freezing buffer prevents mitochondrial damage in a concentration-dependent manner.

FIG. 3A. Isolated mitochondria were suspended in an aqueous buffer containing 10 mM HEPES (adjusted at pH 7.2 with KOH), 300 mM trehalose and 1 mM EGTA. Each sample containing a cryoprotectant was prepared by adding to said mitochondrial suspension one of the six different cryoprotectants (i.e., CRYO6, CRYO12, CRYO15, CYRO22, CRYO25, or CRYO33; the details of each cryoprotectant are provided in Table 1b) at different concentration, respectively at concentration of 5%, 10%, 15%, 20%, 25%, or 30% volume of cryoprotectant over the final total volume of the suspension (i.e., % (v_{CRYO}/vₜₒₜ)). For instance, a suspension (e.g., composition) comprising 20% (v/v) of CRYO6, was prepared by adding 10 μL of a 6 M proline solution (i.e., CRYO6) in 40 μL of the suspension containing 300 mM trehalose, 1 mM EGTA, 10 mM HEPES (pH 7.2), and mitochondria. The concentration of mitochondria was of 10 μg/50 μL for the samples used in the ATP assay measurements and 15 μg/20 μL for the microscopy analysis. One of the samples contained no cryoprotectant (i.e., 0% of the final volume of the suspension). As control, it was used a suspension of freshly isolated mitochondria, which was not frozen (“Fresh”). The samples were first (1) frozen; subsequently (2) thawed, and then (3) stained with MitoTrackers Red and Green and imaged with an automated fluorescent microscope. The MitoTracker Red signal was calculated relative to non-frozen mitochondria stored on ice (n=3). The average values are presented as a heat map. FIG. 3B. Representative images of isolated mitochondria frozen with indicated cryoprotectants and stained with MitoTrackers Red and Green, along with corresponding controls (non-frozen, freshly isolated mitochondria and frozen mitochondria without cryoprotectants). FIG. 3C. ATP content of isolated mitochondria, which have been frozen in the presence of indicated concentrations of selected cryoprotectants, relative to ATP content of non-frozen (fresh) isolated mitochondria.

FIG. 4. Viability of frozen-thawed mitochondria comprised in a composition containing two cryoprotectants

FIG. 4A. Freshly isolated mitochondria were kept on ice (indicated as “Fresh” in the graph), frozen without cryoprotectants (indicated as “Frozen” in the graph), or frozen in the presence of indicated concentrations of either one or two cryoprotectants (indicated in the graphs by their respective concentration (% volume of the cryoprotectant/total volume of the suspension) and type of cryoprotectant utilized). ATP content of mitochondria was measured after thawing and normalized to non-frozen mitochondria.

FIG. 5. Freezing rate affects viability of mitochondria contained in a composition comprising a cryoprotectant

FIG. 5A. Mitochondria were frozen at controlled flow rates of 2 K/min, 6 K/min and 10 K/min, stored at −80° C. for 1 month and compared to freshly isolated mitochondria by MitoTracker Red/MitoTracker Green staining. MitoTracker Red/MitoTracker green ratio was calculated and presented relative to freshly isolated mitochondria. Error bars represent standard deviation of technical replicates (n=3). FIG. 5B. Mitochondria were frozen as in FIG. 5A, and their ATP content was determined after one month of storage at −80° C. The values were normalized to control samples from the same mitochondrial isolations frozen at −80° C., to compensate for possible protein determination errors and biological variability between isolations. Data are presented as relative to 2 K/min. Error bars represent standard deviation of technical replicates (n=3).

FIG. 6. In vivo coronary blood flow experiment

Freshly isolated mitochondria from the animal's own skeletal muscle tissue (“fresh autologous”) or cultured human cardiac fibroblasts (“fresh HCF”), as well as frozen mitochondria isolated from cultured HCF cells, frozen in a buffer supplemented with cryoprotectants (7 mM HEPES, pH 7.2, 0.7 mM EGTA, 210 mM trehalose, 1.2 M proline and 353 mM glucose) and stored for up to 1 week at −80° C., were injected into coronary artery of an anesthetized healthy pig, and coronary blood flow (CBF) was measured before and after the injection. N=1, 1000 μg of mitochondria for each condition were diluted in 5 mL of sucrose-based isolation buffer for each injection.

### DETAILED DESCRIPTION

The present invention relates to a cryopreservative composition and method suitable for the cryopreservation of isolated viable mitochondria. In one aspect, the cryopreservative composition of the invention further comprises isolated viable mitochondria, such as human mitochondria. According to the present invention the cryopreservative composition and the mitochondria comprised therein, are first frozen, then stored at low temperature, e.g., cryopreserved for weeks, months, or years, and, after thawing, are ready for use, for instance, in the treatment of mitochondrial or mitochondria-related diseases, ischemic or ischemic reperfusion related disorders/injuries, e.g., by facilitating the repairing process of damaged cells or tissues (Methods Mol Biol (2021), 2277: 15-37, Weissig V., Edeas M. Editors; J. Thorac. Cardiovasc. Surg. (2017), 154, 286-289, Autologous mitochondrial transplantation for dysfunction after ischemia-reperfusion injury, Emani, S. M., Del Nido, P. J., McCully J. D. et al.; Biochimica et Biophysica Acta (BBA)—Bioenergetics Vol. 1847, Issue 11, (November 2015), pp. 1387-1400, R. K. Lane et al.), infectious, inflammatory, or autoimmune diseases, for use in coronary artery interventions (WO 2017/124037, McCully et al.), or for diagnostics or research uses. The composition of the invention is also for use in in gene therapies. Examples of gene therapy for the treatment of cancer, infectious diseases, or autoimmune diseases, is the mitochondria transplantation into immune cells to produce mitochondria-enhanced immune effector cells, such as, but not limited to, chimeric antigen receptor (CAR) T-cell, CAR-NK cell, CAR-macrophage, neutrophil, tumor-infiltrating lymphocyte (TIL), gamma-delta T cell, in particular mitochondria-enhanced chimeric antigen receptor (CAR) T-cell or mitochondria-enhanced tumor-infiltrating lymphocyte (TIL) (WO2021203046A1, Schueller A. et al.).

The compositions and the methods of the present invention provide isolated mitochondria, e.g., isolated viable respiration-competent mitochondria, which can be successfully cryopreserved, stockpiled, transported, and then restored for later use. The cryopreserved composition of the present invention has a shelf-life of at least 3 months, such as of at least 6 months, e.g., 12 months or 24 months.

The cryopreserved isolated mitochondria, e.g., cryopreserved isolated viable mitochondria, comprised in the composition of the invention are ready-to-use and have advantages in terms of ease of application in research and clinical settings and in terms of efficacy. Indeed, the cryopreserved compositions of the invention as well as the isolated mitochondria, e.g., isolated viable mitochondria, comprised therein are readily available to be utilized in diverse biomedical techniques, whose objective is to improve or rescue the mitochondrial functions in cells and tissues, in particular in damaged cells and tissues.

### Compositions

### Embodiment A1: Cryopreservative Composition

In one aspect, the invention relates to a composition comprising:
(i) an aqueous buffer which has a pH from 5.5 to 8.5;
(ii) trehalose at a concentration of at least 150 mM; and;
(iii) a cryoprotecting agent selected from the group consisting of amino acid(s), sugar(s), polymer(s) or a combination thereof, wherein the amino acid has a concentration of (i.e., the total concentration of amino acid in the composition is) at least 150 mM, the sugar has a concentration of (i.e., the total concentration of sugar in the composition is) at least 150 mM, such as at least 160 mM, and the polymer has a concentration (in the composition) of 2.5% (w/v) to 30% (w/v), such as of 16% (w/v) to 30% (w/v). The composition preferably is a cryopreservative composition. The composition is preferably (suitable) for the cryopreservation of mitochondria as defined herein. Preferably, the mitochondria are viable and/or isolated mitochondria.

Exemplary cryoprotecting agents, which are comprised in the composition of the embodiment above either alone or in combination thereof are listed in Table 1a below:

TABLE 1a

| | |
|---|---|
| CRYO6 | 6.0M L-Proline |
| CRYO11 | 100% Polyethylene glycol 200 |
| CRYO12 | 100% Polyethylene glycol 400 |
| CRYO13 | 100% Polyethylene glycol monomethyl ether 550 |
| CRYO14 | 80% v/v Polyethylene glycol 600 |
| CRYO15 | 50% w/v Polyethylene glycol 1,000 |
| CRYO16 | 50% w/v Polyethylene glycol 3,350 |
| CRYO17 | 50% w/v Polyethylene glycol 4,000 |
| CRYO18 | 50% w/v Polyethylene glycol monomethyl ether 5,000 |
| CRYO19 | 50% w/v Polyethylene glycol 8,000 |
| CRYO20 | 50% w/v Polyethylene glycol 10,000 |
| CRYO21 | 50% w/v Polyvinylpyrrolidone K 15 |
| CRYO22 | 50% v/v Pentaerythritol propoxylate (5/4 PO/OH) |
| CRYO23 | 100% Polypropylene glycol P 400 |
| CRYO25 | 70% w/v D-(+)-Sucrose |
| CRYO26 | 70% w/v D-Sorbitol |
| CRYO27 | 30% w/v D-(+)-Maltose monohydrate |
| CRYO28 | 35% w/v meso-Erythritol |
| CRYO29 | 70% w/v Xylitol |
| CRYO30 | 15% w/v myo-Inositol |
| CRYO31 | 20% w/v D-(+)-Raffinose pentahydrate |
| CRYO32 | 50% w/v D-(+)-Trehalose dihydrate |
| CRYO33 | 70% w/v D-(+)-Glucose monohydrate |
| CRYO47 | 30% w/v Polyethylene glycol 3,350, 20% v/v Glycerol |

All the percentages weight/volume or volume/volume (% (w/v) or % (v/v)) as well as the molarities (M) of each cryoprotective agent of Table 1a above or of each cryoprotective agent of Table 1c and Table 1b below are provided for aqueous solutions or suspensions, i.e., for solution or suspension wherein the solvent is water.

The cryoprotecting agent is added to (i) the aqueous buffer comprised in the cryopreservative composition in such an amount as to obtain the desired concentration of the cryoprotectant, in the final total volume of the composition. For example, the cryoprotecting agent CRYO25, which is a solution of 70% w/v D-(+)-Sucrose, contains 700 g of sucrose/1 liter of water. Being the molecular weight of sucrose equal to 342.3 g/mol, the solution of 70% w/v D-(+)-Sucrose, contains sucrose at a concentration measured in molarity of about 2.0 M. This aqueous solution of 2.0 M sucrose has been added to the composition in in such an amount as to obtain, for example, a concentration of 150 mM in the final total volume of the composition. For example, the cryoprotecting agent CRYO33, which is a solution of 70% w/v D-(+)-Glucose monohydrate, contains 700 g of glucose monohydrate/1 liter of water. Being the molecular weight of glucose monohydrate equal to 198.17 g/mol, the solution of 70% w/v D-(+)-Glucose monohydrate, contains glucose at a concentration measured in molarity of about 3.5 M. This aqueous solution of 3.5 M glucose will be added to the composition in in such an amount as to obtain for example a concentration of 150 mM. For instance, a composition (e.g., suspension) comprising 150 mM of CRYO6, i.e., 150 mM of L-proline, was prepared by adding 10 μL of a 6 M proline solution (i.e., CRYO6) in a final volume of 400 μL of the composition containing 300 mM trehalose, 1 mM EGTA, 10 mM HEPES (pH 7.2).

In accordance with the above, exemplary cryoprotecting agents, which can be comprised in the composition above either alone or in combination thereof are given below:

TABLE 1c

| | |
|---|---|
| CRYO6 | L-Proline |
| CRYO11 | Polyethylene glycol 200 |
| CRYO12 | Polyethylene glycol 400 |
| CRYO13 | Polyethylene glycol monomethyl ether 550 |
| CRYO14 | Polyethylene glycol 600 |
| CRYO15 | Polyethylene glycol 1,000 |
| CRYO16 | Polyethylene glycol 3,350 |
| CRYO17 | Polyethylene glycol 4,000 |
| CRYO18 | Polyethylene glycol monomethyl ether 5,000 |
| CRYO19 | Polyethylene glycol 8,000 |
| CRYO20 | Polyethylene glycol 10,000 |
| CRYO21 | Polyvinylpyrrolidone K 15 |
| CRYO22 | Pentaerythritol propoxylate (5/4 PO/OH) |
| CRYO23 | Polypropylene glycol P 400 |
| CRYO25 | D-(+)-Sucrose |
| CRYO26 | D-Sorbitol |
| CRYO27 | D-(+)-Maltose monohydrate |
| CRYO28 | meso-Erythritol |
| CRYO29 | Xylitol |
| CRYO30 | myo-Inositol |
| CRYO31 | D-(+)-Raffinose pentahydrate |
| CRYO32 | D-(+)-Trehalose dihydrate |
| CRYO33 | D-(+)-Glucose monohydrate |
| CRYO47 | Polyethylene glycol 3,350, Glycerol |

In Table 1c above are provided example of commercially available cryoprotecting agents.

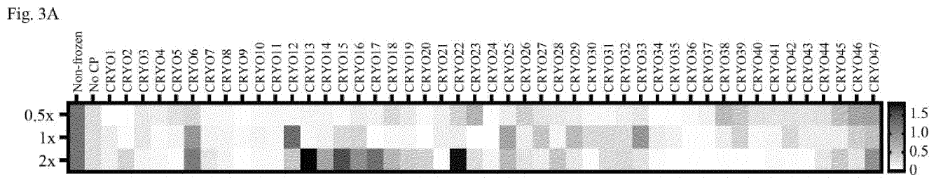

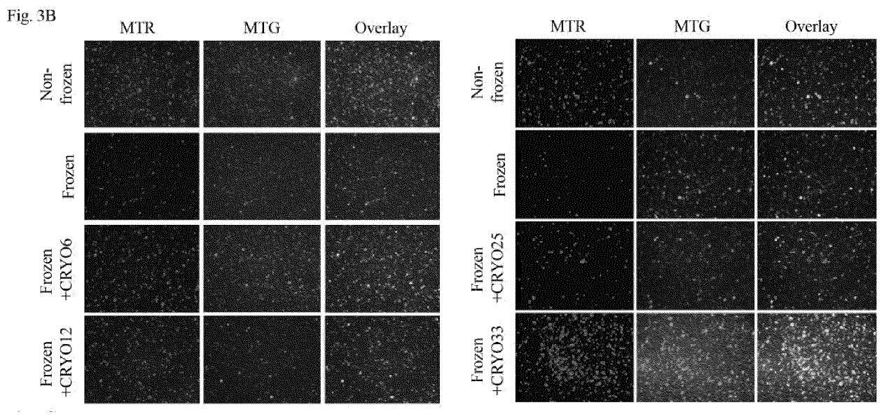

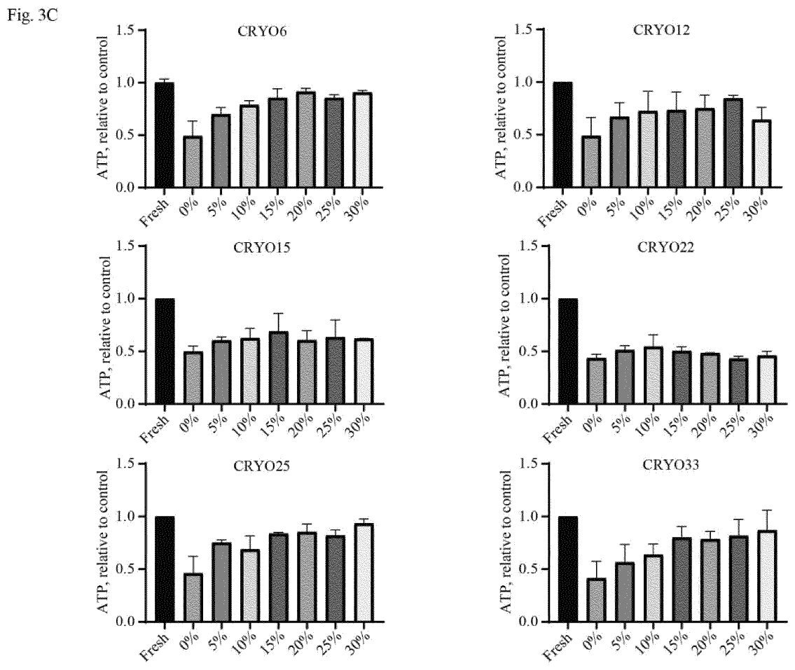

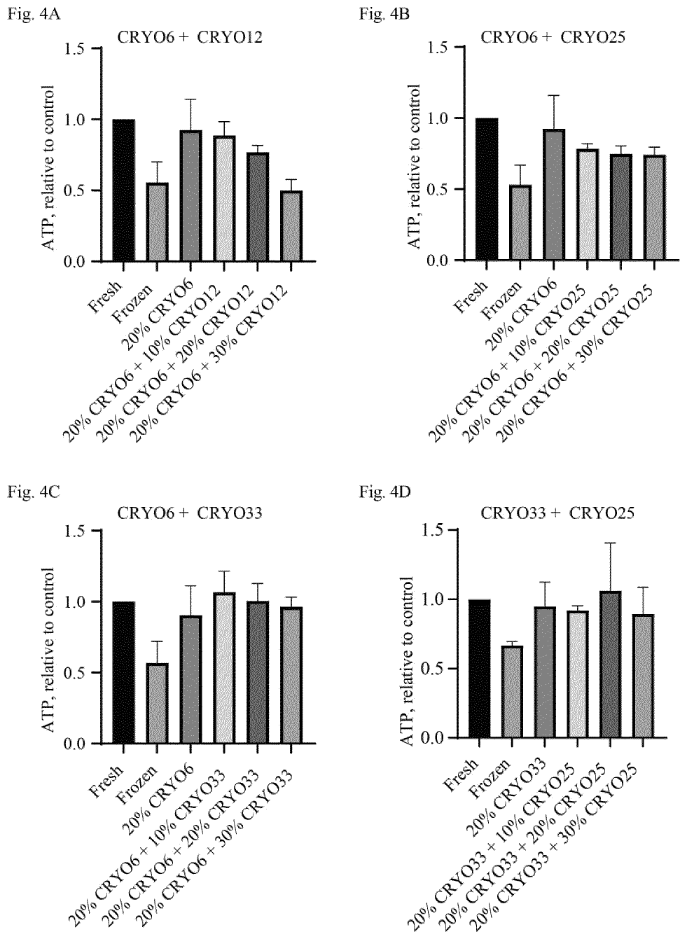

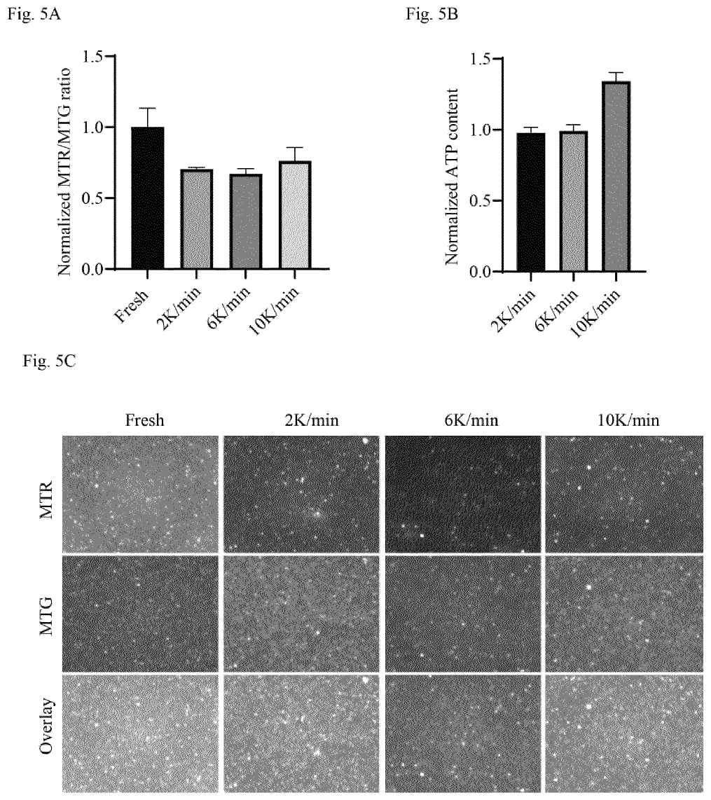

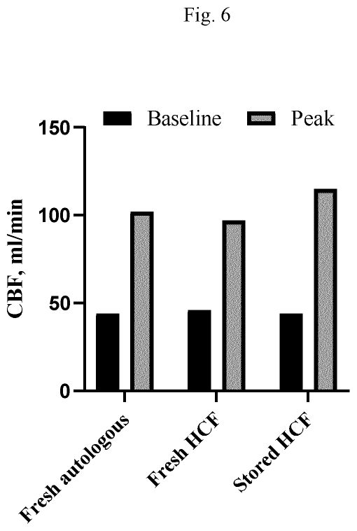

The cryoprotecting agents described in the Table 1c above have been used for the preparation of the composition by diluting the solution at 5%, 10% and 20% (v/v) (see FIG. 3A and FIG. 3B and CRYO1-CRYO47 of Table 1b), at 5%, 10% 15%, 20%, 25% and 30% (v/v) (see FIG. 3C for CRYO6, CRYO12, CRYO15, CRYO22, CRYO25, and CRYO33) and at 10%, 20%, and 30% (v/v) (FIGS. 4A-D for CRYO6+CRYO12, CRYO6+CRYO25, CYRO6+CRYO33 and CRYO33+CRYO25) in mitochondrial “trehalose-based” isolation buffer (1).

Preferred cryoprotectants are: CRYO13, CRYO14, CRYO15, CRYO16, CRYO17, CRYO18, CRYO22, CRYO23, CRYO25, CRYO38, CRYO46, and/or CRYO47. More preferably, the composition comprises a cryoprotectant selected from the group consisting of CRYO6, CRYO12, CRYO25 and/or CRYO33.

In one aspect, the aqueous buffer comprised in the composition of the above embodiment has a pH from 5.8 to 8.5, such as 6.0, 6.2, 6.4, 6.6, 6.8, 7.0 or 7.5. In certain embodiments the aqueous buffer has a pH from 6.5 to 8.5, such as 6.7, 6.9, 7.1, 7.3, 7.7 or 7.9. In certain preferred embodiments, the aqueous buffer has a pH from 6.8 to 8.2, such as 7.0, 7.2, 7.4, 7.6, 7.8 or 8.0, such as pH 7.2. The composition provided herein preferably is an aqueous composition. Preferably, the (aqueous) composition has the same pH as the aqueous buffer comprised therein. The definitions and explanations in relation to the pH provided herein for the aqueous buffer apply mutatis mutandis for the (aqueous) composition. Preferably, deionized and/or RNase/DNase-free water is used for preparing the aqueous composition and/or aqueous buffer. Further, in one aspect, the (sole) aqueous component of the (aqueous) composition is the herein described aqueous buffer. Amino acid(s), sugar(s) and/or polymer(s) may be (directly) dissolved in the aqueous buffer. Alternatively, amino acid(s), sugar(s) and/or polymer(s) may be first dissolved in a non-aqueous solution and subsequently added to the aqueous buffer or the aqueous composition

In one aspect, the aqueous buffer comprised in the composition of any one of the above embodiments comprises a buffering agent, preferably pH buffering agent. The buffering agent can be, selected from, but is not limited to, the group of agents comprising 2-[4-(2-hydroxyethyl)-piperazin-1-yl]-ethanesulfonic acid (HEPES), piperazine-N,N′-bis(2-ethanesulfonic acid) (PIPES), 4-morpholineethanesulfonic acid (MES), bis-(2-hydroxyethyl)amino-tris-(hydroxymethyl)-methane (Bis-Tris), 2-(N-cyclohexylamino)-ethanesulfonic acid (CHES), N,N-Bis-(2-hydroxyethyl)-glycine (Bicine), potassium phosphate, sodium cacodylate, tris-(hydroxymethyl)aminomethane hydrochloride) (Tris), 4-morpholinepropanesulfonic acid (MOPS), 1,3-bis-[tris-(hydroxymethyl)-methylamino]-propane (Bis-Tris propane), sodium acetate, or a combination thereof. In certain embodiments, the pH buffering agent has a concentration of 0.5 mM to 50 mt, such as, 1 mM to 40 mM, preferably of 2 mM to 35 mt, such as, 5 mM to 30 mM. In certain embodiments, the pH buffering agent has a concentration of 10 mM to 35 mM, e.g., 15 mM, 20 mM, 25 mM, or 30 mM. In certain preferred embodiments, the aqueous buffer comprises HEPES, e.g., 10 mM HEPES (adjusted with KOH to pH 7.2).

In another aspect, the composition of any one of the above embodiments, further comprises a calcium chelator. In certain embodiments the calcium chelator is ethylene glycol-bis(3-aminoethyl ether)-N,N,N′,N′-tetraacetic acid (EGTA), 2,2′,2″,2′″-(Ethane-1,2-diyldinitrilo)-tetraacetic acid (EDTA), 1,2-bis(o-aminophenoxy)ethane-N,N,N′,N′-tetraacetic acid (BAPTA), 1,2-bis(2-aminophenoxy)ethane-N,N,N′,N′-tetraacetic acid tetrakis-(acetoxymethyl ester) (BAPTA-AM) or a combination thereof. In certain preferred embodiments the calcium chelator is EDTA or EGTA. In certain embodiments, the calcium chelator has a concentration of 0.1 mM to 10 mM, such as, for example, of 0.5 mM to 5 mM, such as 1 mM or 1.5 mM. In one preferred embodiment, the calcium chelator is EGTA at a concentration of 0.5 mM to 2 mM, such as 1 mM. In another preferred embodiment, the calcium chelator is EDTA at a concentration of 0.5 mM to 2 mM, such as 1 mM.

In another aspect, the composition of any one of the above embodiments, further comprises an ionic component. In certain embodiments, the ionic component includes salts, acids, or bases that provide ions such as Mg²⁺, Na⁺, K⁺, Cl⁻, HCO₃⁻. or the like, or a combination thereof. In further embodiments the ionic components are, for example, suitable salts, which include, but are not limited to, MgCl₂. MgSO₄, KCl, KH₂PO₄, NaHCO₃, Na₂HPO₄, C₂H₂MgO₄ (magnesium formate), C₃H₃NaO₃ (sodium pyruvate), C₂H₃NaO₂ (sodium acetate), or the like, or a combination thereof. Alternatively, the ionic component is an organic anion derived from an organic acid. In certain embodiments the organic anion includes, but it is not limited to, citrate, pyruvate, malate, oxaloacetate, formate, glutamate, α-ketoglutarate, succinate, and acetate anions. In certain embodiments, the ionic components are selected from the group consisting of lithium acetate dihydrate, lithium chloride, lithium formate monohydrate, lithium nitrate, lithium sulfate monohydrate, sodium malonate pH 7.0, magnesium acetate tetrahydrate, sodium chloride, sodium formate, sodium nitrate, and sodium sulfate decahydrate. In certain embodiment the ionic component ranges from 0.01% (w/v) to 10% (w/v), such as, for example, from 0.1% (w/v) to 10% (w/v), such as 0.5% (w/v), 1.0% (w/v), 1.5% (w/v), 2.5% (w/v), 3% (w/v) or 5% (w/v). In certain embodiments the ionic components ranges from 0.5% (w/v) to 9% (w/v), from 1% (w/v) to 8% (w/v), from 2% (w/v) to 6% (w/v), such as 2.5% (w/v), 3% (w/v) or 5% (w/v). In further embodiments, the ionic component has a concentration (e.g., concentration expressed in molarity), which ranges, for example, from 0.01 mM to 200 mM, such as, for example from 0.1 mM to 180 mM, from 0.5 mM to 150 mM, from 1 mM to 120 mM, from 1 mM to 100 mM, such as 3 mM, 5 mM, 10 mM, 20 mM, 40 mM, 50 mM, 70 mM, or 90 mM. In certain preferred embodiments the ionic component has a concentration of 1 mM to 30 mM, such as 15 mM. In other preferred embodiments the ionic component has a concentration of 2 to 20 mM, such as, for example, 4 mM, 6 mM, 8 mM, 12 mM, 14 mM, 16 mM, or 18 mM.

In another aspect, the composition of any one of the above embodiments, further comprises albumin. In certain embodiment the albumin is bovine serum albumin (BSA), human serum albumin (HSA), or the like, or a combination thereof. In one embodiment the albumin is BSA. In another embodiment the albumin is HSA. In certain embodiments, the albumin has a concentration of 0.01% (w/v) to 10% (w/v), such as, 0.05% (w/v) to 5% (w/v), 0.1% (w/v) to 3% (w/v), 0.3% (w/v) to 2% (w/v), 0.5% (w/v) to 1.5% (w/v) or 0.8% (w/v) to 1% (w/v). In one preferred embodiment the albumin has a concentration of 0.1% (w/v), e.g., BSA 0.1% (w/v).

Serum albumin primarily modulates the osmotic or oncotic pressure. Additionally, it contributes to the saturation of the proteases, and the binding of fatty acids.

In another aspect, the composition of any one of the embodiments above comprises 20 mM Tris (adjusted to pH 7.4 with HCl), 2 mM EDTA and 10 mM MgCl₂. In another embodiment, the composition of any one of the embodiments above comprises 5 mM MOPS (adjusted to pH 7.25 with KOH), 10 mM BAPTA and 5 mM sodium pyruvate. In yet a further embodiment, the composition of any one of the embodiments above comprises 10 mM HEPES (adjusted to pH 7.2 with KOH) and 1 mM EGTA.

In another aspect, the composition of any one of the embodiments above includes less than a cryopreservative amount of permeable cryoprotectants or no permeable cryoprotectants. The permeable cryoprotectant is for instance selected from the group consisting of propylene glycol, ethylene glycol, glycerol and dimethyl sulfoxide (DMSO). In one particular embodiment, the composition of any one of the embodiments above includes less than a cryopreservative amount of dimethyl sulfoxide (DMSO) or no dimethyl sulfoxide (DMSO).

In one aspect, the composition of any one of the above embodiments comprises trehalose at a concentration of at least 160 mM, such as, for example, at least 170 mM, at least 180 mM, at least 190 mM, at least 200 mM, at least 210 mM, at least 220 mM, at least 230 mM or at least 240 mM. In one embodiment, the concentration of trehalose is at least 250 mt, such as 260 mM, at least 270 mM, at least 280 mM, at least 290 mM, or at least 300 mM.

In another aspect, the composition according to any one of the above embodiments comprises trehalose at a concentration of at least 150 mM and of no more than 1500 mM. In some particular embodiments, the composition according to any one of the above embodiments, comprises trehalose at a concentration of no more than 1400 mM, no more than 1300 mM, or no more than 1200 mM. In some particular embodiments, the composition according to any one of the above embodiments, comprises trehalose at a concentration of at least 150 mM and of no more than 1100 mM, such as for example no more than 1000 mM, no more than of 900 mM, no more than 800 mM, or no more than 700 mM. Preferably the composition according to any one of the above embodiments, comprises trehalose at a concentration of at least 150 mM and of no more than 650 mM, such as for example, no more than 640 mM, no more than 630 mM, no more than of 620 mM, or no more than 610 mM. In another preferred embodiment, the composition according to any one of the above embodiments, comprises trehalose at a concentration of at least 150 mM and of no more than 600 mM, such as, for example, no more than 590 mM, no more than of 580 mM, no more than of 570 mM, or no more than 560 mM. In a more preferred embodiment, the composition according to any one of the above embodiments, comprises trehalose at a concentration of at least 150 mM and of no more than 550 mM, such as, for example, no more than 540 mM, no more than of 530 mM, no more than of 520 mM, or no more than 510 mM. In another more preferred embodiment, the composition according to any one of the above embodiments, comprises trehalose at a concentration of at least 150 mM and of no more than 500 mM, such as, for example, no more than 490 mM, no more than of 480 mM, no more than of 470 mM, or no more than 460 mM. Even more preferably, the composition according to any one of the above embodiments comprises trehalose at a concentration of at least 150 mM and of no more than 450 mM. In some particularly preferred embodiments, the composition according to any one of the above embodiments, comprises trehalose at a concentration of 150 mM to 450 mM, such as, 160 mM to 440 mM, 170 mM to 430 mM, 180 mM to 420 mM, or 190 mM to 410 mM. In some other particularly preferred embodiments, the composition according to any one of the above embodiments comprises trehalose at a concentration of 200 mM to 400 mM, such as, 220 mM to 380 mM, such as 240 mM, 260 mM, 280 mM, 320 mM, 340 mM, Or 360 mM. In some other particularly even more preferred embodiments, trehalose has a concentration of 250 mM to 350 mM, such as 300 mM.

In one aspect, the sugar comprised in the composition of any one of the embodiments above, e.g., the sugar of (iii) the cryoprotecting agent contained in the composition, is selected from the group consisting of any suitable mono-, di-, tri-, oligo-, polysaccharide, or a combination thereof. In one preferred aspect, the sugar comprised in the composition of any one of the embodiments above, e.g., the sugar of (iii) the cryoprotecting agent contained in the composition, is selected from the group consisting of any suitable mono-, di-, or trisaccharide, or a combination thereof. In one specific embodiment, sugar is all sugars other than trehalose. In certain embodiments sugar is a sugar derivative, such as a sugar alcohol. Exemplary sugar alcohols to be used herein are sorbitol, erythritol xylitol (e.g., D-sorbitol, meso-erythritol and/or xylitol, specifically 70% w/v D-sorbitol, 35% w/v meso-erythritol and/or 70% w/v xylitol), inositol, mannitol, arabitol, and/or ribitol. In some other embodiment, sugar is a modified sugar.

Preferably, the term “sugar” as used herein refers to sugar in the narrower sense, i.e., mono-, di-, tri-, oligo-, polysaccharide, more preferably mono- and/or disaccharides, as further defined and explained herein. In one particular embodiment, the sugar is maltose, lactose, fructose, sucrose, glucose, dextran, melezitose, raffinose, nigerotriose, maltotriose, maltotriulose, kestose, cellobiose, chitobiose, lactulose, or a combination thereof. In one embodiment, the sugar is preferably sucrose, glucose, or a combination thereof. In one embodiment, the sugar of (iii) the cryoprotecting agent is not trehalose. In one aspect, the sugar comprised in the composition according to any one of the embodiments above has a concentration of at least 150 mM. In a further aspect, the sugar comprised in the composition according to any one of the embodiments above has a concentration of at least 160 mM. In a further aspect, the sugar comprised in the composition according to any one of the embodiments above has a concentration of at least 170 mM, such as of at least 180 mM, at least 190 mM, at least 200 mM, at least 210 mM, at least 220 mM, at least 230 mM, or at least 240 mM. In further embodiments, the sugar has a concentration of at least 250 mM, such as at least 260 mM, at least 270 mM, at least 280 mM or at least 290 mM. In a further embodiment, the sugar has a concentration of at least 300 mM, such as, for example, at least 310 mM, at least 320 mM, at least 330 mM, at least 340 mM, at least 350 mM, at least 360 mM, at least 370 mM, at least 380 mM, at least 390 mM, at least 400 mM, at least 410 mM, at least 420 mM, at least 430 mM, at least 440 mM or least 450 mM. In certain preferred embodiments, the sugar has a concentration of 300 mM to 800 mM, such as 320 mM, 350 mM, 400 mM, 410 mM, 420 mM, 430 mM, 440 mM, 450 mM, 460 mM, 470 mM, 480 mM, 490 mM, 500 mM, 510 mM, 520 mM, 530 mM, 540 mM, 550 mM, 560 mM, 570 mM, 580 mM, 590 mM, 600 mM, 650 mM, 700 mM, or 750 mM. The sugar has a maximum concentration of (e.g., the total amount of sugar is) no more than 2000 mM such as, for example, no more than 1900 mM, or no more than 1800 mM. In one embodiment, the sugar has a maximum concentration of no more than 1700 mM, such as no more than 1600 mM, no more than 1500 mM, no more than 1400, no more than 1300 mM or no more than 1200 mM. In some preferred embodiments, the maximum concentration of sugar is no more than 1500 mM, such as, no more than 1300 mM, no more than 1000 mM, no more than 950 mM, no more than 900 mM, no more than 850 mM, no more than 800 mM, no more than 750 mM, or no more than 700 mM. The sugar is provided at a concentration within a range having endpoints defined by any minimum concentration listed above and any maximum concentration listed above that is greater than the minimum concentration. When more than one sugar is present in the composition of any one of the embodiments above, the concentration of the sugar reflects the total concentration of all sugar in the composition. In other preferred embodiments the sugar has a concentration of 250 mM to 650 mM, such as, 280 mM to 600 mM, such as, for example 300 mM, 400 mM, or 500 mM. Thus, in some embodiments, the sugar is present at a concentration of 150 mM to 1600 mM, such as of 200 mM to 1500 mM, 250 mM to 1200 mM, or of 300 mM to 1100 mM. In some embodiments the sugar has a concentration of 250 mM to 650 mM, such as, 280 mM to 600 mM, such as, for example 300 mM, 400 mM, or 500 mM. In certain embodiments, the sugar comprised in the composition of any one of embodiments above, is sucrose at a concentration of 150 mM to 700 mM, such as of 160 mM to 675 mM, such as of 200 mM to 650 mM, such as of 250 mM to 620 mM, such as of 300 mM to 600 mM, such as 400 mM or 500 mM. In other embodiments the composition includes glucose at a concentration of 300 mM to 1200 mM, such as, for example, of 450 mM to 1100, such as of 500 mM to 1000 mM, such as of 700 mM or 800 mM. In some particular embodiments, the composition of any one of the embodiments above comprises more than one sugar, such as, for example, fructose together with glucose or sucrose, or a combination of fructose, glucose, and sucrose. In some preferred embodiments, the composition of any one of the embodiments above comprises sucrose and glucose at a concentration (e.g., total concentration is) of at least 150 mM and no more than 2000 mM. In one preferred embodiment, the sugar is sucrose, glucose, or a combination thereof, and sucrose and glucose have a total concentration of at least 160 mM, such as 200 mM, 300 mM, 400 mM, 500 mM, 600 mM, 700 mM, 800 mM, 900 mM, 1000 mM, or 1500 mM. In another preferred embodiment, sucrose and glucose have a total concentration of 500 mM to 1800 mM, more preferably of 600 mM to 1700 mM, such as, 800 mM to 1450 mM, such as 900 mM, 1120 mM, or 1320 mM. In one aspect, the sugar(s) comprised in a composition, which contains no amino acid(s), preferably has a total concentration of at least 160 mM, such as of at least 180 mM or at least 200 mM.

The sugar is provided at a concentration within a range having endpoints defined by any minimum concentration listed above and any maximum concentration listed above that is greater than the minimum concentration. When more than one sugar is present in the composition of any one of the embodiments above, the concentration of the sugar reflects the total concentration of all sugar in the composition.

In one aspect, the amino acid comprised in the composition of any one of the embodiments above, e.g., the amino acid of (iii) the cryoprotecting agent contained in the composition, is any suitable amino acid, amino acid derivative, oligopeptide, peptide, or a combination thereof. For example, in one particular embodiment, the amino acid component is isoleucine (e.g., L-isoleucine), proline (e.g., L-proline), valine (e.g., L-valine), alanine (e.g., L-alanine), glycine, asparagine (e.g., L-asparagine), aspartic acid (e.g., L-aspartic acid), glutamic acid (e.g., L-glutamic acid), serine (e.g., L-serine), histidine (e.g., L-histidine), cysteine (e.g., L-cysteine), tryptophan (e.g., L-tryptophan), tyrosine (e.g., L-tyrosine), arginine (e.g., L-arginine), glutamine (e.g., L-glutamine), lysin, threonine, selenocysteine, methionine, phenylalanine, creatine (e.g., L-creatine), taurine (e.g., L-taurine), betaine, ectoine, dimethylglycine, ethylmethylglycine, glutathione, an RGD peptide (i.e., arginyl-glycyl-aspartic acid peptide), or a combination thereof. In certain preferred embodiments, the amino acid is proline (e.g., L-proline), glycine, or cysteine (e.g., L-cysteine). In other preferred embodiments, the amino acid is proline (e.g., L-proline), proline derivatives, such as, methylproline, benzylproline, hydroxyproline, aminoproline, dehydroproline, aziridinecarboxylic acid, azetidinecarboxylic acid, pipecolic acid (e.g., homoproline), oxaproline, thiaproline, or a combination thereof. In an even more preferred embodiment, the amino acid is proline (e.g., L-proline). The amino acid comprised in the composition of any one of the embodiments above has a concentration of at least 160 mM, such as, for example, at least 180 mM, at least 200 mM, at least 250 mM, at least 300 mM, at least 350 mM, at least 400 mM, at least 450 mM, at least 500 mM, at least 550 mM, at least 600 mM, at least 650 mM, at least 700 mM, at least 750 mM, at least 800 mM, at least 850 mM or at least 900 mM. Preferably the amino acid has a concentration of at least 1000 mM, such as, at least 1100 mM, at least 1200 mM, at least 1300 mM, at least 1400 mM, at least 1500 mM, at least 1600 mM, at least 1700 mM, at least 1800 mM, at least 1900 mM, or at least 2000 mM. The amino acid component may be provided at a maximum concentration of no more than 3000 mM such as, for example, no more 2900 mM, no more than 2850 mM, no more than 2800 mM, no more than 2750 mM, no more than 2700 mM, no more than 2650 mM, no more than 2600 mM, no more than 2550 mM, no more than 2500 mM, no more than 2450 mM, no more than 2400 mM, no more than 2350 mM, no more than 2300 mM, no more than 2250 mM, no more than 2200 mM, no more than 2150 mM, no more than 2100 mM, or no more than 2050 mM. The amino acid is provided at a concentration within a range having endpoints defined by any minimum concentration listed above and any maximum concentration listed above that is greater than the minimum concentration. When more than one amino acid is present in the composition, the concentration of the amino acid reflects the total concentration of all amino acid in the composition. Thus, in some embodiments, the amino acid is present at a concentration of 500 mM to 3000 mM, such as 800 mM to 2800 mM, or 1000 mM to 2500 mM. For example, in one preferred embodiment the amino acid is proline at a concentration of 600 mM. In other embodiments the amino acid is proline at a concentration of 650 mM to 2000 mM, such as, of 900 mM to 1900 mM, such as 1500 mM or 1800 mM. In one preferred embodiment the amino acid is proline at a concentration of 1000 mM to 1700 mM, such as 1500 mM. In another preferred embodiment, the amino acid is proline at a concentration of 1200 mM. In some other preferred embodiments, the amino acid is a proline derivative such as, but not limited to, methylproline, benzylproline, hydroxyproline, aminoproline, dehydroproline, aziridinecarboxylic acid, azetidinecarboxylic acid, pipecolic acid (e.g., homoproline), oxaproline, thiaproline, at a concentration of 150 mM to 3000 mM, such as, for example, of 200 mM to 2800 mM, or 300 mM to 2500 mM. In one preferred embodiment, the proline derivative is at a concentration of 500 mM to 2000 mM, such as, for example, of 600 mM to 1800 mM, such as, for example 800 mM, 1000 mM, 1300 mM, or 1500 mM.

In one aspect, the polymer comprised in the compositions of any one of the embodiments above, e.g., the polymer of (iii) the cryoprotecting agent of the composition, is a polymer, which has one or more of the following properties: it is biocompatible, hydrophilic, or amphiphilic. For example, the polymer component includes poloxamer (e.g., poloxamer 142, poloxamer 188, poloxamer 331, or poloxamer 407), alginate, polyethylene glycol (PEG), e.g., PEG400 or PEG1000, polyglutamic acid, polyvinyl alcohol, polyvinyl pyrrolidone, or a combination thereof. In certain preferred embodiments, the polymer is alginate or polyethylene glycol (PEG). In more preferred embodiments, the polymer is polyethylene glycol (PEG). The polymer comprised in the composition of any one of the embodiments above has a concentration of at least 2.5% (w/v), at least 3% (w/v), at least 4% (w/v), at least 5% (w/v), at least 6% (w/v), at least 7% (w/v), at least 8% (w/v) at least 9% (w/v), at least 10% (w/v), at least 11% (w/v), at least 12% (w/v), at least 13% (w/v), at least 14% (w/v) or at least 15% (w/v). The polymer comprised in the composition of any one of the embodiments above has a concentration of at least 16% (w/v), such as of at least 18% (w/v). The polymer is provided at a maximum concentration of no more than 30% (w/v), no more than 25% (w/v), no more than 22% (w/v), no more than 21% (w/v), no more than 20% (w/v), or no more than 18% (w/v). The polymer is provided at a concentration within a range having endpoints defined by any minimum concentration listed above and any maximum concentration listed above that is greater than the minimum concentration. When more than one polymer is present in the composition, the concentration of the polymer reflects the total concentration of all polymers in the composition. Thus, in some embodiments, the polymer component is present at a concentration of 2.5% (w/v) to 30% (w/v), 2.5% (w/v) to 25% (w/v), 5% (w/v) to 25% (w/v), 10% (w/v) to 25% (w/v), 10% (w/v) to 20% (w/v), or 12% (w/v) to 18% (w/v). For example, in certain embodiments the polymer has a concentration of 8% (w/v) to 18% (w/v). In further particular embodiments the polymer has a concentration of 14% (w/v) to 16% (w/v). For example, in a preferred embodiment the polymer is polyethylene glycol (PEG) at a concentration of 2.5% (w/v) to 30% (w/v), more preferably at a concentration of 5% (w/v) to 25% (w/v), such as 10% (w/v), 15% (w/v) or 20% (w/v). In another preferred embodiment the polymer is polyethylene glycol (PEG) at a concentration of 16% (w/v) to 30% (w/v), more preferably at a concentration of 18% (w/v) to 25% (w/v), such as 20% (w/v), 21% (w/v), 23% (w/v), or 24% (w/v).

In one aspect, the polymer(s) comprised in the composition is/are the only cryoprotecting agent(s) and has/have a concentration of least 16% (w/v), such as of at least 20%/w/v) or at least 25% (w/v).

In a preferred embodiment the polymer contained in the composition comprising no amino acid(s) and no sugar(s), except trehalose, is polyethylene glycol (PEG) at a concentration of 16% (w/v) to 30% (w/v), such as at a concentration of 18% (w/v) to 28% (w/v), such as 20% (w/v) to 25% (w/v).

In one aspect, the composition of any one of the embodiments above comprises:
(i) an aqueous buffer having a pH from 5.5 to 8.5, such as a pH of 6.5 to 8.0, e.g., a pH of 7.2
(ii) trehalose at a concentration of at least of 150 mM; and
(iii) a cryoprotecting agent selected from proline, sucrose, glucose, polyethylene glycol (PEG) or a combination thereof, wherein the proline has a concentration of at least 150 mM, such as of at least 500 mM, preferably of at least 1000 mM, e.g., 1200 mM, sucrose and glucose have a total concentration of at least 150 mM, such as of at least 200 mM, e.g., 300 mM or 500 mM, and the polyethylene glycol (PEG) has a concentration of 2.5% (w/v) to 30% (w/v), such as of 5% (w/v) to 25% (w/v), e.g., 10% (w/v), 15% (w/v) or 20% (w/v).

In another aspect, the composition of any one of the above embodiments comprises an aqueous buffer as described in any one of the embodiments above.

In another aspect, the composition of any one of the above embodiments, further comprises a calcium chelating agent as described in any one of the embodiments above.

In another aspect, the composition of any one of the above embodiments, further comprises ionic component as described in any one of the embodiments above.

In another aspect, the composition of any one of the above embodiments, further comprises albumin as described in any one of the embodiments above.

In another aspect, the composition of any one of the embodiments above comprises 20 mM Tris (adjusted to pH 7.4 with HCl), 2 mM EDTA and 10 mM MgCl₂. In another embodiment, the composition of any one of the embodiments above comprises 5 mM MOPS (adjusted to pH 7.25 with KOH), 10 mM BAPTA and 5 mM sodium pyruvate. In yet a further embodiment, the composition of any one of the embodiments above comprises 10 mM HEPES (adjusted to pH 7.2 with KOH) and 1 mM EGTA.

In another aspect, the composition of any one of the embodiments above includes less than a cryopreservative amount of permeable cryoprotectants or no permeable cryoprotectants. The permeable cryoprotectant is for instance selected from the group consisting of propylene glycol, ethylene glycol, glycerol and dimethyl sulfoxide (DMSO). In one particular aspect, the composition of any one of the embodiments above includes less than a cryopreservative amount of dimethyl sulfoxide (DMSO) or no dimethyl sulfoxide (DMSO).

In one aspect, the composition according to any one of the above embodiments comprises trehalose at a concentration as defined in any one of the embodiments above.

In one aspect, the composition according to any one of the embodiments above comprises sucrose and glucose at a total concentration of at least 150 mM and no more than 2000 mM, such as of 150 mM to 1800 mM, such as 200 mM to 1750 mM. In certain embodiments the composition comprises sucrose and glucose at a total concentration of at least 250 mM, such as 250 mM to 1650 mM. In a particular embodiment, sucrose and glucose have a total concentration of 500 mM to 1500 mM, such as 600 mM to 1400 mM, such as, 800 mM to 1320 mM, such as, for example of 900 mM, or 1120 mM.

In one aspect, proline comprised in the composition of any one of the embodiments above has a concentration of at least 200 mM, such as 300 mM or 400 mM. In certain embodiments, proline has a concentration of at least 500 mM, such as, 600 mM, 700 mM, 800 mM, or 900 mM. In one more preferred embodiment, proline has a concentration of 600 mM. In other preferred embodiments the proline comprised in any one of the compositions of the invention above has a concentration of at least 1000 mM. In one more preferred embodiment, proline has a concentration of 1200 mM. In other preferred embodiments, proline has a concentration of at least 1300 mM, such as of at least 1500 mM, 1600 mM, 1800 mM, 1900 mM, 2000 mM, or 2200 mM.

In another aspect, the cryoprotecting agent of the composition according to any one of the above embodiments, is preferably a combination of proline and sucrose. In one particular embodiment the cryoprotecting agent is a combination of proline and sucrose, wherein preferably proline is at a concentration of at least 500 mM, such as 600 mM, and sucrose is at a concentration of 150 mM to 650 mM, such as, for example of 200 mM, 300 mM, 400 mM, 500 mM, or 600 mM. In one particular embodiment the cryoprotecting agent is a combination of proline and sucrose, wherein preferably proline is at a concentration of at least 1000 mM, such as 1200 mM, and sucrose is at a concentration of 150 mM to 650 mM, such as, for example of 200 mM, 300 mM, 400 mM, 500 mM, or 600 mM.

In a preferred embodiment the cryoprotecting agent of the composition according to any one of the embodiments above consists of proline and sucrose, wherein the proline is at a concentration of 10% volume of proline (i.e., 600 mM of proline) over the final total volume of the suspension (i.e., % (v_{CRYO}/vₜₒₜ)) and sucrose is at a concentration of 5%, 10%, 15%, 20%, 25%, or 30% volume of sucrose over the final total volume of the suspension (i.e., % (v_{CRYO}/vₜₒₜ)), i.e. sucrose is at a concentration respectively of about 100 mM, 200 mM, 300 mM, 400 mM, 500 mM, or 600 mM. In another preferred embodiment the (iii) cryoprotecting agent of the composition according to any one of the embodiments above consists of proline and sucrose, wherein the proline is at a concentration of 20% volume of proline (i.e., 1200 mM of proline) over the final total volume of the suspension (i.e., % (v_{CRYO}/vₜₒₜ)) and sucrose is at a concentration of about 100 mM, 200 mM, 300 mM, 400 mM, 500 mM, or 600 mM.

In another aspect, the cryoprotecting agent of the composition according to any one of the above embodiments, is preferably a combination of proline and glucose In one particularly preferred embodiment the cryoprotecting agent is a combination of proline and glucose, wherein proline is at a concentration of at least 500 mM, such as 600 mM, and glucose is at a concentration of 180 mM to 1500 mM, preferably of 350 to 1200 mM, such as, for example of 450 mM, 550 mM, 700 mM, 850 mM, 900 mM, 1000 mM, or 1100 mM. In another particularly preferred embodiment the cryoprotecting agent is a combination of proline and glucose, wherein proline is at a concentration of at least 1000 mM, such as 1200 mM, and glucose is at a concentration of 180 mM to 1500 mM, preferably of 350 to 1200 mM, such as, for example of 450 mM, 550 mM, 700 mM, 850 mM, 900 mM, 1000 mM, or 1100 mM.

In a preferred embodiment the cryoprotecting agent of the composition according to any one of the embodiments above consists of proline and glucose, wherein the proline is at a concentration of 10% volume of proline (i.e., 600 mM of proline) over the final total volume of the suspension (i.e., % (v_{CRYO}/vₜₒₜ)) and glucose is at a concentration of 5%, 10%, 15%, 20%, 25%, or 30% volume of glucose over the final total volume of the suspension (i.e., % (v_{CRYO}/vₜₒₜ)), i.e., glucose is respectively at a concentration of about 175 mM, 350 mM, 525 mM, 700 mM, 875 mM, or 1050 mM. In another preferred embodiment the (iii) cryoprotecting agent of the composition according to any one of the embodiments above consists of proline and glucose, wherein the proline is at a concentration of 20% volume of proline (i.e., 1200 mM of proline) over the final total volume of the suspension (i.e., % (v_{CRYO}/vₜₒₜ)) and glucose is at a concentration of about 175 mM, 350 mM, 525 mM, 700 mM, 875 mM, or 1050 mM.

Yet, in another aspect, the cryoprotecting agent of the composition according to any one of the to any one of the above embodiments, is a combination of proline and polyethylene glycol (PEG). In one particular embodiment the cryoprotecting agent is: proline at a concentration of at least 500 mM, preferably, of at least 1000 mM, e.g., 1200 mM, combined together with polyethylene glycol (PEG) at a concentration of 2.5% (w/v) to 30% (w/v), preferably of 5% (w/v) to 25% (w/v), such as, for example of 10% (w/v), 15% (w/v) or 20% (w/v).

In one aspect of the invention, the cryoprotecting agent comprised in the composition of any one of the embodiments above, is sucrose, glucose, or a combination thereof, at a concentration of (i.e., the total concentration of sucrose and glucose is) at least 200 mM, such as, of at least 300 mM, such as 400 mM or 500 mM. In certain embodiments sucrose and glucose have a concentration of (i.e., the total concentration of sucrose and glucose is) at least 600 mM, such as 850 mM to 1550 mM, such as, 900 mM to 1400, such as 1000 mM or 1350 mM. In one embodiment, the cryoprotecting agent is sucrose alone at a concentration of at least 250 mM and no more than 1200 mM, such as of at least 300 mM and no more than 650 mM, e.g., 400 mM and 500 mM. In another preferred embodiment, the cryoprotecting agent is glucose alone at a concentration of at least 450 mM and no more than 1550 mM, such as of at least 500 mM and no more than 1400 mM, such as of at least 700 mM to 1200 mM, e.g., 850 mM or 950 mM. In another preferred embodiment, the cryoprotecting agent comprised in the composition of any one of the embodiments above is a combination of sucrose and glucose at a total concentration of 550 mM to 1850 mM, such as, of 800 mM to 1700 mM, e.g., at a concentration of 900 mM, 1150 mM, 1400 mM, or 1500 mM. For example, the combination of sucrose and glucose comprises sucrose at a concentration between 200 mM and 650 mM, e.g., 400 mM or 500 mM and glucose at a concentration between 350 mM and 1350 mM, e.g., 530 mM, 880 mM, or 1100 mM.

In one aspect of the invention, the cryoprotecting agent comprised in the composition according to any one of the embodiments above, is polyethylene glycol (PEG), e.g., PEG400 or PEG1000, at a concentration of between 2.5% (w/v) to 25% (w/v), such as 5% (w/v) to 25% (w/v), such as, for example, at a concentration of 10% (w/v), 15% (w/v), or 20% (w/v).

In another aspect of the invention, the only cryoprotecting agent(s) comprised in the composition according to any one of the embodiments above, is the polymer(s), such as polyethylene glycol (PEG), e.g., PEG400 or PEG1000, at a concentration of preferably between 16% (w/v) to 30% (w/v), such as 18% (w/v) to 28% (w/v), e.g., 20% (w/v), 22% (w/v), 24% (w/v), 25% (w/v), or 26% (w/v).

In one aspect of the invention, the cryoprotecting agent comprised in the cryopreservative composition according to any one of the embodiments above is proline at a concentration of at least 150 mM. In certain embodiments, the proline has a concentration of at least 300 mM, such as of at least 500 mM, such as 600 mM. In one preferred embodiment, proline has a concentration of at least 600 mM, such as, for example, of 700 mM, 800 mM, or 900 mM. In another preferred embodiment, proline has a concentration of at least 1000 mM, such as, for example, 1200 mM. In other particular embodiments, proline has a concentration of at least 1300 mM, such as, for example, of 1500 mM or 1800 mM. In certain preferred embodiments, proline has a concentration of at least 600 mM and no more than 3000 mM, such as, of at least 1000 mM and no more than 2500 mM, such as, for example, of 1300 mM to 2200 mM, e.g., 1500 mM, 1600 mM, 1700 mM, 1800 mM, 1900 mM or 2000 mM.

In one aspect of the invention, the cryopreservative composition, comprises:
(i) a aqueous buffer having a pH from 6.0 to 8.0, such as pH 7.2;
(ii) trehalose at a concentration of 300 mM;
(iii) a cryoprotecting agent comprising proline in combination with sucrose or glucose,
wherein
   the proline has a concentration of 1200 mM;
   the sucrose has a concentration of 200 mM to 1700 mM, such as, for example, of 300 mM to 1500 mM, such as of 400 mM to 1300 mM, e.g., 500 mM or 600 mM; and
   the glucose has a concentration of 300 mM to 1700 mM, such as, for example, of 450 mM to 1500 mM, such as, for example, of 600 mM to 1300 mM, e.g., 700 mM or 800 mM.

In one aspect of the invention, the cryopreservative composition, comprises:
(i) an aqueous buffer having a pH of 6.0 to 8.0, such as 7.2;
(ii) trehalose at a concentration of 300 mM;
(iii) a cryoprotecting agent comprising proline in combination with the polyethylene glycol (PEG),
wherein
   the proline has a concentration of 1200 mM;
   the polyethylene glycol (PEG) has a concentration between 5% (w/v) to 20% (w/v), such as 10% (w/v) or 15% (w/v).

In one aspect of the invention, the cryopreservative composition, comprises:
(i) a aqueous buffer having a pH from 6.0 to 8.0, such as pH 7.2;
(ii) trehalose at a concentration of 300 mM;
(iii) a cryoprotecting agent comprising proline in combination with sucrose or glucose,
wherein
   the proline has a concentration of 600 mM;
   the sucrose has a concentration of 200 mM to 1700 mM, such as, for example, of 300 mM to 1500 mM, such as of 400 mM to 1300 mM, e.g., 500 mM or 600 mM; and
   the glucose has a concentration of 300 mM to 1700 mM, such as, for example, of 450 mM to 1500 mM, such as, for example, of 600 mM to 1300 mM, e.g., 700 mM or 800 mM.

In one aspect of the invention, the cryopreservative composition, comprises:
(i) an aqueous buffer having a pH of 6.0 to 8.0, such as 7.2;
(ii) trehalose at a concentration of 300 mM;
(iii) a cryoprotecting agent comprising proline in combination with the polyethylene glycol (PEG),
wherein
   the proline has a concentration of 600 mM;
   the polyethylene glycol (PEG) has a concentration between 5% (w/v) to 20% (w/v), such as 10% (w/v) or 15% (w/v).

In another aspect of the invention, the composition of any one of the above embodiments comprises HEPES, e.g., 10 mM HEPES (adjusted at a pH between 6.5 and 7.5, such as pH 7.2, with KOH).

In another embodiment the composition of any one of the above embodiments comprises a calcium chelator, such as, EGTA or M EDTA, e.g., 1 mM EGTA or 1 mM EDTA.

In another embodiment, the composition of any one of the above embodiments further comprises an ionic component, such as, for example, Mg²⁺, Na⁺, K⁺, Cl⁻, HCO₃⁻, HPO₄²⁻, C₃H₃O₃⁻ (pyruvate anion), C₂H₂O₄²⁻ (formate anion), C₂H₃O₂⁻ (acetate anion), or a combination thereof.

In another aspect, the compositions of any one of the embodiments above further comprises albumin, such as for example, BSA or HAS.

### Embodiment A2: Cryopreservative Composition Comprising Isolated Viable Mitochondria

In another aspect of the invention, the compositions of any one of the embodiments above further comprises isolated mitochondria, e.g., isolated viable mitochondria. The composition is, for example, obtained by addition of isolated mitochondria, e.g., isolated viable mitochondria, to the composition of any one of the embodiments above. In a preferred embodiment the isolated mitochondria are mammalian mitochondria, e.g., isolated viable mammalian mitochondria. In an even more preferred embodiment, the isolated mitochondria are human mitochondria, e.g., isolated viable human mitochondria.

In another aspect, the composition of the embodiment above comprises mitochondria, such as human mitochondria, e.g., isolated viable human mitochondria, whose sources are organs, tissues, blood, e.g., cells circulating in the blood, or cells, e.g., culture cells. Exemplary cells include, but are not limited to, placenta cells, muscle tissue cells, skin fibroblasts, cardiac fibroblasts, cardiomyocytes, cultured cells, HeLa cells, prostate cancer cells, cancer cell lines, such as, for instance, HEPG2, A549, MCF7, or RKO, yeast, among others, and any mixture thereof. Exemplary tissues include, but are not limited to, skeletal muscle, liver, heart, brain, kidney, placenta, lung, prostate, and adipose tissue. The sources of cells and tissues are yeasts or animals, preferably mammals, more preferably humans.

In another aspect, the composition of any one of the above embodiments comprises isolated mitochondria, e.g., isolated viable mitochondria, at a concentration of at least 0.02 μg/μL, such as for example, at least 0.05 μg/μL, at least 0.1 μg/μL, at least 0.2 μg/μL, at least 0.5 μg/μL, at least 0.75 μg/μL, at least 1 μg/μL, at least 1.5 μg/μL, or at least 2 μg/μL. In one embodiment, the isolated mitochondria, e.g., isolated viable mitochondria, are at a concentration of at least 5 μg/μL, such as of at least 10 μg/μL. In a further embodiment, the isolated mitochondria, e.g., isolated viable mitochondria, comprised in the composition of any one of the embodiments above are at a concentration of at least 20 μg/μL, such as 25 μg/μL, 30 μg/μL, 35 μg/μL, 40 μg/μL, 45 μg/μL, or 50 μg/μL. The isolated mitochondria, e.g., isolated viable mitochondria, are provided at a maximum concentration of no more than 100 μg/μL, such as, for example, no more than 80 μg/μL, no more than 70 μg/μL, or no more than 60 μg/μL. In one embodiment, the isolated mitochondria, e.g., isolated viable mitochondria, are comprised in the composition at a concentration of 0.02 μg/μL to 10 μg/μL, such as, for example, 0.05 μg/μL to 8 μg/μL, such as, for example, at a concentration of 0.1 μg/μL to 5 μg/μL. In a further embodiment, the isolated mitochondria, e.g., isolated viable mitochondria, are comprised in the composition at a concentration of 0.1 μg/μL to 1 μg/μL, such as, for example, at a concentration of 0.2 μg/μL to 0.75 μg/μL.

In one aspect, the composition of any one of the above embodiments comprises isolated mitochondria, e.g., isolated viable mitochondria, which are linked to a pharmaceutical agent, diagnostic agent, imaging agent, therapeutic agent, or any other biocompatible agent. In some further embodiments, the mitochondria are linked to an antibody or an antigen, e.g., an antibody or antigen binding fragment. In another embodiment, the mitochondria are linked to a nucleic acid, such as DNA or RNA. Other exemplary nucleic acids include, but are not limited to, double-stranded DNA, single-stranded DNA, double-stranded RNA, single-stranded RNA, or triple helix nucleic acid molecules. In certain instances, the nucleic acid polymers are DNA, interfering RNAs (siRNA), and micro RNAs. In one particular embodiment, the agent and the mitochondria are in physical contact with each other, e.g., the agent is linked to mitochondria, e.g., by a covalent bond, embedded in the mitochondria, attached to mitochondria, embedded in the mitochondrial membrane, substantially enclosed within a mitochondrion, or encapsulated entirely by mitochondria. In one other particular embodiment, the agent and the mitochondria are electrostatically linked. In one other particular embodiment, the agent is linked covalently, e.g., chemically linked, or non-covalently, e.g., electrostatically linked, to the outer membrane of the mitochondria. The mitochondria linked to an agent as described herein are referred to as “combined mitochondrial agent”.

In one aspect, the composition of the above embodiment comprises mitochondria, e.g., isolated viable mitochondria, which are linked to a therapeutic agent, which is selected from, but not limited to, the group consisting of: an inorganic or organic compound; a small molecule (less than 500 daltons) or a large molecule; a proteinaceous molecule, such as a peptide, polypeptide, protein, post-translationally modified protein, or antibody; or a nucleic acid molecule, such as a double-stranded DNA, single-stranded DNA, doublestranded RNA, single-stranded RNA, or a triple helix nucleic acid molecule. In some embodiments, a therapeutic agent is a natural product derived from any known organism (e.g., from an animal, plant, bacterium, fungus, protist, or virus) or from a library of synthetic molecules. In some embodiments, a therapeutic agent is a monomeric or a polymeric compound. Some exemplary therapeutic agents include cytotoxic agents, DNA vectors, small interfering RNAs (siRNA), micro RNAs (miRNA), reactive peptides, nanoparticles, microspheres, and fluorescent molecules. In another aspect, the composition of the any one of the embodiments above comprises mitochondria, which are linked to an imaging agent, which is a radioactive agent, fluorescent agent, or any agent that is detectable by any imaging technique such as, but not limited to, X-rays, magnetic resonance imaging (MRI), positron emission tomography (PET), computed tomography (CT), micro-computed tomography (CT), PET/CT, PET/MRI, fluorescence molecular tomography (FMT), FMT/CT, scintigraphy, or ultrasound. Exemplary imaging agents include, but are not limited to, Mito Tracker fluorophores (Thermo Fisher Scientific Inc.), CellLight® RFP, BacMam 2.0 (Thermo Fisher Scientific Inc.), pH-sensitive pHrodo fluorescent dyes (Thermo Fisher Scientific Inc.). In another aspect, the composition of any one of the embodiments above comprises mitochondria, which are linked to a diagnostic agent, e.g., fluorescents dyes or molecules containing radioactive isotope of elements, which is designed, for instance, to determine the condition within a cell, for example pH and oxidative stress within a cell.

In one aspect, the composition of any one of the embodiments above comprises isolated mitochondria, e.g., isolated viable mitochondria, which have been modified by gene editing. Non-limiting examples of gene editing techniques for the modification of mitochondrial DNA (mtDNA) are, for instance, gene editing technologies by restriction endonucleases (RE) technology, zinc finger nuclease (ZFN) technology, transcription activator-like effector nuclease (TALEN) technology, CRISPR system and pAgo-based system. Alternatively, the composition of any one of the embodiments above. comprises mitochondria, which contains exogenous mtDNA, e.g., mitochondria which have been modified by transplantation of exogenous mtDNA into autogeneic mitochondria (Trends Genet. (February 2018), 34(2): 101-110, Payam A, Gammage et al.).

In another aspect, the viable mitochondria comprised in composition according to any one of the embodiments above have been isolated according to any isolation method known in the art. In one particular embodiment, the viable mitochondria are isolated by density gradient centrifugation or ultracentrifugation, differential centrifugation, cell-specific mitochondria affinity purification (CS-MAP) (STAR Protocols, Vol. 2, Issue 4, (17 Dec. 2021), 100952, A. Ahier, T. Onraet, S. Zuryn), magnetic bead affinity purification, e.g., irreversible binding to antibody-coated magnetic beads (Hornig-Do, H. T. et al., (2009), Isolation of functional pure mitochondria by superparamagnetic microbeads. Anal. Biochem. 389: 1-5), or differential filtration (e.g., as described in Preble et al., JOVE (2014): “Rapid Isolation And Purification Of Mitochondria For Transplantation By Tissue Dissociation And Differential Filtration” and in WO 2015/192020 A1, McCully et al.). Preferably the mitochondria, e.g., viable mitochondria, are isolated by differential filtration, such as, for example, by a differential filtration run in a filtration apparatus comprising two or more filters, e.g., run in a cylindrical filtration apparatus comprising three filters, wherein the filter on the upper end of the apparatus has pores with pore size of 30 μm to 50 μm, the filter at the lower end of the apparatus has pores with a pore size of 5 μm to 20 μm and the filter placed between the upper and bottom filter has pore with a pore size of 15 μm to 50 μm. In one particular embodiment, the mitochondria comprised in the composition of any one of the embodiments above have been freshly isolated from cells or tissues prior to their incorporation (i.e., suspension) into the compositions of the invention.

### Methods

### Embodiment B: Method for the Cryopreservation of Mitochondria

The isolated mitochondria, e.g., isolated viable mitochondria, of the present invention undergo a freeze-thaw cycle. The freeze-thaw cycle comprises freezing the isolated mitochondria within (i.e., comprised in) a cryopreservative composition of any one of the cryopreservative compositions described above. Without wishing to be bound by any theory or mechanism, the possibility to freeze mitochondria contained in any one of the compositions disclosed herein and the possibility to then thaw them, without impairing their functionality and structural integrity during the freeze-thaw cycle, enables easy storage and use of the mitochondria with reproducible results even after a long period of storage.

In one aspect, the invention provides a method for the cryopreservation of a composition comprising isolated mitochondria, e.g., isolated viable mitochondria, according to any one of the embodiments above, the method comprising the steps of:
(a) freezing the composition at a temperature below 0° C.; and
(b) storing the frozen composition obtained according to step (a) at a temperature below 0° C.

In another aspect, the method according to any one of the embodiments above comprises a further step (c) subsequent to step (b), consisting in thawing the frozen composition at a temperature above 0° C.

In another aspect, the invention provides a method for the cryopreservation of a composition comprising the steps of:
(a.1) adding isolated mitochondria, such as isolated viable mitochondria, to the cryopreservative composition according to any one of the embodiments above;
(a.2) freezing the composition at a temperature below 0° C.; and
(b) storing the frozen composition obtained according to step (a.2) at a temperature below 0° C.;
(c) optionally thawing the frozen composition at a temperature above 0° C.

In another aspect, the freezing step (a) or (a.2) of the method according to any one of the embodiments above, is completed in less than 10 hours, such as 8, 6, 4, 5 or 3 hours. In one embodiment, the freezing step (a) or (a.2) is completed in less than 2.5 hours, such as, for example, 2 hours or 1.5 hours. In another embodiment, the freezing step (a) or (a.2) of the method according to any one of the embodiments above, is completed in less than 60 minutes, such as, for example less than 50, 45, or 35 minutes. In one preferred embodiment, the freezing step (a) or (a.2) is completed in less than 30 minutes, such as, for example, 25, 20, or 15 minutes. In another preferred embodiment, the freezing step (a) or (a.2) of the method according to any one of the embodiments above, is completed in less than 10 minutes, more preferably in less than 5 minutes, such as 3 or 2 minutes.

In another aspect, the freezing step (a) or (a.2) of the method according to any one of the above embodiments, is carried out at a temperature of at least −200° C. (i.e., at −200° C. or at a temperature lower than −200° C.). In some particular embodiments, the freezing step (a) or (a.2) of the method according to any one of the embodiments above is carried out at a temperature of at least (i.e., lower than) −170° C., such as in liquid nitrogen (e.g., in liquid nitrogen at a temperature of about −196° C.). In some embodiments, the freezing step (a) or (a.2) is done at a temperature of at least (i.e., lower than) −80° C., such as of at least −90° C. or −100° C. In some particular embodiments, the freezing step (a) or (a.2) of the method according to any one of the embodiments above, is done at temperature of at least (i.e., lower than) −70° C., such as at the temperature of dry ice (e.g., freezing is done in dry ice at a temperature of about −78.5° C.). In some embodiments, the freezing step (a) or (a.2) is done at a temperature of at least (i.e., lower than) −4° C. In certain embodiments, the freezing step (a) of the method of any one of the embodiments above is done at a temperature of at least 0° C. (e.g., 0° C. or below 0° C.). Preferably the freezing step (a) or (a.2) of the method according to any one of the embodiments above, is done in dry ice or in liquid nitrogen, more preferably in dry ice.

In another aspect, the freezing of the composition comprising the isolated mitochondria, e.g., isolated viable mitochondria, carried out in step (a) or (a.2) according to the methods of any one of the above embodiments, is a snap-freezing. An example of snap-freezing may be spray-freezing as described herein. In another embodiment, the freezing of step (a) or (a.2) of the method of any one of the embodiments above, is gradual. In another embodiment, the freezing of step (a) or (a.2) is gradual at a rate of at least 5° C./minute, such as 8° C./minute or 10° C./minute. In a preferred embodiment, the freezing rate of freezing step (a) or (a.2) is of at least 15° C./minutes, such as 20° C./min., 25° C./min., 30° C./min., 35° C./minute or 40° C./minute.

In one particular embodiment, the storing of step (b) of the method for cryopreserving isolated mitochondria, e.g., isolated viable mitochondria, is at a temperature below 0° C., such as, for example, −4° C., −8° C., −10° C., −15° C., −20° C., or −25° C. In one embodiment, the storing temperature of step (b) of the method of any one of the embodiments above, is at least less than −10° C., such as −20° C. or −30° C. In one embodiment the storing temperature of storing step (b) of the method of any one of the embodiments above is the temperature of dry ice (−78.5° C.). In yet another embodiment, the storing temperature of storing step (b) is the temperature of liquid nitrogen (e.g., −196° C.).

In another aspect, the storing in of step (b) according to the method of any one of the embodiments above (e.g., the conservation at low temperature of the frozen composition comprising isolated mitochondria, e.g., isolated viable mitochondria, has a duration of a period of (i.e., the storage time of the frozen composition is) at least 2 hours prior to thawing such as 3, 4, 5, 6, 7, 8, 10, 12, 15 or 18 hours. In some embodiments, the storing of step (b) has a duration of a period of at least 24 hours, such as 48, 72, 96 or 120 hours prior to thawing. In some embodiments, the storing period of step (b) is of at least 5 days (i.e., 120 hours) prior to thawing, such as 6 days (i.e., 144 hours) prior to thawing. In some other embodiments, the storing period of step (b) is of at least one week (i.e., 7 days) prior to thawing, such as 2, 3, or 4 weeks. In some other embodiments, the storing of the composition according to step (b) of the method of any one of the embodiments above has a duration of a period of at least 1 month prior to thawing, such as 3 months, 6 months, 12 months, 18 months, 24 months or longer. Each possibility represents a separate embodiment of the present invention.

In another aspect, the method according to any one of the embodiments above the step (c) consists in thawing the frozen composition at a temperature higher than 4° C., e.g., higher than 10° C. In another embodiment, the thawing of step (c), is performed at a temperature higher than 18° C., such as 20° C. or 22° C. In one particular embodiment step (c) consists in thawing the frozen composition at a temperature of no more than 40° C., such as 39° C. or 38.5° C. In one particular embodiment, the thawing of step (c) is done preferably at a temperature between 20° C. and 38° C., such as, for example at a temperature of 37° C. (e.g., in a water bath at 37° C.). In another particular embodiment, thawing is at body temperature. In another particular embodiment, thawing of step (c) is at a temperature which enables administering the mitochondria to a patient in need of treatment. In another particular embodiment, thawing of step (c) is performed gradually. For example, in some embodiments, thawing is performed at a temperature increasing at a rate of at least 1° C. per minute (i.e., 1° C./min.), such as 2° C./min., 3° C./min. or 4° C./min. In one preferred embodiment the thawing is performed at a temperature increasing at a rate of at least 5° C./min. such as 6° C./min., 7° C./min. 8° C./min. or 9° C./min., more preferably at a temperature increasing at a rate of at least 10° C./min., such as 15° C./min., 20° C./min., or 30° C./minute.

In another aspect, the thawing step (c) of the method according to any one of the embodiments above is carried out in a time period up to 6 hours, such as 5, 4 or 3 hours. In one embodiment, the thawing step (c) has a duration of up to 2.5 hours, such as 2 hours or 1.5 hours. In one particular embodiment, the thawing is carried out for a period up to 60 minutes, such 50, 45, 40, or 35 minutes. In a preferred embodiment, the thawing step (c) has a duration up to 30 minutes, such as 25 minutes or 20 minutes. In another preferred embodiment the thawing step (c) has a duration up to 15 minutes, such as 10 minutes. In a more preferred embodiment, the thawing step (c) has a time duration up to 5 minutes, such as 3 minutes.

A further method for the cryopreservation of the mitochondria comprised in the composition according to any one of the embodiments above, comprises a freezing step, in particular a spray-freezing (SF) step, which is optionally followed by a drying step (i.e., spry-freeze-drying method (SFD)). Spray-freezing (SF) and spray-freeze-drying (SFD) directly addresses the challenge of preserving the heat labile mitochondria, including mitochondrial sub-particles. Spray-freezing is applied by first spraying the composition comprising mitochondria into a liquid cryomedium (e.g., liquid nitrogen contained in a chamber or container) and then by maintaining the composition at a temperature below 0° C., such as, for example, the temperature of the cryomedium itself. The composition may be sprayed by any suitable spraying device, e.g., a spray gun, which optionally has been properly pre-cooled before use. In the SFD process, the composition is first sprayed into the cryomedium, e.g., liquid nitrogen, and frozen therein. The minimum time between the isolation of fresh mitochondria and spray-freezing of the composition comprising these isolated mitochondria, i.e. the cryopreserving composition of the present invention including isolated mitochondria, is preferably not more than 120 minutes, more preferably not more than 60 minutes, even more preferably not more than 30 minutes after the isolation of the fresh mitochondria. According to this method the freezing step requires a very short time: it is quick, nearly immediate (e.g., snap-freezing). The droplets of the sprayed composition comprising mitochondria according to any one of the above embodiments, once frozen, may be kept/stored in the cryomedium, such as liquid nitrogen, or in any other suitable freezing agent, e.g., dry-ice, for a time period of at least 2 minutes, 5 minutes, 10 minutes, 30 minutes, or 60 minutes. The droplets have a size of 1 μm-100 μm, more commonly of 3 μm-75 μm or 5 μm-55 μm. The sprayed-frozen composition may be stored at a temperature below 0° C. for at least 3 hours, 6 hours, 12 hours, 24 hours, 48 hours, 1 week, 2 weeks, 3 weeks, or 4 weeks. The sprayed-frozen composition may be stored for a period of even 1 or 2 years.

After being sprayed-frozen, the composition may be separated from the cryomedium, e.g., from the liquid nitrogen, by, for example, evaporating the cryomedium at a temperature above 0° C., e.g., at room temperature (e.g., between 20-25° C.). The evaporation of the cryomedium may be carried out under atmospheric pressure (e.g., 1 atm or 1.01325 bar) or at a pressures lower than the atmospheric pressure.

The composition obtained after having being separated from the cryomedium, e.g., after that cryomedium is removed at a pressure lower than the atmospheric pressure, may be a lyophilized composition.

The sprayed-frozen composition may be thawed before use (e.g., cryodesiccation).

In another aspect, the invention provides a method for the cryopreservation of a composition comprising isolated mitochondria, e.g., isolated viable mitochondria, according to any one of the embodiments above, the method comprising the steps of:
(i) spray-freezing the composition into a cryomedium, e.g., liquid nitrogen at a temperature of about −196° C.); and
(ii) storing the frozen composition obtained according to step (i) at a temperature below 0° C., such as at the temperature of the liquid nitrogen.

In another aspect, the invention provides a method for the cryopreservation of a composition comprising isolated mitochondria, e.g., isolated viable mitochondria, according to any one of the embodiments above, the method comprising the steps of:
(i) spray-freezing the composition into composition into a cryomedium, e.g., liquid nitrogen at a temperature of about −196° C.;
(ii) drying/evaporating the cryomedium, such as liquid nitrogen; and
(iii) storing the frozen-dried composition obtained according to step (ii) at a temperature below 0° C.

In one aspect, the invention provides a method for the cryopreservation of a composition according to any one of the embodiments above, wherein the composition comprises isolated viable mitochondria in an amount of at least 0.1 μg/μL, such as 0.2 μg/μL, 0.5 μg/μL, 0.7 μg/μL, 1 μg/μL, 1.5 μg/μL or 2.0 μg/μL, and the steps (a) and (b) of said method are:
(a) freezing the composition in liquid nitrogen (e.g., at a temperature of about −196° C.) or in dry ice (e.g., at a temperature of −78.5° C.), preferably in dry ice, for at least 1 minute; and
(b) storing the frozen composition obtained according to step (a) at a temperature below 0° C.

In one particular embodiment, the freezing step (a) or (a.2) of the method of any one of the embodiments above, is carried out in dry ice for a period of time up to 5 minutes; the storing step (b) is at a temperature below −5° C., such as, for example, at a temperature of −4° C., −8° C., −15° C., −20° C., −25° C., −50° C., or −78.5° C. and for a time period of at least 1 week, such as, 4 weeks or 8 weeks, preferably of at least 12 weeks or 24 weeks, more preferably of at least 32 weeks, such as, for example, 36 weeks or 72 weeks.

In one aspect, the method of the invention is a method for the cryopreservation of a composition according to any one of the embodiments above, wherein the composition comprises isolated mitochondria, e.g., isolated viable mitochondria, in an amount of at least 5 μg/μL, such as, for example, of at least 10 μg/μL, of at least 15 μg/μL, of at least 20 μg/μL, of at least 25 μg/μL, or at least 30 μg/μL. Yet, in a further embodiment, the invention provides a method for the cryopreservation of a composition according to any one of the embodiments above, wherein the composition comprises isolated mitochondria, e.g., isolated viable mitochondria, in an amount of at least 35 μg/μL, such as 40 μg/μL, 50 μg/μL, 60 μg/μL, 70 μg/μL or 80 μg/μL.

### Therapeutical Treatment

### Embodiment C: Cryopreservative Compositions Comprising Mitochondria for Use in the Treatment of a Disease

The present invention provides herein a cryopreservative composition comprising mitochondria, e.g., human mitochondria, or prepared by any of the above methods, for use as medicament. In particular, the present invention provides a composition according to any one of the embodiments above in a therapeutically effective amount for use in treating conditions, which benefit from increased or repaired mitochondrial function, in a subject in need thereof, as well as conditions, which benefit from the combined use of mitochondria with another pharmaceutical agent, e.g., molecules, antibodies, oligonucleotides, peptides, or extracellular vesicles. The combined use may include the concomitant application or administration of the pharmaceutical agent and the mitochondria, as well as a solution in which the pharmaceutical agent and the mitochondria are prepared as one therapeutic entity, such as for example, as one single therapeutic wherein the pharmaceutical agent has been introduced into the mitochondria or linked to the mitochondria prior to or after their cryopreservation.

The present invention provides herein a cryopreservative composition comprising isolated mitochondria, e.g., isolated viable mitochondria, according to any one of the embodiments above in a therapeutically effective amount for treating a disease or condition in a subject in need thereof. In a particular embodiment the mitochondria comprised in the composition for treating a disease are human isolated mitochondria, such as human isolated viable mitochondria, e.g., wild-type human mitochondria (e.g., naturally occurring human mitochondria), or modified human mitochondria, e.g., mitochondria modified by gene editing. In another particular embodiment, the mitochondria comprised in the composition for use in the treatment of a disease are a combined mitochondrial agent, e.g., viable mitochondria previously isolated and then linked to a pharmaceutical, therapeutic, diagnostic, or imaging agent. A combined mitochondrial agent comprises isolated mitochondria, which are combined artificially with a therapeutic agent, pharmaceutical agent, diagnostic agent, imaging agent, or any other agent. The agent is combined with a mitochondrion in any fashion, for example, linked (e.g., chemically or electrostatically linked) to a mitochondrion, attached to a mitochondrion, e.g., attached to the outer membrane of mitochondria, embedded in the mitochondrial membrane, substantially enclosed within a mitochondrion, or encapsulated entirely by a mitochondrion, as long as the mitochondrion and the agent are in physical contact with each other.

Accordingly, the present specification provides a composition according to any one of the above embodiments for use in the treatment of a disease in a patient in need by delivering said composition and the mitochondria comprised therein to the cells and/or tissues of the patient or cells derived from an allogeneic donor. The composition comprising isolated mitochondria, e.g., isolated viable mitochondria, according to any one of the embodiments above, for use in the treatment of a disease or disorder, which benefits from increased mitochondrial function, has been frozen, stored (i.e., cryopreserved) and thawed directly prior to use.

The mitochondria comprised in the composition according to any one of the embodiments above, for use in the treatment of a variety of diseases or disorders, including, but not limited to, various forms of cancer, tumors, autoimmune disease, ageing related diseases, metabolic diseases, ischemia-related or ischemia-reperfusion injuries and/or diseases, or wounds, prior to their administration to the subject in need, have been frozen, stored and then thawed according to the method for cryopreservation of any one of the embodiments above.

In one aspect, the invention provides a composition according to any one of the above embodiments, in a therapeutically effective amount for use in the treatment of a disease, e.g., by therapeutic mitochondrial transplantation (TMT), in a subject in need thereof.

In some embodiments, the mitochondria, e.g., isolated viable mitochondria, comprised in the composition according to any one of the above embodiments are autologous (i.e., autogeneic or autogenous). In some embodiments the mitochondria are autogenous or autologous mitochondria with genetic modification. In some other embodiments, the mitochondria are autologous and linked to an imaging, diagnostic or a pharmaceutical agent. In some other embodiments, the agent is embedded or incorporated into the autologous mitochondria. In some other embodiments, the mitochondria are allogeneic. In some embodiments the mitochondria are allogeneic mitochondria with genetic modification. In some other embodiments, the mitochondria are allogeneic mitochondria, which are linked to an imaging, diagnostic or a pharmaceutical agent. In some other embodiments, the agent is embedded or incorporated into the allogeneic mitochondria. In some other embodiments, the mitochondria are xenogeneic. In some embodiments the mitochondria are xenogeneic mitochondria with genetic modification. In some other embodiments, the mitochondria are xenogeneic mitochondria, which are linked to an imaging, diagnostic or a pharmaceutical agent. In some other embodiments, the agent is embedded or incorporated into the xenogeneic mitochondria.

In one aspect, the composition of any one of the embodiments above has undergone a cycle of freeze-thaw prior to use in the treatment of the disease.

In a further aspect, the composition of any of the above embodiments has undergone a freeze-thaw cycle according to the method(s) of any one of the embodiments above prior to use in the treatment of the disease.

In another aspect, the invention provides a composition comprising isolated mitochondria, e.g., isolated viable mitochondria, according to any one of the embodiments above for use in the treatment of a disease in a subject in need (e.g., a patient), said composition being administered in a therapeutically effective amount to a subject in need by various routes, e.g., by topical or parental administration, such as for example, by cutaneous or subcutaneous administration, by aerosolized administration, by direct injection, by vascular infusion, e.g., intra-venous injection (i.e., iv injection), or by injecting the composition into the blood vessel (e.g., by intra-arterial injection or infusion into the blood vessel) of the subject, wherein the blood vessel is part of the vascular system of the subject and carries blood to the target site. The blood flowing in the blood vessel carries the isolated viable mitochondria, e.g., the isolated viable mitochondria modified by gene editing, or combined mitochondrial agents to the target site, for example, an organ, a tissue, or an injured site. The target site is any part of the subject, e.g., heart, kidney, pancreas, liver, lung, bladder, gonadal glands, placenta, optic nerve, acoustic nerve, brain, or skeletal muscle. In certain embodiments, the blood vessel is the coronary artery of the subject. In some embodiments, the mitochondria or combined mitochondrial agent comprised in the composition of any one of the embodiments above are delivered to the target cells both in vitro, ex vivo and in vivo. In one particular embodiment, the composition comprising isolated viable mitochondria (e.g., mitochondria modified by gene editing or mitochondria linked to a therapeutical, diagnostic or imaging agent) according to any one of the embodiments above, is for use in the treatment of ischemia-reperfusion injury, such as lung-, kidney-, cardiac-, or brain-ischemia-reperfusion injury, said composition being administered in a therapeutically effective amount to a subject in need, for example, by direct injection or intra-venous or intra-arterial injection.

In one aspect, the composition according to any one of the embodiments above is for use in the treatment of a mitochondrial or mitochondrial-related disease in a subject in need thereof. In one embodiment, the composition according to any one of the embodiments above is for use in the treatment of mitochondrial diseases caused by mutations, e.g., acquired or inherited mutation, in mitochondrial DNA (mtDNA), or in nuclear genes that code for mitochondrial components. In another embodiment, the composition according to any one of the embodiments above is for use in the treatment of inherited mitochondrial disorders such as, but are not limited to, mitochondrial encephalopathy, mitochondrial myopathy, lactic acidosis and stroke-like episodes (MELAS) syndrome, myoclonic epilepsy with ragged red fibers (MERRF), neuropathy, ataxia and retinitis pigmentosa (NARP) syndrome, myoneurogenic gastrointestinal encephalopathy (MNGIE), Leber's hereditary optic neuropathy, myoclonic epilepsy with ragged-red fibers (MERRF) disorder, maternally inherited diabetes and deafness (MIDD), Leigh syndrome and Huntington's disease (HD). In another embodiment, the composition according to any one of the embodiments above is for use in the treatment of acquired mitochondria-related disease caused by toxicity, chemotherapy, or iatrogenic diseases. In another embodiment, the composition according to any one of the embodiments above is for use in the treatment of acquired mitochondria-related diseases or dysfunction, such as, but not limited to, central nervous system (CNS) diseases such as Alzheimer's disease, Parkinson's disease. In some embodiments, the mitochondrial and mitochondria-related diseases/disorders are chronic.

In another aspect, the composition according to any one of the embodiments above is for use in the treatment of ischemic or ischemic reperfusion related injuries, e.g., for use in facilitating the repairing process of damaged cells or tissues, in a subject in need thereof. In one embodiment, the composition according to any one of the embodiments above is for use in facilitating the repairing process of damaged cells or tissues, wherein the damage is caused by stroke, traumatic brain or spinal cord injury. In one embodiment, the composition according to any one of the embodiments above is for use in the treatment of a non-ischemic injury.

In another aspect, the composition according to any one of the embodiments above is for use in increasing the blood flow in the vascular system, for example, for angiography, in a patient in need thereof. In some embodiments, the composition is for use in dilatating vascular vessels, such as arteries or veins, of a patient in need thereof. In some other embodiments, the composition is for decreasing vascular resistance in an organ (e.g., heart, kidney, liver, or lung). In certain embodiments, the composition is for use in the localization, identification and/or removal of blockages in the blood vessels, such as, for example, a blood clot.

In another aspect, the composition according to any one of the embodiments above is for use in the treatment of wounds, e.g., to treat wounds exhibiting impaired wound healing, such as, for example, wounds in diabetic patients. In another aspect, the composition according to any one of the embodiments above is for use in the facilitation of repair response in acute wounds. In another aspect, the composition according to any one of the embodiments above is for use in the facilitation of the repair response in chronic wounds. (Arch Dermatol Res. (May 2016), 308(4):239-48, Janda J. et al.; Cell Metabolism (Dec. 7, 2021) 33, 2398-2414, Willenborg et al.; Biochem Soc Trans. (30 Oct. 2020); 48(5): 1995-2002, Horn A. et al.; Cell Regeneration volume 10, Article number: 5 (2021), Ma Y. et al.)

In another aspect, the composition according to any one of the embodiments above is for use in the treatment of aging-related changes, disorders, or diseases. Non-limiting examples of aging-related disfunctions/diseases are neurodegenerative diseases (e.g., Morbus Parkinson (PD), Alzheimer's disease (AD), dementia), skin aging, retina disfunctions, impaired hearing, age-related metabolic diseases, e.g., diabetes or cachexia, and muscles weakens, hypotonia, dystonia, dystrophy and/or atrophy (Biology (2019), 8, 48, R. H. Haas). Another example of aging-related diseases is Hutchinson-Gilford progeria syndrome, which is a progressive genetic disorder that causes children to age rapidly.

In another aspect, the composition according to any one of the embodiments above is for use in the treatment of neurodegenerative diseases, such as, for example, amyotrophic lateral sclerosis (ALS) or Multiple Sclerosis (MS).

In another aspect, the composition according to any one of the embodiments above is for use in the treatment of sarcopenia, such as ‘primary’ (or age-related) sarcopenia, which can be measured, for example, according to the diagnostic criteria of the European Working Group on Sarcopenia in Older People (EWGSOP), or for use in the treatment of ‘secondary’ sarcopenia. Non-limiting examples of secondary sarcopenia are: (a) sarcopenia resulting from bed rest, sedentary lifestyle and immobility, (b) disease-related sarcopenia, which is associated with advanced organ failure (heart, lung, liver, kidneys, brain), inflammatory diseases, malignant tumors or endocrine diseases; and (c) nutrition-related sarcopenia: results from inadequate dietary intake of energy and/or protein, such as malabsorption, gastrointestinal disorders or use of drugs that cause anorexia.

In another aspect, the composition according to any one of the embodiments above is for use in the treatment of cancer in a human subject in need thereof. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin's lymphoma (HL), hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin's lymphoma (NHL), non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer (SCLC), small intestine cancer, soft tissue sarcoma, spinal cord tumor, stomach cancer, T cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In another aspect, the composition according to any one of the embodiments above is for use in the treatment of infectious diseases in a human subject in need thereof.

In another aspect, the composition according to any one of the embodiments above is for use in the treatment or amelioration of immunological dysfunctions or diseases. In one aspect, the composition according to any one of the embodiments above is for use in mediating and regulating the immunological response. In one particular embodiment, the composition according to any one of the embodiments above is for use in the treatment or amelioration of immunological dysfunctions or diseases, wherein the mitochondria comprised in the composition as described in any one the embodiments above are transplanted into naturally occurring immune cells to produce mitochondria-enhanced immune cells. Without intending to be limited to any theory, in some embodiments, the immune system impairments (e.g., immunological disorders or dysfunctions) can be due to a decrease in immunological diversity of naïve T cells and an increasing number of senescent effector cells with age. In one particular aspect, the composition according to any one of the embodiments above is for use the treatment of autoimmune disease. Non-limiting examples of autoimmune diseases are systemic lupus erythematosus (SLE), rheumatoid arthritis, multiple sclerosis, autoimmune vasculitis, myasthenia gravis, pernicious anemia, Hashimoto's thyroiditis, type 1 diabetes, inflammatory bowel disease (IBS), autoimmune Addison's disease, Grave's disease, Sjögren's syndrome, psoriasis, and celiac diseases. Another example is the autoimmune lymphoproliferative syndrome (ALPS), which is an inherited disorder in which the body cannot properly regulate the number of immune system cells (lymphocytes). Without wishing to be bound by any theory or mechanism, mitochondria play a crucial role in regulating not only the growth, but also the function, of immune cells. In addition to providing energy to support the synthesis of the macromolecules essential for immune cell proliferation, mitochondria also act as signaling organelles, driving activation of immune cells via metabolic intermediates, mitochondrial DNA (mtDNA), and reactive oxygen species (ROS). In addition, mitochondrial dynamics (fusion and fission), biogenesis (synthesis of new mitochondria), and mitophagy (degradation of damaged mitochondria) also play important roles in regulating immune cell functions. The link between immune cell function and mitochondrial function is known in the art as “immunometabolism”. The relationship between immune and metabolic pathways is crucial in many dysfunction of the immunological responses, such as, for example, in critical illness, in particular sepsis.

In another aspect, the composition according to any one of the embodiments above is for use in gene therapies. In a certain embodiment, gene therapy is for the treatment of cancer.

In yet another particular embodiment, gene therapy is for the treatment of infectious diseases. In yet another embodiment, gene therapy is for the treatment of heart lungs, kidney, bladder, prostate, uterus, pancreas or kidney diseases. In yet another embodiment, gene therapy is for the treatment inherited gene defects or acquired genetic disorders. In a particular embodiment, gene therapy is for the treatment of autoimmune diseases. In yet another embodiment, gene therapy if for the treatment of mitochondria-related diseases. Mitochondrial diseases, such as acquired mitochondria-related diseases or dysfunction, may include, but are not limited to, central nervous system (CNS) diseases such as Alzheimer's disease, Parkinson's disease, Alpers syndrome, Leigh syndrome, and myoclonic epilepsy with ragged red fibers, as well as following stroke and traumatic brain or spinal cord injury (Mitochondrion (July 2017), 35:70-79, Gollihue and Rabchevsky). Drugs can induce mitochondrial dysfunction by different mechanisms including inhibition of fatty acid oxidation, impairment of oxidative phosphorylation and respiratory chain activity as well as alteration of the integrity of the mitochondrial membranes. Some drugs also impair mitochondrial function via the production of reactive oxygen species and the generation of reactive metabolites, which can covalently bind to key mitochondrial proteins. Drug-induced mitochondrial dysfunction plays an important role in the pathogenesis of adverse effects such as liver injury, myopathy, and cardiotoxicity. Examples of drugs, which can induce mitochondrial dysfunction are, for instance, acetaminophen, amiodarone, doxorubicin, nucleoside reverse transcriptase inhibitors (e.g., stavudine, zidovudine, didanosine), statins (e.g., atorvastatin, cerivastatin, simvastatin) and valproic acid (Mitochondrial Biology and Experimental Therapeutics, (March 2018), pp 269-295, Massart).

In one particular aspect, the composition according to any one of the embodiments above is for use in gene therapies, wherein the mitochondria comprised in the composition as described in any one the embodiments above are transplanted into naturally occurring immune cells to produce mitochondria-enhanced immune cells. In another particular embodiment, the composition according to any one of the embodiments above is for use in gene therapies, wherein the mitochondria comprised in the composition as described in any one the embodiments above are transplanted into modified immune cells, such as, but not limited to, chimeric antigen receptor (CAR) T-cell, CAR-NK cell, CAR-macrophage, neutrophil, tumor-infiltrating lymphocyte (TIL), gamma-delta T cell. In some aspects, the immune cell is a T lymphocyte, such as a CD4 T cell, e.g., a Treg cell. In some aspects, the immune cell is a natural killer (NK) cell. In some aspects, the immune cell is a mucosal associated invariant T cell. In some aspects, the immune cell is a gamma-delta T cell. In some aspects, the immune cell is a monocyte or macrophage. In some aspects, the immune cell is a neutrophil. In some aspects, the immune cell is a B lymphocyte. In some aspects, the immune cell is produced from a stem cell, a mesenchymal stem cell or an induced pluripotent stem cell (iPSC). In some aspects, the immune cell is an allogenic or autologous immune cell.

In a particular aspect, the composition according to the above embodiment is for use in gene therapies, wherein the mitochondria comprised in the composition as described in any one the embodiments above are first frozen and thawed, or frozen, stored and thawed according to a method of any one of the methods described above, and then transplanted into immune cells to produce mitochondria-enhanced immune cells, such as, but not limited to, chimeric antigen receptor (CAR) T-cell, CAR-NK cell, CAR-macrophage, neutrophil, tumor-infiltrating lymphocyte (TIL), gamma-delta T cell. In yet another aspect the mitochondria are transplanted into immune cells to produce mitochondria-enhanced immune cells before freezing.

In another aspect, the composition according to any one of the embodiments above is for use in gene therapies, wherein the mitochondria comprised in the composition as described in any one the embodiments above are transplanted into stem cells, e.g., the mitochondria of the composition are first frozen and thawed, or frozen, stored and thawed according to a method of any one of the methods described above, and then transplanted into stem cells. In some aspects, the stem cell is an embryonic stem cell. In some aspects, the stem cell is an induced pluripotent stem cell. In some aspects, the immune cell is a pluripotent stem cell-derived immune cell. In some aspects, the immune cell is a T lymphocyte, such as a helper T cell, a cytotoxic T cell, a regulatory T cell, a memory T cell. In some aspects the T lymphocyte is a CD8 T cell. In some aspects, the T lymphocyte is a CD4 T cell, such as Treg cell. In some aspects, the immune cell is a natural killer (NK) cell. In some aspects, the immune cell is a mucosal associated invariant T cell. In some aspects, the immune cell is a gamma-delta T cell. In some aspects, the immune cell is a monocyte or macrophage. In some aspects, the immune cell is a neutrophil. In some aspects, the immune cell is a B lymphocyte. In some aspects, the stem cell is an allogenic or autologous stem cell.

In a particular aspect, the composition according to the above embodiment is for use in gene therapies, wherein the mitochondria comprised in the composition as described in any one the embodiments above are first frozen and thawed, or frozen, stored and thawed according to a method of any one of the methods described above, and then transplanted into stem cells to produce mitochondria-enhanced stem cells. In yet another aspect the mitochondria are transplanted into stem cells to produce mitochondria-enhanced stem cells before the freezing cycle.

In a particular aspect, the composition according to any one of the embodiments above is for use in the treatment of acute or chronic graft-versus-host disease (GVHD).

### Terms

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The terms “include,” “such as,” and the like are intended to convey inclusion without limitation, unless otherwise specifically indicated.

As used herein, the term “or” is generally employed in its usual sense including “and/or” unless the content clearly dictates otherwise. The term “and/or” means one or all of the listed elements or a combination of any two or more of the listed elements.

As used herein, the term “comprising” also specifically includes embodiments “consisting of” and “consisting essentially of” the recited elements, unless specifically indicated otherwise.

As used herein, the term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

As used herein, the term “isolated” means altered or removed from the natural state or environment. For example, a nucleic acid, a peptide or a subcellular particle, such as a mitochondrion, naturally present in a living animal or cell is not “isolated,” but the same nucleic acid, peptide or subcellular particle, e.g., mitochondria, partially or completely separated from the coexisting materials of its natural state is “isolated.”

The “mitochondria” to be used herein refer to viable mitochondria that are (essentially) free of eukaryotic cell material, such as extraneous eukaryotic cell material, e.g. which have been isolated/purified from cells or a cell culture. Thus, only minimal amounts of cellular components (other than mitochondria) are present in (a composition of) mitochondria to be used herein. Preferably, no other cellular components than mitochondria are present in (a composition of) mitochondria to be used herein. In this sense, the “mitochondria” to be used herein are “isolated mitochondria” and the terms “mitochondria” and “isolated mitochondria” can be used interchangeably. Any current art-known technique may be used for isolation of mitochondria, such as for example, subcellular fractioning by repeated differential centrifugation (DC) or density gradient centrifugation (DGC) (Pallotti & Lenaz, 2007; Bharadwaj et al., 2015; Djafarzadeh & Jakob, 2017; Garcia-Cazarin, Snider, & Andrade, 2011; Lai et al., 2019; Alexander G. Bury et al. 2020). A preferred, non-limiting example of how to obtain “isolated mitochondria” is provided in Preble et al., JOVE (2014): “Rapid Isolation And Purification Of Mitochondria For Transplantation By Tissue Dissociation And Differential Filtration” and in WO 2015/192020 A1 (McCully et al.), wherein mitochondria are isolated by differential filtration, optionally followed by centrifugation. As used herein, the term “isolated mitochondria” refers to mitochondria separated from other cellular components of the donor cells. As used herein, the term “donor cell” refers to a cell from which the mitochondria of the invention are isolated. The terms “recipient cell”, “acceptor cell” and “host cell” are interchangeably used herein to describe a cell receiving and encompassing the isolated mitochondria.

As used herein, the term “viable mitochondria” is used throughout the specification to describe viable mitochondria, which are intact, active, functioning and respiration-competent mitochondria. According to some embodiments, “viable mitochondria” refers to mitochondria that exhibit biological functions, such as, for example, respiration as well as ATP and/or protein synthesis.

As used herein, the term “intact mitochondria” is used throughout the specification to describe mitochondria, which comprise an integer outer and inner membrane, an integer inter-membrane space, integer cristae (formed by the inner membrane) and an integer matrix. Alternatively, intact mitochondria are mitochondria which preserve their structure and ultrastructure. In another aspect, intact mitochondria contain active respiratory chain complexes I-V embedded in the inner membrane, maintain membrane potential and capability to synthesize ATP.

As used herein, the term “modified mitochondria” refers to mitochondria which have been modified by gene editing. Non-limiting examples of gene editing techniques for the modification of mitochondrial DNA (mtDNA) are, for instance, gene editing technologies by restriction endonucleases (RE) technology, zinc finger nuclease (ZFN) technology, transcription activator-like effector nuclease (TALEN) technology, CRISPR system and pAgo-based system. Alternatively, the mitochondria, e.g., the isolated viable mitochondria, which contains exogenous mtDNA, e.g., mitochondria which have been modified by transplantation of exogenous mtDNA into autologous mitochondria. Alternatively, mitochondria have been modified by the gene editor TALED technique, which, for instance, can rely on a TALE protein to target specific mitochondrial DNA sequences, and applies an enzyme that makes the desired adenine-to-guanine edit, in addition to cytosine-to-thymine reversals as well.

As used herein, the term “mitochondrial DNA (mtDNA)” refers to DNA of mitochondria, which is a double-stranded circular molecule [an outer heavy strand (H-strand) and an inner light strand (L-strand)] containing 16,569 base pairs and 37 genes (Anderson et al., 1981). There are two noncoding regions of mtDNA: one is the origin of replication of the light strand (OL), and the other is the displacement loop (D-loop), also named the control region (CR), which includes the origin of replication of the heavy strand (OH), the heavy-strand promoter, and the light-strand promoter (Lott et al., 2013). The mtDNA of mammalian cells is relatively small and genetically compact, containing two overlapping genes and very and very little noncoding sequence (Ojala et al., 1981).

As used herein, the phrase “cryoprotective composition”, “cryoprotecting composition”, “cryopreservative composition”, “cryoprotectant”, “cryoprotective agent”, “cryoprotecting agent”, or “cryopreservative agent”, refers to a chemical mixture, a chemical solution or a chemical compound which facilitates the process of cryoprotection by reducing the injury of mitochondria, e.g., isolated viable mitochondria, during freezing and thawing (e.g., during a freeze-thawing cycle). The cryoprotective agent protects mitochondria from damage associated with storage at sub-zero temperature and/or freezing, e.g., mitochondrial membrane damage due to ice crystal formation.

As used herein, the term “permeable cryoprotectant” refers to cryoprotectants, which are designed to cross biological membranes and exert their effect particularly inside the biological structure or cell. A “permeable cryoprotectant” is also referred to as “intracellular cryoprotectant”. Permeable cryoprotectants are small, non-ionic molecules with high water solubility at low temperatures and a high diffusivity for lipid membranes. This group includes propylene glycol, ethylene glycol, glycerol and dimethyl sulfoxide (DMSO). These substances diffuse highly efficiently through cell membranes and bind water inside the cell, which is withdrawn from the imagination.

As used herein, the term “cryopreserved mitochondria” or “frozen mitochondria” are mitochondria, e.g., isolate viable mitochondria, which have been preserved by cooling to a sub-zero temperature. The “cryopreserved mitochondria” includes autologous (e.g., autogeneic, or autogenous), allogeneic or xenogeneic mitochondria. The term “cryopreserved mitochondria” refers to, for example, mitochondria, which have been isolated from any type of organs, tissues, or cells, e.g., cultured cells and which have been frozen. In some particular embodiments, the frozen mitochondria are stored for a period of time. In some particular embodiments the mitochondria have undergone one or more freezing and thawing cycles. In some specific embodiments, mitochondria that have undergone a freeze-thaw cycle are viable isolated mitochondria. In some specific embodiments, cryopreserved isolated viable mitochondria are contained in (e.g., within) a cryopreservative composition. In some embodiments, mitochondria have undergone a spray-freezing step.

As used herein, the term “cryopreservative amount” or “cryoprotective amount” refers to that amount of a cryopreservative or cryoprotective compound/agent that will allow the mitochondria to retain their physical, chemical, functional and structural stability and integrity, during and after a freezing cycle or a freeze-thaw cycle. The term “less than a cryopreservative amount” refers to that amount of cryopreservative/cryoprotective compound/agent, which is not sufficient for maintaining the physical, chemical, functional, or structural stability and integrity of the mitochondria, during and after a freeze cycle of freeze-thaw cycle.

As used herein, the term “autologous”, “autogeneic”, or “autogenous” refers to any material derived from the same individual to whom it is later to be re-introduced into the individual.

As used herein, the term “allogeneic” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

As used herein, the term “xenogeneic” refers to a graft derived from an animal of a different species, i.e., “xenogeneic mitochondria” refers to mitochondria obtained by different species than the subject being treated.

As used herein, the term “freeze-thaw cycle” refers to freezing of the mitochondria of the invention to a temperature below 0° C., maintaining the mitochondria in a temperature below 0° C. for a defined period of time and thawing the mitochondria to room temperature or body temperature or any temperature above 0° C., which enables administering the mitochondria to a person in need of treatment, e.g., a person suffering, for instance, from mitochondrial or mitochondrial-related diseases or disorders, inflammatory, infectious, autoimmune diseases or disorders, cancer, ischemia or ischemia-related disorders and injuries, blood clots and blood flow dysfunctions. Each possibility represents a separate embodiment of the present disclosure. The term “room temperature”, as used herein refers to a temperature of between 18° C. and 25° C. The term “body temperature”, as used herein, refers to a temperature of between 35.5° C. and 37.5° C., preferably 37° C.

As used herein, the term “flash-freezing” or “snap-freezing” refers to rapidly freezing the mitochondria or the composition comprising mitochondria by subjecting them to cryogenic temperatures.

As used herein, the term “buffering agent”, “buffer”, “aqueous buffer”, or “buffering system” is to be understood to mean a substance that maintains the pH of an aqueous medium in a narrow range even if small amounts of acids or bases are added. The term “a buffering agent” means those single substances or combination of substances, which resist a change in hydrogen ion concentration upon the addition of acid or alkali.

As used here in the term “chelating agent” or “chelator” as used herein refers to any organic or inorganic compound that will bind to a metal ion having a valence greater than one. Exemplary chelating agent or chelator is a compound, which binds a calcium ion (i.e., calcium chelating agent or calcium chelator), e.g., EDTA and EGTA.

As used herein, the term “sugar” or “sugar component” refers to any saccharides, e.g., generic monosaccharides, disaccharides, trisaccharides, oligosaccharides, and polysaccharides. In some specific embodiments, the term sugar refers to a “modified sugar”. The term “modified sugar” refers to sugar analog that does not have the structure of a naturally occurring sugar, e.g., deoxyribose sugar or sucralose. In some specific embodiments, the term sugar refers to a “sugar derivative”. The term “sugar derivative” refers to a compound that is derived from sugar by a chemical reaction.

As used herein, the term “trehalose”, refers to a sugar consisting of two molecules of glucose. Trehalose is a disaccharide formed by a 1,1-glycosidic bond between two α-glucose units. Trehalose is also known as (2R, 3S, 4S, 5R, 6R)-2-(Hydroxymethyl)-6-[(2R, 3R, 4S, 5S, 6R)-3,4,5-trihydroxy-6-(hydroxymethyl)oxan-2-yl]oxyoxane-3,4,5-triol. Other names are α,α-Trehalose, α-d-glucopyranosyl-(1,1)-α-d-glucopyranoside, mycose or tremalose. In another embodiment, the term trehalose refers to the isomer α,β-trehalose, otherwise known as neotrehalose, and β,β-trehalose (also referred to as isotrehalose).

As used herein, the term “amino acid” refers to naturally or non-naturally occurring amino acids, as well as amino acid analogs and amino acid mimetics that function in a manner similar to the naturally occurring amino acids. The term “amino acid” refers to proteogenic or non-proteogenic amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retproteinain the same basic chemical structure as a naturally occurring amino acid. As used herein, the term “amino acid” may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “protein” refers to large biomolecules and macromolecules that comprise one or more long chains of amino acid residues. These biomolecules can be the product of either a biosynthesis or chemical (organic) synthesis techniques. The term “post-translational modified protein” is a protein which has been modified according to a “post-translational modification (PTM)”. PTM refers to the covalent and generally enzymatic Post translational modifications refer to any alteration in the amino acid sequence of the protein after its synthesis. PTM may involve the modification of the amino acid side chain, terminal amino or carboxyl group by means of covalent or enzymatic means following protein biosynthesis. Generally, these modifications influence the structure, stability, activity, cellular localization or substrate specificity of the protein. Post translational modification provides complexity to proteome for diverse function with limited number of genes modification of proteins following protein biosynthesis. Proteins are synthesized by ribosomes translating mRNA into polypeptide chains, which may then undergo PTM to form the mature protein product. PTMs are important components in cell signaling, as for example when prohormones are converted to hormones. Post-translational modifications can occur on the amino acid side chains or at the protein's C- or N-termini. They can extend the chemical repertoire of the 20 standard amino acids by modifying an existing functional group or introducing a new one, for example, by phosphorylation, glycosylation sulfation, hydroxylation, methylation, SUMOylation (i.e., the functional group is SUMO (small ubiquitin related modifier and proteins are 100 amino acid residue proteins which bind to the target protein in the same way as ubiquitin), disulfide bond formation, lipidylation, acetylation, prenylation, amongst other.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein or peptide sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein the term “polymer” refers to polymers which are suitable for clinical and medical application. The polymer is biocompatible, e.g., suitable for body and body fluids exposure. The polymer can be synthetic or natural, hydrophilic, or amphiphilic. The polymer can be biocompatible and therefore suitable for medical and clinical applications. Non-limiting examples of polymers are polyethylene glycol polymers (PEGs), which may be, but is not limited to, PEG200, PEG400, PEG550, PEG600, PEG800, PEG1000, PEG3350, PEG4000, PEG8000 or PEG10000.

As used herein, the term “antibody” is used in its broadest sense and includes certain types of immunoglobulin molecules comprising one or more antigen-binding domains that specifically bind to an antigen or epitope. The term also includes non-immunoglobulin antigen-binding protein molecules, so-called antibody mimetics. An antibody specifically includes intact antibodies (e.g., intact immunoglobulins G, IgG), antibody fragments (e.g., Fab fragment, single-chain Fv (scFv), single domain antibodies, V_{H}, V_{L}, V_{HH}, NAR, tandem scFvs, diabodies, single-chain diabodies, DARTs, tandAbs, minibodies, single-domain antibodies (e.g., camelid V_{HH}), other antibody fragments or formats known to those skilled in the art), and antibody mimetics (e.g., adnectins, affibodies, affilins, anticalins, avimers, DARPins, knottins, etc.). The antibodies can be monospecific, bi- and multi-specific.

As used herein, the term “antigen-binding domain” means the portion of an antibody or T cell receptor that is capable of specifically binding to an antigen or epitope via a variable domain. One example of an antigen-binding domain is an antigen-binding domain formed by an interface of the variable domains, V_{H} and V_{L}, of an antibody heavy and light chain, respectively. Another example of an antigen-binding domain is an antigen-binding domain formed by diversification of certain loops from the antibody mimetics. Another example of an antigen-binding domain is the variable domains of a TCR, such as TCR domains containing CDRs, e.g., αCDR1, αCDR2, αCDR3, βCDR1, βCDR2, and βCDR3.

As used herein, “variable domain” refers to a variable nucleotide sequence that arises from a recombination event, for example, it can include a V, J, and/or D region of a T cell receptor (TCR) sequence from a T cell, such as an activated T cell, or it can include a V, J, and/or D region of an antibody. The term “antigen-binding fragment” refers to at least one portion of an antibody or TCR, or recombinant variants thereof, that contain the antigen binding domain, i.e., variable domains and hypervariable loops, so-called complementarity determining regions (CDRs), that are sufficient to confer recognition and specific binding of the antigen-binding fragment to a target, such as an antigen and its defined epitope. Examples of antigen-binding fragments include, but are not limited to, Fab, Fab′, F(ab′)₂, and Fv fragments, single-chain (sc)Fv (“scFv”) antibody fragments, linear antibodies, single domain antibodies (abbreviated “sdAb”) (either V_{L} or V_{H}), camelid V_{HH} domains (nanobodies), multi-specific antibodies generated from antibody fragments, and TCR fragments. Exemplary antibody and antibody fragment formats are described in detail in Brinkmann et al. (MABS, 2017, Vol. 9, No. 2, 182-212), herein incorporated by reference for all that it teaches.

The term “scFv” refers to a fusion protein comprising a variable fragment of the antibody heavy chain (V_{H}) linked in its C-terminus with an N-terminus of a variable fragment of the antibody light chain (V_{L}) via a flexible peptide linker, and capable of being expressed as a single polypeptide chain, and wherein the scFv retains the specificity of the intact antibody from which it is derived.

As used herein, the term “antigen” or “Ag” refers to a molecule that is capable of being bound specifically by an antibody, or otherwise provokes an immune response, for example, when the antigen is processed by an antigen-presenting cell (APC). This immune response may involve either antibody production, or the activation of specific immunologically competent cells, or both.

A person skilled in the art will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated by a chemical synthesis; it can also be derived from a biological sample, or might be a macromolecule besides a polypeptide, e.g., lipid or carbohydrate. Such a biological sample can include, but is not limited to a tissue sample, a tumor sample, a cell or a fluid with other biological components.

As used herein, the term “biocompatible” refers to materials, such as, for instance, polymers, ingredient/excipients within a composition/formulation, that are non-toxic and meets standards, for example, of the U.S. Pharmacopoeia (USP) and the European Pharmacopoeia (Eur.Ph). Biocompatible materials, in biotechnology, are materials that which have no negative influence on their metabolism in direct contact with living cells, tissues, or organs.

As used herein, the term “protease”, refers to an enzyme that catalyzes (increases reaction rate or “speeds up”) proteolysis, breaking down proteins into smaller polypeptides or single amino. Examples of proteases are subtilisin, proteinase K, pepsin, trypsin, chymotrypsin, elastase, neutral protease, and others proteases alike.

As herein sued, the term “combined mitochondrial agent” refers to an isolated mitochondrion that is combined artificially with a therapeutic agent, pharmaceutical agent, diagnostic agent, imaging agent, or any other agent. The agent is combined with a mitochondrion in any fashion, for example, linked (e.g., chemically or electrostatically linked) to a mitochondrion, attached to a mitochondrion, embedded in the mitochondrial membrane, substantially enclosed within a mitochondrion, or encapsulated entirely by a mitochondrion, as long as the mitochondrion and the agent are in physical contact with each other. Combined mitochondrial agents are designed such that the mitochondrion act as a “carrier” that can transport the agent to a patient's cells/tissues, for example, after cutaneous/subcutaneous administration, aerosolized administration, or direct injection, and can release said agent (e.g., the cargo payload) into the cells/tissue. The combined mitochondrial agent can be prepared by methods including the steps of isolating mitochondria from cells or tissues and mixing the mitochondria with an effective amount of therapeutic agent, diagnostic agent, or imaging agent, under conditions sufficient to allow linkage of the therapeutic agent, diagnostic agent, or imaging agent, to the mitochondria. In some embodiments, the mitochondria are mixed with an imaging agent.

As used herein, the term “transplantation of mitochondria” or “mitochondria transplantation” or mitochondrion transplantation” or “mitochondria transfer” is used throughout the specification as a general term to describe the process of transferring at least one mitochondrion, e.g., an isolated viable mitochondrion, such as an autologous, allogeneic or xenogeneic isolated viable mitochondrion, to a recipient. The term includes all categories of mitochondria transplant known in the art, including, but not limited to, the direct (micro)injection of mitochondria into the recipient cells or tissues as well vascular infusion, e.g., intra-venous injection (i.e., iv injection), or intra-arterial injection or infusion into the blood vessel of the subject, wherein the blood vessel is part of the vascular system of the subject and carries blood to the target site.

As used herein, the term “transplantation” is used throughout the specification as a general term to describe the process of implanting an organ, tissue, mass of cells, individual cells, or cell organelles into a recipient. The term “cell transplantation” is used throughout the specification as a general term to describe the process of transferring at least one cell, e.g., an enhanced immune cell described herein, to a recipient. The terms include all categories of transplants known in the art, including blood transfusions. Transplants are categorized by site and genetic relationship between donor and recipient. The term includes, e.g., autotransplantation (removal and transfer of cells or tissue from one location on a patient to the same or another location on the same subject), allotransplantation (transplantation between members of the same species), and xenotransplantation (transplantations between members of different species).

As used herein, the term “stem cell” refers to an undifferentiated cell that can be induced to proliferate. The stem cell is capable of self-maintenance or self-renewal, meaning that with each cell division, one daughter cell will also be a stem cell. Stem cells can be obtained from embryonic, post-natal, juvenile, or adult tissue. Stem cells can be pluripotent or multipotent. The term “progenitor cell,” as used herein, refers to an undifferentiated cell derived from a stem cell, and is not itself a stem cell. Some progenitor cells can produce progeny that are capable of differentiating into more than one cell type. Stem cells include pluripotent stem cells, which can form cells of any of the body's tissue lineages: mesoderm, endoderm and ectoderm. Therefore, for example, stem cells can be selected from a human embryonic stem (ES) cell; a human inner cell mass (ICM)/epiblast cell; a human primitive ectoderm cell, a human primitive endoderm cell; a human primitive mesoderm cell; and a human primordial germ (EG) cell. Stem cells also include multipotent stem cells, which can form multiple cell lineages that constitute an entire tissue or tissues, such as but not limited to hematopoietic stem cells or neural precursor cells. Stem cells also include totipotent stem cells, which can form an entire organism. In some embodiment, the stem cell is a mesenchymal stem cell. The term “mesenchymal stem cell” or “MSC” is used interchangeably for adult cells which are not terminally differentiated, which can divide to yield cells that are either stem cells, or which, irreversibly differentiate to give rise to cells of a mesenchymal cell lineage, e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. In some embodiments, the stem cell is a partially differentiated or differentiating cell. In some embodiments, the stem cell is an induced pluripotent stem cell (iPSC), which has been reprogrammed or de-differentiated. Stem cells can be obtained from embryonic, fetal or adult tissues.

As used herein, the term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment and will be ascertainable by one skilled in the art using known techniques (see, e.g., Lieberman, Pharmaceutical Dosage Forms (vols. 1-3, 1992); Pharmaceutical Dosage Forms Disperse Systems, A. Lieberman, Martin M. Rieger, Gilbert S. Banker, 2nd Edition, (2010); Lloyd, The Art, Science and Technology of Pharmaceutical Compounding (1999); and Pickar, Dosage Calculations (1999)). Furthermore, the term “therapeutically effective amount” means any amount of mitochondria or composition comprising mitochondria, which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “therapeutic effect” refers to the effect of the therapeutic drug, substance, biological particles or composition, such as, for example, the composition comprising isolated viable mitochondria or the isolated mitochondria, which is obtained by reduction, suppression, remission, or eradication of a disease state.

The term “prophylaxis” as used herein means the prevention of or protective treatment for a disease or disease state.

As used herein, the term “subject” means a mammalian subject. Exemplary subjects include humans or mammals, such as monkeys, dogs, cats, mice, rats, cows, horses, camels, goats, rabbits, and sheep. In certain embodiments, the subject is a human. The term “subject in need”, “subject in need thereof”, “subject in need of treatment”, or “patient” is intended to include living organisms, i.e., a subject, suffering from or at risk of developing a disease, disorder, or condition or otherwise in need of the compositions and methods provided herein.

As used herein, “preventing” refers to the prevention of the disease or condition, e.g., tumor formation, in the patient. For example, if an individual at risk of developing a tumor or other form of cancer is treated with the methods of the present invention and does not later develop the tumor or other form of cancer, then the disease has been prevented, at least over a period of time, in that individual.

The term “pharmaceutical composition” refers to a preparation which is in such form as to permit the biological activity of an active ingredient and/or maintain or improve viability of a biological entity (e.g., a cell) contained therein to be effective in treating a subject, and which contains no additional components, which are unacceptably toxic to the subject in the amounts provided in the pharmaceutical composition.

The term “pharmaceutically acceptable carrier” includes saline, solvents, dispersion media, coatings, antibacterial and antifungal agents, isotonic and absorption delaying agents, and the like, compatible with pharmaceutical administration. In some embodiments, the pharmaceutically acceptable carrier is phosphate buffered saline, saline, Krebs buffer, Tyrode's solution, contrast media, or omnipaque, or a mixture thereof. The term “pharmaceutically acceptable carrier” includes also sterile mitochondria buffer (300 mM sucrose; 10 mM K⁺-HEPES (potassium buffered (4-(2-hydroxyethyl)-1-piperazineethanesulfonic acid, pH 7.2); 1 mM K⁺-EGTA, (potassium buffered ethylene glycol tetraacetic acid, pH 8.0)). The term further includes a respiration buffer (250 mM sucrose, 2 mM KH₂PO₄, 10 mM MgCl₂, 20 mM K-15 HEPES Buffer (pH 7.2), and 0.5 mM K⁺-EGTA (pH 8.0). The term further includes a T cell medium, e.g., RPMI 1640 medium GlutaMAX™ Supplement 500 ml (ThermoFisher, 61870010).

As used herein, the term “ischemia-related disease” is a disease that involves ischemia. Ischemia, as used herein, is a reduced blood flow to an organ and/or tissue. The reduced blood flow may be caused by any suitable mechanism, including a partial or complete blockage (an obstruction), a narrowing (a constriction), and/or a leak/rupture, among others, of one or more blood vessels that supply blood to the organ and/or tissue.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is cancer. In some aspects, the tumor is a solid tumor. In some aspects, the tumor is a hematological malignancy (blood tumor).

The phrase “disease associated with expression of [target]” includes, but is not limited to, a disease associated with expression of [target] or condition associated with cells which express [target] including, e.g., proliferative diseases such as a cancer or malignancy or a precancerous condition. In one aspect, the cancer is mesothelioma. In one aspect, the cancer is pancreatic cancer. In one aspect, the cancer is ovarian cancer. In one aspect, cancer is a gastric cancer. In one aspect, the cancer is lung cancer. In one aspect, cancer is an endometrial cancer. Non-cancer related indications associated with expression of [target] include, but are not limited to, e.g., autoimmune disease, (e.g., lupus, rheumatoid arthritis, colitis), inflammatory disorders (allergy and asthma), and transplantation.

The term “endogenous” refers to any material from or produced inside an organism, cell, tissue or system.

The term “exogenous” refers to any material introduced from or produced outside an organism, cell, tissue or system. In case of a patient, the term “exogenous” may refer to patient-, donor- or cell culture-derived material. For example, mitochondria isolated from the patients' muscle tissue and subsequently introduced to a population of immune cells, which may be autologous to the patient or autogenic, are considered exogenous. The term “exogenous mitochondria” refers to any mitochondria isolated from an autogenous source, an allogeneic source, and/or a xenogeneic source, wherein the source's nature may be of tissue, blood, or cultured cells.

In the context of the present invention, the following abbreviations for cryoprotective agents are used. “CRYO” refers to commercially available cryoprotectants identified by a code number. For example, CRYO33 indicates the cryoprotectant 70% w/v D-(+)-Glucose monohydrate. Examples of commercially available cryoprotectants or cryoprotecting agents are provided din Table 1b of the present specification.

As used herein, the term “parenteral” administration of a composition, e.g., a composition comprising mitochondria, refers to an administration via different routes, such as, for example, via subcutaneous (s.c.), transdermal (with systemic effect), intradermal, intraocular, intravitreal, intranasal, transmucosal, intravenous (i.v.), intramuscular (i.m.), perivascular, intra-articular, intraosseous, epidural, intratechal, intracerebral, intracerebroventricular, extra-amniotic, intrauterine, intravaginal, intracavernous, intracardiac, intravesical, intraperitoneal, intrasternal, intratumoral, or intralesional (into a skin lesion,) injections/infusions.

As used herein, the term “topical” administration” of a composition, e.g., a composition comprising mitochondria, refers to the administration of the composition or drug in a localized area of the body or to the surface and having local effect.

As used herein, the term “cancer” refers to various types of malignant neoplasms, most of which can invade surrounding cells, tissues, or organs, and may metastasize to different sites, as defined by Stedman's Medical Dictionary 25′ edition (Hen syl ed. 1990). Examples of cancers which may be treated by the present invention include, but are not limited to, brain, ovarian, colon, prostate, kidney, bladder, breast, lung, oral and skin cancers. Other examples of cancer are mesothelioma, papillary serous ovarian adenocarcinoma, clear cell ovarian carcinoma, mixed Mullerian ovarian carcinoma, endometroid mucinous ovarian carcinoma, malignant pleural disease, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, or breast adenocarcinoma.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Examples

The following are examples of methods and compositions of the invention. It is understood that various other embodiments may be practiced, given the general description provided herein.

### Abbreviations

TABLE 1b

| | |
|---|---|
| CRYO1 | 100% (+/−)-2-Methyl-2,4-pentanediol |
| CRYO2 | 6.0M 1,6-Hexanediol |
| CRYO3 | 100% 1,2-Propanediol |
| CRYO4 | 100% 2,3-Butanediol |
| CRYO5 | 50% w/v NDSB-201 |
| CRYO6 | 6.0M L-Proline |
| CRYO7 | 4.0M Trimethylamine N-oxide dihydrate |
| CRYO8 | 100% Glycerol |
| CRYO9 | 100% Ethylene glycol |
| CRYO10 | 50% v/v Diethylene glycol |
| CRYO11 | 100% Polyethylene glycol 200 |
| CRYO12 | 100% Polyethylene glycol 400 |
| CRYO13 | 100% Polyethylene glycol monomethyl ether 550 |
| CRYO14 | 80% v/v Polyethylene glycol 600 |
| CRYO15 | 50% w/v Polyethylene glycol 1,000 |
| CRYO16 | 50% w/v Polyethylene glycol 3,350 |
| CRYO17 | 50% w/v Polyethylene glycol 4,000 |
| CRYO18 | 50% w/v Polyethylene glycol monomethyl ether 5,000 |
| CRYO19 | 50% w/v Polyethylene glycol 8,000 |
| CRYO20 | 50% w/v Polyethylene glycol 10,000 |
| CRYO21 | 50% w/v Polyvinylpyrrolidone K 15 |
| CRYO22 | 50% v/v Pentaerythritol propoxylate (5/4 PO/OH) |
| CRYO23 | 100% Polypropylene glycol P 400 |
| CRYO24 | 100% Dimethyl sulfoxide (DMSO) |
| CRYO25 | 70% w/v D-(+)-Sucrose |
| CRYO26 | 70% w/v D-Sorbitol |
| CRYO27 | 30% w/v D-(+)-Maltose monohydrate |
| CRYO28 | 35% w/v meso-Erythritol |
| CRYO29 | 70% w/v Xylitol |
| CRYO30 | 15% w/v myo-Inositol |
| CRYO31 | 20% w/v D-(+)-Raffinose pentahydrate |
| CRYO32 | 50% w/v D-(+)-Trehalose dihydrate |
| CRYO33 | 70% w/v D-(+)-Glucose monohydrate |
| CRYO34 | 5.0M Lithium acetate dihydrate |
| CRYO35 | 10.0M Lithium chloride |
| CRYO36 | 4.0M Lithium formate monohydrate |
| CRYO37 | 8.0M Lithium nitrate |
| CRYO38 | 2.0M Lithium sulfate monohydrate |
| CRYO39 | 3.4M Sodium malonate pH 7.0 |
| CRYO40 | 3.4M Magnesium acetate tetrahydrate |
| CRYO41 | 5.0M Sodium chloride |
| CRYO42 | 7.0M Sodium formate |
| CRYO43 | 7.0M Sodium nitrate |
| CRYO44 | 100% Tacsimate ™ pH 7.0 |
| CRYO45 | 1.0M Sodium sulfate decahydrate |
| CRYO46 | 50% v/v Ethylene glycol, 25% w/v NDSB-201 |
| CRYO47 | 30% w/v Polyethylene glycol 3,350, 20% v/v Glycerol |

In Table 1b above are provided example of commercially available cryoprotecting agents.

The cryoprotecting agents described in Table 1b have been used for the preparation of the composition by diluting the solution at 5%, 10% and 20% (v/v) (see FIG. 3A and FIG. 3B and CRYO1-CRYO47 of Table 1b), at 5%, 10% 15%, 20%, 25% and 30% (v/v) (see FIG. 3C for CRYO6, CRYO12, CRYO15, CRYO22, CRYO25, and CRYO33) and at 10%, 20%, and 30% (v/v) (FIGS. 4A-D for CRYO6+CRYO12, CRYO6+CRYO25, CYRO6+CRYO33 and CRYO33+CRYO25) in mitochondrial “trehalose-based” isolation buffer (1) below.

Preferred cryoprotectants are: CRYO13, CRYO14, CRYO15, CRYO16, CRYO17, CRYO18, CRYO22, CRYO23, CRYO25, CRYO38, CRYO46, and/or CRYO47. More preferably the composition comprises a cryoprotectant selected from the group consisting of CRYO6, CRYO12, CRYO25 and/or CRYO33.

### Isolation Buffers

(1) “trehalose-based” aqueous buffer: 10 mM HEPES, 1 mM EGTA, 300 mM trehalose; or
(2) “sucrose-based” aqueous buffer: 10 mM HEPES, 1 mM EGTA, 300 mM sucrose

### Example 1a: Isolating Mitochondria from Tissue Samples or Cultured Cells

Experiments were performed to isolate mitochondria from tissue samples.

### Preparation

The following solutions were prepared to isolate intact, viable, respiration-competent mitochondria. To successfully isolate mitochondria using the present methods, solutions and tissue samples should be kept on ice to preserve mitochondrial viability. Even when maintained on ice, isolated mitochondria will exhibit a decrease in functional activity over time (Olson et al., J Biol Chem 242:325-332, 1967). The following solutions should be prepared in advance if possible:
1 M K-HEPES Stock Solution (adjust pH to 7.2 with KOH).
0.5 M K-EGTA Stock Solution (adjust pH to 8.0 with KOH).
1 M MgCl₂ Stock Solution (to be used if a ionic component shall be in the composition).
1×PBS (ThermoFisher, 10010031)
Subtilisin A Stock was prepared by weighing out 2 mg of Subtilisin A into a 1.5 mL microfuge tube. Stored at −20° C. until use. Prepared at 2 mg/ml in isolation buffer.

With the above stock solutions, the isolation buffer at pH 7.2, consisting of 300 mM sucrose, 10 mM K-HEPES, and 1 mM K-EGTA, was prepared and stored at 4° C.

Isolation of Mitochondria from Tissue

Two, 6 mm biopsy fresh sample punches taken from the skeletal muscles were transferred to 5 mL of isolation buffer in a gentleMACS C Tube (Miltenyi Biotec, Somerville, MA) and the samples were homogenized using the gentleMACS™ Dissociator's (Miltenyi Biotec) 1-minute homogenization program. Subtilisin A stock solution (250 μL) was added to the homogenate in the gentleMACS C tube and incubated on ice for 10 minutes. The homogenate was centrifuged at 750×g for 4 minutes (as an optional step). Afterwards, the homogenate was filtered through a pre-wetted 40 μm mesh filter in a 50 mL conical centrifuge tube on ice. The filtrate was re-filtered through a new pre-wetted 40 μm mesh filter in a 50 mL conical centrifuge on ice. The filtrate was re-filtered again through a new pre-wetted 10 μm mesh filter in a 50 mL conical centrifuge tube on ice. The filtrate was re-filtered through a new pre-wetted 6 μm mesh filter in a 50 mL conical centrifuge tube on ice. The resulting filtrate was transferred to 1.5 mL microfuge tubes and centrifuged at 9000×g for 10 minutes at 4° C. The supernatant was removed, and the pellets containing mitochondria were re-suspended, and combined in 1 mL of isolation buffer.

### Example 1b: Isolating Mitochondria from Cultured Cells

Experiments were performed to isolate mitochondria from cultured cells.

### Preparation

The following solutions were prepared to isolate intact, viable, respiration-competent mitochondria. To successfully isolate mitochondria using the present methods, solutions and tissue samples should be kept on ice to preserve mitochondrial viability. Even when maintained on ice, isolated mitochondria will exhibit a decrease in functional activity over time (Olson et al., J Biol Chem 242:325-332, 1967). The following solutions should be prepared in advance if possible:
1 M K-HEPES Stock Solution (adjust pH to 7.2 with KOH).
0.5 M K-EGTA Stock Solution (adjust pH to 8.0 with KOH).
1×PBS (ThermoFisher, 10010031)
Subtilisin A Stock was prepared by weighing out 2 mg of Subtilisin A into a 1.5 mL microfuge tube. Stored at −20° C. until use. Prepared at 2 mg/ml in isolation buffer.
The above stock solutions were used to prepare the isolation buffer (pH 7.2): 300 mM sucrose, 10 mM K-HEPES, and 1 mM K-EGTA. Stored at 4° C.
Trypsin (catalog number 0103; ScienCell Research Laboratories, Carlsbad, CA).

With the above stock solutions, the isolation buffer at pH 7.2, consisting of 300 mM sucrose, 10 mM K-HEPES, and 1 mM K-EGTA, was prepared and stored at 4° C.

Isolation of Mitochondria from Cultured Cells

Mitochondria were also isolated from the cultured cells, for example, from human cardiac fibroblast (HCF) cell line (obtained from ScienCell Research Laboratories, Carlsbad, CA).

### Culture of the Human Cardiac Fibroblast (HCF) Cells

Human cardiac fibroblasts (HCF) were maintained in Fibroblast Medium-2 containing fetal bovine serum, fibroblast growth supplement-2, and antibiotic (penicillin/streptomycin) solution according to the supplier's directions (ScienCell). The cells were maintained as a monolayer at 37° C. in humidified atmosphere of 5% CO₂ and were passaged when 80% confluence was reached.

### Preparation of the Human Cardiac Fibroblast (HCF) Cells

HCF cells from two flasks (T150) at a confluency of 80% were washed once with PBS. Then trypsin was used to detach the cells according to the supplier instructions (ScienCell Research Laboratories, Carlsbad, CA). The reaction was stopped by adding trypsin neutralizing solution according to the supplier's instructions (ScienCell Research Laboratories, Carlsbad, CA). The cells were collected in a 50 mL centrifuge tube and centrifuged for 5 minutes at 1000 rpm (190×g). The supernatant was discarded and three washes with 1×PBS were performed in total.

Preparation of culture cells different from HCF, should be done according to the manufacturer's instructions. Of note, the cells used as the source of mitochondria can be adherent, semi-adherent or in suspension.

Isolation of Mitochondria from HCF Cultured Cells

The HCF cells from each flask were transferred to 5 mL of isolation buffer (i.e., the “sucrose-based” isolation buffer) in a gentleMACS C Tube (Miltenyi Biotec, Somerville, MA) and the samples were homogenized using the gentleMACS™ Dissociator's (Miltenyi Biotec) 1-minute homogenization program. Subtilisin A stock solution (250 μL) was added to the homogenate in the gentleMACS C tube and incubated on ice for 10 minutes. The homogenate was filtered through a pre-wetted 40 μm mesh filter in a 50 mL conical centrifuge tube on ice. The filtrate was re-filtered through a new pre-wetted 40 μm mesh filter in a 50 mL conical centrifuge on ice. The filtrate was re-filtered again through a new pre-wetted 10 μm mesh filter in a 50 mL conical centrifuge tube on ice. Optionally, the filtrate was re-filtered again through a new pre-wetter 5 μm mesh filter in a 50 mL conical centrifuge tube on ice. The resulting filtrate was concentrated by centrifugation. After centrifugation, the filtrate was transferred to 1.5 mL microfuge tubes and centrifuged at 9500×g for 5 minutes at 4° C. Three washes were performed at the same centrifugation speed.

### Quantification of Isolated Mitochondria

The isolated mitochondria were suspended in the isolation buffer of Example 1b and kept on ice until use. Mitochondria quantity, in preparation for varying dosage administration, was measured using a Qubit™ Fluorometer (ThermoFisher Scientific/Invitrogen), employing the Qubit™ Protein Assay kit in accordance with the manufacturer's instructions. For the protein concentration measurement, the mitochondria were resuspended in PBS (ThermoFisher, 10010031). The mitochondria dosage was estimated in terms of protein content expressed in μg.

### Example 2: Viability of Frozen-Thaw Mitochondria

Experiments were performed with isolated mitochondria to assess their functional state after freezing and thawing under different conditions.

### 2.1 Mitochondrial Isolation

Mitochondria were isolated as in Example 1b, with minor modifications described below. HCF cells were cultured in FM-2 medium (ScienCell) in T150 flasks (Sarstedt) at 37° C., 5% CO₂. Cells from two T150 flasks at 90% confluency were collected by trypsinization, washed twice with Gibco PBS pH 7.4 (Fisher Scientific, Cat. Nr. 10010023), homogenized using the gentleMACS™ Dissociator's (Miltenyi Biotec) 1-minute homogenization program, and incubated with 0.1 mg/ml subtilisin A (Protease from Bacillus licheniformis, Sigma-Aldrich) for 10 minutes on ice in isolation buffer containing either sucrose (10 mM HEPES pH 7.2, 1 mM EGTA, 300 mM sucrose) or trehalose (10 mM HEPES pH 7.2, 1 mM EGTA, 300 mM trehalose). The resulting cell lysates were filtered through two 40 μm (Falcon® 40 μm Cell Strainer, Corning Cat. Nr. 352340) and one 10 μm filter (pluriStrainer® 10 μm (Cell Strainer), PluriSelect Cat. Nr. 43-50010-03), and the mitochondria were pelleted at 9500 g for 5 minutes. Additional spin at 500 g for 5 minutes, 4° C., was performed to remove unopened cells. Protein concentration in the mitochondrial suspension was determined by Qubit Protein assay (Thermo Fisher) as described in Example 1b. Mitochondria were pelleted again, washed twice with isolation buffer.

### 2.2 Freezing of Isolated Mitochondria

Isolated mitochondria were frozen under different conditions (i.e., liquid nitrogen or dry ice) and thawed for subsequent functional analysis.

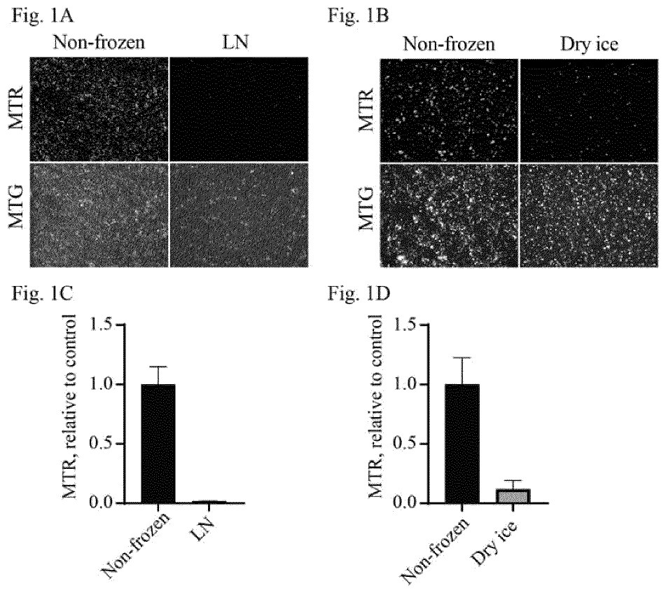

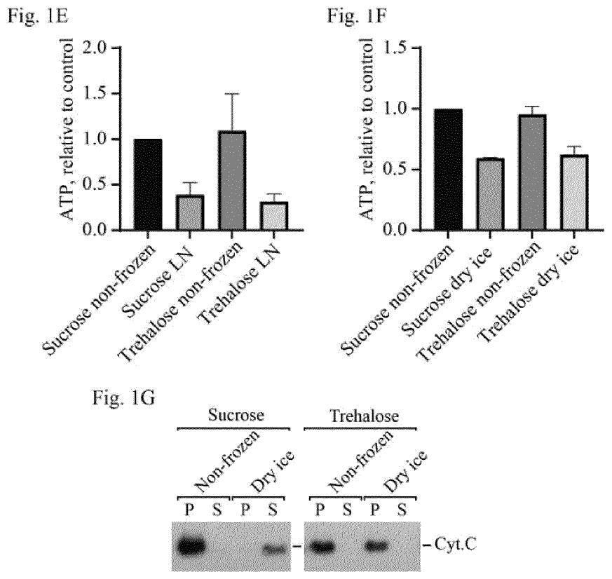

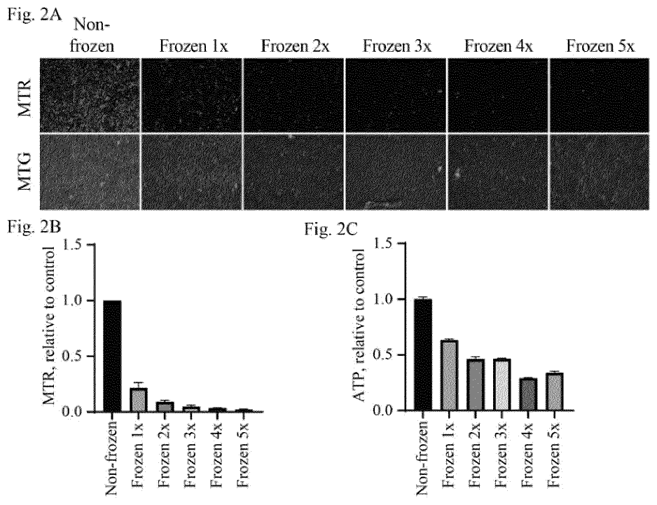

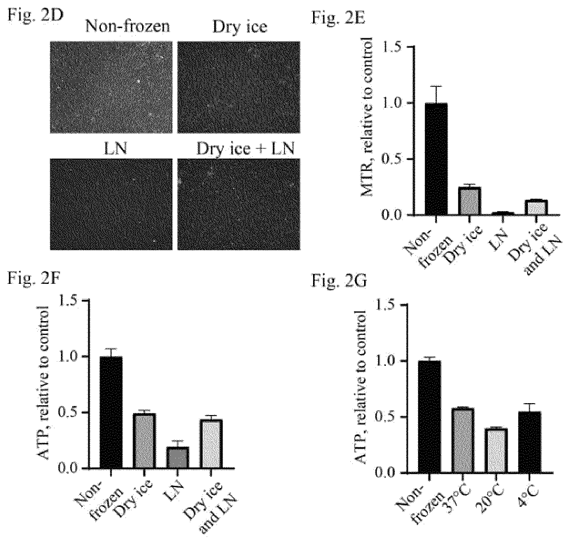

Procedure: Isolated mitochondria in the form of pellets were resuspended in two different isolation buffers containing respectively either 300 mM sucrose or 300 mM trehalose and then frozen in 1.5 ml Eppendorf tubes by placing them either in liquid nitrogen for approximately 1 minute or on dry ice for approximately 3 minutes. For thawing, the tubes were placed in a 37° C. water bath until the entire sample was thawed, and then immediately transferred on ice. Subsequently, thawed isolated mitochondria were compared to non-frozen controls using membrane potential assay, ATP assay, or by assessing cytochrome c release (FIG. 1).

### 2.3 Mitochondrial Membrane Potential Assay

To assess the mitochondrial membrane potential, the isolated mitochondria were resuspended either in the sucrose-containing isolation buffer or in the trehalose-containing isolation buffer at 1 mg/ml, and 15 μg of mitochondrial suspension were used for each sample. Both non-frozen mitochondria and mitochondria frozen and thawed according to procedure described under section 2.2 were stored on ice until use, e.g., stored for a period of 5 minutes to maximum 60 minutes, preferably for a few minutes (e.g., for a period of maximum 15 minutes, such as 5 minutes or 10 minutes). For Mitotracker Red CMXRos/Mitotracker Green FM staining, a labelling solution was prepared, by adding 200 nM MitoTracker Red CMxROS and 200 nM MitoTracker Green FM (Thermo Fisher) in the isolation buffer. The isolated mitochondria were then suspended in 1 ml of the labelling solution, vortexed and incubated for 15 minutes in a 37° C. incubator. Mitochondria were sedimented by centrifugation at 9500 g for 5 minutes at 4° C., washed once with the respective isolation buffer, and resuspended in the isolation buffer at a concentration of 1 mg/ml. Subsequently, 15 μl of mitochondrial suspension were mixed with 15 μl of 40% PEG 8000, and MitoTracker Red (579/599 nm) and MitoTracker Green (490/516 nm)-labelled mitochondria were imaged using automated microscope Keyence BZ-8000 by taking 3 images/sample at different positions. MitoTracker Red or MitoTracker Green signal was quantified using Keyence BZ-800 Analyzer software, integrated brightness per image was taken for calculations and averaged between images taken from one sample.

Result: This experiment showed that mitochondria frozen in both the sucrose-containing isolation buffer and trehalose-containing isolation buffer do not maintain the inner membrane potential (FIG. 1A, FIG. 1B, FIG. 1C and FIG. 1D).

### 2.4 ATP Assay

ATP measurement was performed using ATPlite Luminescence Assay System (catalog number: 6016941, Perkin Elmer), according to the manufacturer's instructions. 10 μg of mitochondria suspended in isolation buffer (i.e., either in the “sucrose-based” or in the “trehalose-based” buffers) were used per sample. The non-frozen mitochondria (control sample) were kept on ice. Mitochondria were frozen by placing them on dry ice, thawed on a 37° C. water bath, pelleted at 4° C., 5 min, 9500 g, resuspended in 50 μl of isolation buffer and transferred to OptiPlate-96, White Opaque 96-well Microplate (catalog number: 6005290; Perkin Elmer). 25 μl of Mammalian cell lysis solution (i.e., the solution as provided in the ATPlite Luminescence Assay, Perkin Elmer) were added, and the plates were shaken at 700 rpm for 5 minutes. After that, 25 μl of the Lyophylized substrate solution dissolved in ATPlite buffer (as provided in the ATPlite Luminescence Assay, Perkin Elmer) were added, followed by further 5 minutes of shaking at 700 rpm. The plates were kept in the dark, and luminescence was measured 10 minutes after mixing. The values were normalized to control, and results presented as ATP content relative to control.

Result: This experiment showed that mitochondria frozen in both the sucrose-containing isolation buffer and trehalose-containing isolation buffer do not maintain their ATP content when compared to the freshly isolated mitochondria (FIG. 1E and FIG. 1F).

### 2.5 Cytochrome c Release Assay

Cytochrome c release was assessed by separating mitochondria-bound cytochrome c from released cytochrome c and analyzed by western blotting. Mitochondria isolated either in sucrose-based buffer or trehalose-based buffers were frozen/thawed on dry ice/37° C. water bath, incubated for 15 minutes at 37° C., and pelleted at 20000 g for 20 minutes. Supernatant was separated from the pellet and transferred to clean tubes. Both pellet and supernatant samples were lyzed in a final volume of 20 μl of 1× NuPage loading buffer (Thermo Fisher, Cat. Nr NP0008) and analysed using NuPAGE system with Bolt™ 12%, Bis-Tris, 1.0 mm, Mini Protein Gel, 15-well (Thermo Fisher, NW00125BOX) and NuPAGE™ MES SDS Running Buffer (20×) (Thermo Fisher, NP0002). Proteins were transferred to Power Blotter Pre-cut Membranes and Filters, PVDF, regular size (Thermo Fisher PB9320) using wet blotting technique with Mini-PROTEAN® Tetra system (BioRad Cat. Nr. 1703930), blocked with 5% skimmed milk dissolved in phosphate-buffered saline (Thermo Fisher Cat. Nr. 14190144) with 0.1% Tween-20, and cytochrome c was detected using primary anti-cytochrome c antibody (Proteintech Cat. Nr. 10993-1-AP) and secondary (SA00001-2) antibody with SuperSignal™ West Pico PLUS Chemiluminescent Substrate (Thermo Fisher Cat. Nr. 34577) and CL-XPosure™ Film, 5×7 in. (13×18 cm) (Thermo Fisher Cat. Nr. 34090) films.

Results: Mitochondria frozen-thawed in sucrose-containing isolation buffer do not maintain outer membrane integrity, whereas Mitochondria frozen-thawed in trehalose-containing isolation buffer do maintain outer membrane integrity (FIG. 1G).

### Example 3: Effect of the Freezing and Thawing Rates on Mitochondria Viability

Mitochondria were isolated as described in Example 2.1 and treated as described in Example 2.2, except for that only trehalose-based isolation buffer (10 mM HEPES-KOH, pH 7.2, 1 mM EGTA, 300 mM trehalose) was used in all experiments. Mitochondrial membrane potential was assessed by MitoTracker staining, and ATP content was determined as described in Example 2.3 and 2.4.

First, mitochondria were repeatedly frozen/thawed on dry ice/37° C. water bath and tested by MitoTracker staining and ATP assay, as described in Example 2. Next, mitochondria were frozen under different conditions (i.e., by placing them either on dry ice, in liquid nitrogen, or on dry ice first and transferring to liquid nitrogen afterwards). Mitochondrial viability was estimated using MitoTracker staining and ATP assay as in Examples 2.3 and 2.4. Finally, isolated mitochondria were frozen on dry ice and thawed either in a 37° C. water bath, or at room temperature, or by placing them on ice, and ATP content was determined using ATP assay.

Results: Mitochondria which were frozen-thawed in trehalose-containing isolation buffer resulted to be functionally and structurally damaged at different degrees, depending on the freezing temperature rate and thawing temperature (FIG. 3A-3G).

### Example 4: Viability of Frozen-Thawed Mitochondria Comprised in a Composition Containing One or More Cryoprotectants

### 4.1 Integrity of the Inner Mitochondrial Membrane of Frozen-Thawed Mitochondria

The mitochondria isolated according to Example 2.1 were resuspended in the “trehalose-based” isolation buffer. The effect of cryoprotectant addition on maintaining integrity of the inner mitochondrial membrane was studied using MitoTracker staining assay, as described in Example 2.3. The mitochondria were frozen on dry ice after the addition to the mitochondria of a single cryoprotectant selected from the list of cryoprotectants provided in Table 1b, the cryoprotectant having three different concentrations of 5% (v/v), 10% (v/v), and 20% (v/v). The mitochondria of a control sample were frozen on dry ice without any cryoprotectant, and non-frozen mitochondria were kept on ice (4° C.). Alle frozen samples were thawed in a 37° C. water bath and analyzed by MitoTracker staining. MitoTracker Red signal was quantified and normalized to the signal of a non-frozen control and the results are shown in the form of a heat map in FIG. 3A. The Mito Tracker Red/Green staining and MitoTracker Red/Green signal assays of four selected cryoprotectants (i.e., CRYO12, CRYO06, CRYO33 and CRYO25) are shown in FIG. 3B.

Results: The Mito Tracker Red staining assays show that the cryoprotectants CRYO12, CRYO06, CRYO33 and CRYO25 preserve mitochondria inner membrane integrity.

### 4.2 ATP Content of Frozen-Thawed Mitochondria.

4.2.1 Use of one single cryoprotectant. Different cryopreservation compositions with increasing concentrations of one single cryoprotectant selected from the group consisting of proline, sucrose, glucose, PEG400, PEG1000 and pentaerythritol propoxylate were prepared by adding CRYO6, CRYO12, CRYO15, CRYO22, CRYO25, CRYO33 to mitochondria resuspended in trehalose-based isolation buffer. The obtained samples were then frozen on dry ice (−78° C. ca.) and thawed on a water bath at 37° C. ATP content was determined relative to non-frozen controls (kept on ice at 4° C. ca.).

Results: The inner mitochondrial membrane potential and the ATP content are shown to be dependent on the type and concentration of cryoprotectant (FIG. 3C).

4.2.2 Use of two cryoprotectants. Different cryopreservation compositions with increasing concentrations of one of the two cryoprotectants contained in each composition were prepared. The cryoprotectant were selected from the group consisting of proline, sucrose, glucose or PEG400.

Each composition comprises isolated viable mitochondria, a buffer consisting of 10 mM HEPES-KOH (pH 7.2), a calcium chelator consisting of 1 mM EGTA, trehalose at a concentration of 300 mM, and one or two cryoprotecting agents at specific concentrations as elucidated in Table 2.

TABLE 2

| | CRYO6 | CRYO12 | CRYO25 | CRYO33 |
|---|---|---|---|---|
| FIGS. 4A, 4B, 4C | 1.2M | — | — | — |
| (20% CRYO6) | | | | |
| FIG. 4A | 1.2M | 10% (w/v) | — | — |
| (20% CRYO6 + | | | | |
| 10% CRYO12) | | | | |
| FIG. 4A | 1.2M | 20% (w/v) | — | — |
| (20% CRYO6 + | | | | |
| 20% CRYO12) | | | | |
| FIG. 4A | 1.2M | 30% (w/v) | — | — |
| (20% CRYO6 + | | | | |
| 30% CRYO12) | | | | |
| FIG. 4B | 1.2M | — | 204.4 mM | — |
| (20% CRYO6 + | | | | |
| 10% CRYO25) | | | | |
| FIG. 4B | 1.2M | — | 408.8 mM | — |
| (20% CRYO6 + | | | | |
| 20% CRYO25) | | | | |
| FIG. 4B | 1.2M | — | 613.2 mM | — |
| (20% CRYO6 + | | | | |
| 30% CRYO25) | | | | |
| FIG. 4C | 1.2M | — | — | 353 mM |
| (20% CRYO6 + | | | | |
| 10% CRYO33) | | | | |
| FIG. 4C | 1.2M | — | — | 706 mM |
| (20% CRYO6 + | | | | |
| 20% CRYO33) | | | | |
| FIG. 4C | 1.2M | — | — | 1059 mM |
| (20% CRYO6 + | | | | |
| 30% CRYO33) | | | | |
| FIG. 4D | — | — | — | 706 mM |
| (20% CRYO33) | | | | |
| FIG. 4D | — | — | 204.4 mM | 706 mM |
| (20% CRYO33 + | | | | |
| 10% CRYO25) | | | | |
| FIG. 4D | — | — | 408.8 mM | 706 mM |
| (20% CRYO33 + | | | | |
| 20% CRYO25) | | | | |
| FIG. 4D | — | — | 613.2 mM | 706 mM |
| (20% CRYO33 + | | | | |
| 30% CRYO25) | | | | |

Then these compositions were added to isolated mitochondria (10 μg per sample, in triplicates, as described in Example 2.4), which have been previously suspended in a trehalose-containing isolation buffer, as described in Example 2. The obtained samples were then frozen on dry ice (−78° C. ca.) and thawed on a water bath at 37° C. ATP content was determined relative to non-frozen controls (kept on ice (4° C. ca.)). ATP assay was performed according to the procedure provided in the Example 2.

Results: The inner mitochondrial membrane potential and the ATP content are shown to be dependent on the type and concentration of cryoprotectant (FIG. 4A-4D).

### Example 5: Effect of Controlled Freezing Rates on Mitochondria Viability

Mitochondria were isolated from HCF cells as described in Example 2 and resuspended in trehalose-based isolation buffer supplemented with cryoprotectants (10 mM HEPES, pH 7.2, 1 mM EGTA, 300 mM trehalose, 1.2 M proline, 353 mM D-glucose). Protein content was determined by Qubit assay as described in Example 1b. 100 μg of the mitochondria were transferred to a 1.5 ml Eppendorf tube and frozen by placing them in a −80° C. freezer. These samples were later used for normalization of mitochondrial content between different mitochondrial isolations. Remaining mitochondria were diluted with the trehalose-based isolation buffer supplemented with cryoprotectants (10 mM HEPES, pH 7.2, 1 mM EGTA, 300 mM trehalose, 1.2 M proline, 353 mM D-glucose) to a concentration of 0.166 μg/μl. Each 50 μl of this mitochondrial suspension were filled into twelve wells of a Greiner 96 well pFT flat bottom microplate (i.e., 100 μg of mitochondrial suspension in a plate). The microplate was mounted to an ILK multiwellRACK heat exchanger and frozen in a SY-LAB IceCube 15 M controlled rate freezer by the use of a sample-specific temperature program. After pre-cooling to −5° C. within 17 minutes, ice nucleation was mechanically triggered and freezing was performed with a sample-specific cooling rate of either 2±0.1 K/min, 6±0.3 K/min or 10±0.5 K/min (i.e., Kelvin/minute). Sample-specific temperature programs were developed by an iterative adjustment of the IceCube's temperature envelope to the desired sample freezing rates monitored at different positions of the microplate using 20 Omega TJC2-NNIN-IM050U-800 0.50 mm type N thermocouples operated by a Delphin ExpertKey 200 measuring system. Frozen plates were stored at −80° C. for one month, and then analyzed using ATP assay and MitoTracker staining, as described in Example 2. ATP content was also determined in samples frozen in Eppendorf tubes at −80° C., to normalize for differences in mitochondrial amount caused by variations between mitochondrial isolations. For MitoTracker Red/MitoTracker green staining, fresh mitochondrial isolation was performed, and mitochondrial staining intensities were compared between fresh mitochondria and mitochondrial frozen at controlled rates and stored for one month.

Results: The results demonstrate that mitochondria frozen at a rate of 10 K/min have a higher ATP content and higher membrane potential compared to mitochondria frozen at slower freezing rates (2 K/min and 6 K/min). In addition, this experiment shows that the composition comprising the isolated viable mitochondria can be frozen with the method described in Example 5 and stored for at least one month.

### Example 6: In Vivo Coronary Blood Flow Experiment

Mitochondria were isolated as described in Example 2, resuspended at a concentration of 10 mg/ml in a cryopreservation buffer (7 mM HEPES, pH 7.2, 0.7 mM EGTA, 210 mM trehalose, 1.2 M proline, and 353 mM D-glucose), and frozen in 1.5 ml Eppendorf tubes in 500 ug aliquots by placing the tube on dry ice and storing it subsequently at −80° for a period of up to one week. For each in vivo experiment, the desired number of aliquots (indicated in the example) was thawed in a 37° C. water bath and immediately diluted in 6.5 ml of sucrose-based isolation buffer. Mitochondria were injected intracoronary into the heart of a healthy anesthesized pig (Sus scrofa) within 5 minutes of thawing. In certain experiments (as indicated in the example), mitochondria were freshly isolated from HCF cells (as described in Example 2) or from skeletal muscle tissue of an animal (as in Example 1a), stored on ice and subsequently injected exactly as described above for frozen mitochondria.

Results: In the preclinical test in pig the composition comprising isolated mitochondria frozen under the conditions described in Example 6 have the same effect on coronary blood flow as freshly isolated mitochondria.

### Example 7: Mitochondrial Transfer Increases the Killing Capacity of CAR-T Cells

CAR-T cell are implanted with mitochondria suspended in a solution which comprises 1.2 M proline, 353 mM glucose, 210 mM trehalose, 0.7 mM K-EGTA, 7 mM K-HEPES (pH 7.2) and which has previously undergone a freeze-thaw cycle.

To assess the impact of mitochondria transplantation on CAR-T cell killing capacity, a FACS-based killing assay is performed 24h post transplantation. CAR-T cells (CD19-41BB-CD3z, PMC746) are purchased from ProMab Biotechnologies (Richemond, CA 94806). To measure the percentage of target cells expressing an early maker of apoptosis, in transition to apoptosis or apoptotic, respectively: Annexin V+, Annexin V+/PI+ or PI+ percentages are evaluated. CAR-T cells transplanted or not are plated at an effector to target ratio of 5 to 1. CAR-T cells are co-incubated for 4 h with target cells Daudi (ATCC CCL-213), a B lymphoblast cell line expressing CD19. Post co-incubation, the cells are collected and stained with anti-human CD8 PerCP-Cy5.5 (Stemcell, 60022PS) and anti-human CD3 APC (Stemcell, 60011AZ) for 20 minutes at 4° C. After a wash with FACS buffer, the cells are stained with Annexin V FITC (Biolegend, 640914) and Propidium Iodide (PI, Biolegend, 640914) for 15 minutes at room temperature according to the supplier's instructions and acquired on a FACSLyric (BD Biosciences).

### Procedure

(i) CAR-T cell culture is performed in presence of 100 U/ml of recombinant human IL-2 (Peprotech, 200-02). CAR-T cells are cultured in RPMI 1640 medium GlutaMAX™ Supplement 500 ml (ThermoFisher, 61870010), supplemented with 1% L-glutamine (ThermomFisher, 25030024), 1% penicillin-streptomycin (10,000 U/mL, Gibco, 15140122), 1% non-essential amino acid (NEAA, ThermoFisher, 11140050), 1% sodium pyruvate (ThermoFisher, 11360070), 10% fetal bovine serum and 0.1% 2p-mercaptoethanol (Gibco, 31350-010).
(ii) Mitochondria isolation: Mitochondria are isolated from Human Cardiac Fibroblast (HCF) according to the procedure described in Example 1b.
(iii) Quantification of isolated mitochondria: The mitochondria dosage is estimated in terms of protein content expressed in μg, according to the procedure described in Example 1b.

Mitochondria transplantation enhances the killing capacity of CAR-T cells. Percentages of target cells expressing an early marker of apoptosis, in transition to apoptosis or a late marker of apoptosis are increased upon co-incubation with transplanted CAR-T cells.

### Example 8: Therapeutic Mitochondrial Transplantation (TMT)—Pre-Clinical Study in Pigs

Cryopreserved mitochondria are tested in an animal model of heart ischemia-reperfusion injury. The experiments are performed in an established pig model of regional ischemia-reperfusion injury. Mitochondria are isolated from human cardiac fibroblasts as described in Example 1b. Isolated mitochondria are resuspended in 35 μl of trehalose-based mitochondrial isolation buffer (300 mM trehalose, 1 mM EGTA, 10 mM HEPES, pH 7.2). 10 μl of 3.53 M D-glucose and 5 μl of 6M L-proline are added to the mitochondrial suspension, incubated for 2 minutes, and the tubes are placed on dry ice for freezing. After 10 minutes, the tubes are transferred to storage boxes at −80° C. and kept frozen until use. For animal experiment, heart ischemia is induced in healthy female pigs (Sus scrofa) by occluding coronary artery (LAD) for 90 minutes. Cryopreserved mitochondria (stored at −80° C. not longer than 2 weeks) are rapidly thawed by placing them in a metal block heated to 37° C., immediately resuspended in 5 ml of sucrose-based mitochondrial isolation buffer (300 mM sucrose, 1 mM EGTA, 10 mM HEPES, pH 7.2) and injected into the coronary artery of the pig within 15 minutes after reperfusion Blood samples are taken at Day 0, Day 3, Day 30 and Day 90, and infarct biomarkers (such as creatine kinase-myocardial band (CKMB) or troponin I) are measured in the serum. Heart function is assessed by magnetic resonance imaging (MRI), at Day 3, Day 30 and Day 90 from the start of the treatment by determining functional parameters, such as end systolic volume, end diastolic volume, and ejection fraction. Pigs are sacrificed, and histological samples from the heart and other organs are taken for analysis. Therapeutic effect is assessed by a reduction of serum infarct biomarkers, reduction of infarct size, increase of ejection fraction compared to the control group.

Results: An interim analysis at Day 3 and Day 28, showed the following:
Ejection fraction was measured using magnetic resonance imaging (MRI) in animals treated with the composition stored at −80° C. for up to 2 weeks and thawed before use (n=2) and animals receiving equal volume of the vehicle. MRI was performed on days 3 and 28 after ischemia. Preliminary results indicate that ejection fraction was reduced between days 3 and 28 in two out of three animals receiving vehicle (67%), indicating impairment of heart function, whereas in both animals treated with mitochondria the ejection fraction increased, indicating an improvement.

### Example 9: Spray-Freezing of a Cryopreservative Composition Comprising Isolated Mitochondria

Mitochondria are isolated from HEPG2 cell line stably expressing mitochondria-targeted green fluorescent protein (GFP) by the same method as in Example 1. Isolated mitochondria are resuspended in 350 μl of trehalose-based mitochondrial isolation buffer (300 mM trehalose, 1 mM EGTA, 10 mM HEPES, pH 7.2). Then 100 μl of 3.53 M D-glucose and 50 μl of 6M L-proline are added to the mitochondrial suspension. The composition formed after the addition of glucose and L-proline is then spray-frozen, i.e., the composition is sprayed in the form of droplets directly into liquid nitrogen by using a spraying device. Liquid nitrogen is then allowed to evaporate at room temperature, and the remaining frozen composition is collected into an Eppendorf tube, which has been previously cooled in dry ice. The thawing of the frozen composition is carried out by placing the tube into a metal rack, which has been previously heated to 37° C. Thawed composition was diluted to 1.5 ml with trehalose-based mitochondrial isolation buffer (300 mM trehalose, 1 mM EGTA, 10 mM HEPES, pH 7.2), sedimented by centrifugation for 5 minutes at 9500 g and resuspended in 20 μl of the trehalose-based mitochondrial isolation buffer.

After thawing, the suspension comprising mitochondria is added to a well of a 96-well plate with cultured human cardiac fibroblasts and incubated for 24 hours. Internalization of frozen-thawed GFP-labelled mitochondria is observed using a fluorescence microscope after four washes of the cultured cells with warm cell culture medium. The experiment demonstrates that mitochondria comprised in the composition (i.e., cryopreservative composition containing trehalose, glucose and proline) spray-frozen into liquid nitrogen maintain capability to be internalized by living cells.

### OTHER EMBODIMENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and sub-combinations of the various elements, features, functions, and/or properties disclosed herein. The following claims particularly point out certain combinations and sub-combinations regarded as novel and nonobvious. Inventions embodied in other combinations and sub-combinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed is:

1.-94. (canceled)

95. A composition comprising isolated human mitochondria and
(i) an aqueous buffer having a pH of 5.5 to 8.5;
(ii) trehalose at a concentration of at least 150 mM; and
(iii) a first cryoprotecting agent(s) selected from one or more (a) amino acid(s) at a total concentration of at least 160 mM.

96. The composition according to claim 95, further comprising
(iv) a second cryoprotecting agent(s) selected from one or more (b) sugar(s), (c) polymer(s), or a combination thereof, wherein
(b) the sugar(s) has a total concentration of at least 150 mM; and
(c) the polymer(s) has a total concentration of 2.5% (w/v) to 30% (w/v).

97. The composition of any one of claim 95 or 96,
wherein (i) the aqueous buffer comprises a buffering agent, wherein the buffering agent is selected from 2-[4-(2-hydroxyethyl)-piperazin-1-yl]-ethane-sulfonic acid (HEPES), piperazine-N,N′-bis(2-ethane-sulfonic acid) (PIPES), 4-morpholineethanesulfonic acid (MES), bis-(2-hydroxyethyl)amino-tris-(hydroxymethyl)-methane (Bis-Tris), 2-(N-cyclohexylamino)-ethane sulfonic acid (CHES), N,N-Bis-(2-hydroxyethyl)-glycine (Bicine), potassium phosphate, sodium cacodylate, tris-(hydroxymethyl)aminomethane hydrochloride) (Tris), 4-morpholinepropanesulfonic acid (MOPS), 1,3-bis-[tris-(hydroxymethyl)-methylamino]-propane (Bis-Tris propane), sodium acetate, or a combination thereof, preferably HEPES; and
wherein the buffering agent in the aqueous buffer has a concentration of 0.5 mM to 50 mM.

98. The composition of any one of claims 95 to 97, further comprising a calcium chelator, wherein the calcium chelator
is selected from the group consisting of ethylene glycol-bis(3-aminoethyl ether)-N,N,N′,N′-tetraacetic acid (EGTA), 2,2′,2″,2′″-(Ethane-1,2-diyldinitrilo)-tetraacetic acid (EDTA), 1,2-bis(o-aminophenoxy)ethane-N,N,N′,N′-tetraacetic acid (BAPTA), 1,2-bis(2-aminophenoxy)ethane-N,N,N′,N′-tetraacetic acid tetrakis-(acetoxymethyl ester) (BAPTA-AM) or a combination thereof, preferably is selected from EGTA or EDTA; and/or
has a concentration of at a concentration of 0.1 mM to 10 mM.

99. The composition of any one of claims 95 to 98, further comprising a ionic component, wherein the ionic component is:
at a concentration of 0.1 mM to 100 mM; and/or
selected from salts, acids, or bases comprising Mg²⁺, Na⁺, K⁺, Cl⁻, HCO₃⁻, such as MgCl₂. MgSO₄, KCl, KH₂PO₄, NaHCO₃, Na₂HPO₄, formate anions, e.g., C₂H₂MgO₄ (magnesium formate), pyruvate anions, e.g., C₃H₃NaO₃ (sodium pyruvate), acetate anions, e.g., C₂H₃NaO₂ (sodium acetate), malate anions, oxaloacetate anions, glutamate anions, α-ketoglutarate anions, succinate anions, or a combination thereof.

100. The composition of any one of claims 95 to 99, further comprising albumin at a concentration of 0.01% (w/v) to 10% (w/v), wherein albumin is preferably bovine serum albumin (BSA), human serum albumin (HSA), or a combination thereof.

101. The composition of any one of claim 95 or 96, wherein (i) the buffer has
pH 7.4 and comprises 20 mM Tris, 2 mM EDTA, and 10 mM MgCl₂;
pH 7.25 and comprises 5 mM MOPS, 10 mM BAPTA, and 5 mM sodium pyruvate; or
pH 7.2 and comprises 10 mM HEPES, and 1 mM EGTA.

102. The composition of any one of claims 95 to 101, wherein the composition includes less than a cryopreservative amount of propylene glycol, ethylene glycol, glycerol and dimethyl sulfoxide (DMSO), or no propylene glycol, ethylene glycol, glycerol and dimethyl sulfoxide (DMSO).

103. The composition of any one of claims 95 to 102, wherein
(a) the amino acid(s) is selected from leucine, isoleucine (e.g., L-isoleucine), proline (e.g., L-proline), methylproline, benzylproline, hydroxyproline, aminoproline, dehydroproline, aziridinecarboxylic acid, azetidinecarboxylic acid, pipecolic acid, oxaproline, thiaproline, valine (e.g., L-valine), alanine (e.g., L-alanine), glycine, asparagine (e.g., L-asparagine), aspartic acid (e.g., L-aspartic acid), glutamic acid (e.g., L-glutamic acid), serine (e.g., L-serine), histidine (e.g., L-histidine), cysteine (e.g., L-cysteine), tryptophan (e.g., L-tryptophan), tyrosine (e.g., L-tyrosine), arginine (e.g., L-arginine), glutamine (e.g., L-glutamine), lysin, threonine, selenocysteine, methionine, phenylalanine, creatine (e.g., L-creatine), taurine (e.g., L-taurine), betaine, ectoine, dimethylglycine, ethylmethylglycine, an RGD peptide, or a combination thereof.

104. The composition of claim 103, wherein
(a) the amino acid(s) is selected from methylproline, benzylproline, hydroxyproline, aminoproline, dehydroproline, aziridinecarboxylic acid, azetidinecarboxylic acid, pipecolic acid, oxaproline, thiaproline, or a combination thereof.

105. The composition of claim 103, wherein (a) the amino acid is proline (e.g., L-proline).

106. The composition of any one of claims 95 to 105, wherein (a) the amino acid(s) has a concentration of at least 180 mM or at least 200 mM.

107. The composition of any one of claims 95 to 105, wherein (a) the amino acid(s) has a concentration of at least 1000 mM.

108. The composition of any one of claims 96 to 107, wherein (b) the sugar(s) is selected from: mono-, di-, or trisaccharide, or a combination thereof.

109. The composition of any one of claims 96 to 108, wherein (b) the sugar(s) is selected from maltose, lactose, fructose, sucrose, glucose, dextran, melezitose, raffinose, nigerotriose, maltotriose, maltotriulose, kestose, cellobiose, chitobiose, lactulose, or a combination thereof.

110. The composition of any one of claims 96 to 109, wherein (b) the sugar(s) is selected from sucrose, glucose, or a combination thereof.

111. The composition of any one of claims 96 to 110, wherein (c) the polymer is
a biocompatible, hydrophilic, amphiphilic polymer, or a combination thereof, and
is selected from: poloxamer, such poloxamer 142, poloxamer 188, poloxamer 331, or poloxamer 407, alginate, polyethylene glycol (PEG), such as PEG400 or PEG1000, polyglutamic acid, polyvinyl alcohol, polyvinyl pyrrolidone, or a combination thereof, preferably wherein (c) the polymer is polyethylene glycol (PEG).

112. The composition of any one of claims 96 to 111, wherein
(iii) the first cryoprotecting agent is (a) one or more amino acid(s), wherein the amino acid is
wherein the amino acid is selected from proline, methylproline, benzylproline, hydroxyproline, aminoproline, dehydroproline, aziridinecarboxylic acid, azetidinecarboxylic acid, pipecolic acid, oxaproline, thiaproline, or a combination thereof;
and wherein
(iv) the second cryoprotecting agent(s) is selected from one or more (b) sugar(s), (c) polymer(s), or a combination thereof, wherein
(b) the sugar(s) is glucose, sucrose, or a combination thereof; and
(c) the polymer is polyethylene glycol (PEG).

113. The composition according to claim 112, wherein
(a) the amino acid is proline at a concentration of at least 500 mM or at least 1000 mM.

114. The composition according to claim 112, wherein
the amino acid is proline at a concentration of at least 1300 mM or at least 1600 mM.

115. The composition of any one of claims 112 to 114 wherein
the amino acid is proline; and
the sugar is glucose at a concentration of 200 mM to 1300 mM or is sucrose at a concentration of 160 mM to 900 mM.

116. The composition of any one of claims 95 to 114, wherein
the amino acid is proline; and
the polymer is polyethylene glycol (PEG) at a concentration of 5% (w/v) to 30% (w/v).

117. The composition of any one of claims 95 to 102, 107, 108, or 112, wherein the cryoprotecting agent consists of proline at a concentration according to any one of claim 113 or 114.

118. The composition of claim 116, wherein proline is at a concentration of 600 mM or 1200 mM.

119. The composition of any one of claims 95 to 102, 104, 106, or 112, wherein the cryoprotecting agent consists of one or more (a) amino acid(s) selected from methylproline, benzylproline, hydroxyproline, aminoproline, dehydroproline, aziridinecarboxylic acid, azetidinecarboxylic acid, pipecolic acid, oxaproline, thiaproline, or a combination thereof, at a total concentration of at least 200 mM.

120. The composition of any one of claims 95 to 102, 112, or 119, wherein the cryoprotecting agent(s) consists of one or more (a) an amino acid(s), wherein
the amino acid is selected from methylproline, benzylproline, hydroxyproline, aminoproline, dehydroproline, aziridinecarboxylic acid, azetidinecarboxylic acid, pipecolic acid, oxaproline, thiaproline, or a combination thereof, at a concentration of at least 300 mM or of at least 500 mM.

121. The composition of any one of claims 95 to 102, 112, or 119, wherein the cryoprotecting agent consists of one or more (a) amino acid(s), wherein
the amino acid is selected from: methylproline, benzylproline, hydroxyproline, aminoproline, dehydroproline, aziridinecarboxylic acid, azetidinecarboxylic acid, pipecolic acid, oxaproline, thiaproline, or a combination thereof, at a concentration of at least 1000 mM or of at least 1500 mM.

122. The composition of any one of claims 95 to 121, wherein the mitochondria have been isolated from cells, tissues, or organs.

123. The composition of any one of claims 95 to 122, wherein the isolated mitochondria, have a concentration of at least 0.02 μg/μL.

124. The composition of anyone of claims 95 to 123, wherein the isolated mitochondria have a concentration of no more than 100 μg/μL.

125. The composition of any one of claims 95 to 124, wherein the isolated mitochondria are linked to
(i) a pharmaceutical agent, diagnostic agent, imaging agent, therapeutic agent, or any other biocompatible agent;
(ii) an antibody; or
(iii) an antigen binding fragment, wherein the antigen binding fragment is at least one portion of an antibody or TCR, or recombinant variants thereof.

126. The composition of claim 125, wherein the mitochondria are linked to the agent, the antibody, or the antigen binding fragment, by a covalent bond or by a non-covalent bond (e.g., electrostatic bond).

127. The composition of claim 125, wherein the agent, the antibody, or the antigen, are
(i) embedded in the mitochondria, embedded in the mitochondrial membrane, substantially enclosed within a mitochondrion, or encapsulated entirely by mitochondria; or
(ii) linked to the outer membrane of mitochondria by a covalent or a non-covalent bond, such as an electrostatic bond.

128. The composition of any one of claims 95 to 127, wherein the isolated mitochondria
(i) are mitochondria modified by gene editing; or
(ii) comprise exogenous mtDNA.

129. A method for the cryopreservation of a composition comprising isolated mitochondria, according to any one of claims 95 to 128, the method comprising the steps of:
(a) freezing the composition at a temperature below 0° C.; and
(b) storing the frozen composition obtained according to step (a) at a temperature below 0° C.

130. The method for the cryopreservation of a composition according to claim 129, further comprising the step of: (c) thawing the frozen composition at a temperature above 0° C.

131. The method of claim 129, wherein
(a) freezing is at a temperature below −20° C. or below −100° C.

132. The method of any one of claims 129 to 131 wherein
(a) freezing is done in liquid nitrogen at a temperature of −196° C. or in dry ice at a temperature of −78.5° C., preferably in dry ice at −78.5° C.

133. The method of any one of claims 129 to 132, wherein
(a) freezing is snap-freezing; or
(a) freezing is a gradual freezing at a rate of at least 5° C./min.

134. The method of any one of claims 129 to 133, wherein
(b) storing has a duration of a period of at least 24 hours or of at least 1 week.

135. The method of any one of claims 129 to 133, wherein
(c) thawing is performed at a temperature higher than 4° C. and lower than 40° C.

136. A composition according to any one of claims 95 to 128 or a composition prepared by the method of any one of claims 129 to 135, in a therapeutically effective amount for use in the treatment of a disease.

137. The composition according to claim 136 for use in the treatment of
(i) a mitochondrial or mitochondrial-related disease;
(ii) cancer or tumor;
(iii) autoimmune disease;
(iv) ischemia related injuries, such as lung-, kidney-, cardiac-, or brain-ischemia-reperfusion injuries; or
(v) blockages in the blood vessels;
in a subject in need thereof.

138. The composition according to claim 136 for use in gene therapies.

139. The composition according to claim 138 for use in gene therapy for the treatment of cancer, infectious diseases, or autoimmune diseases.

140. The composition according to claim 136 for use in the treatment of a disease in a subject in need, wherein said composition is to be administered to a subject in need
(i) by topical or parental administration;
(ii) by direct injection into a blood vessel, a tissue, or an organ; or
(iii) in the form of an aerosol.

141. The composition according to any one of claims 95 to 128 and claim 136, wherein the mitochondria, comprised in the composition are autologous, allogeneic, or xenogeneic.

142. The composition according to any one of claims 136 to 141, wherein the composition has undergone at least a cycle of freeze-thaw (e.g., a freeze-thaw cycle according to the method of any one of claims 131 to 135), prior to use in the treatment of the disease.

143. Use of the composition of any one of claims 95 to 128 or of the composition prepared by the method of any one of claims 129 to 135 for the cryopreservation of viable mitochondria.
